(19) United States
(12) Patent Application Publication
(10) Pub. No.: US 2026/0140101 A1
(43) Pub. Date: May 21, 2026

(54) Integrated Sensing System for Water Quality Monitoring and Management

(71) Applicants: BOARD OF REGENTS, THE UNIVERSITY OF TEXAS SYSTEM, Austin, TX (US); THE UNITED STATES OF AMERICA, AS REPRESENTED BY THE SECRETARY OF AGRICULTURE, Washington, DC (US)

(72) Inventors: Shawana Tabassum, Tyler, TX (US); Brian David Ott, Stoneville, MS (US); A K M Sarwar Inam, Tyler, TX (US); Prasanna Gowda, Arlington, TX (US)

(73) Assignees: BOARD OF REGENTS, THE UNIVERSITY OF TEXAS SYSTEM, Austin, TX (US); THE UNITED STATES OF AMERICA, AS REPRESENTED BY THE SECRETARY OF AGRICULTURE, Washington, DC (US)

(21) Appl. No.: 19/328,325

(22) Filed: Sep. 15, 2025

Related U.S. Application Data
(60) Provisional application No. 63/720,978, filed on Nov. 15, 2024.

(51) Int. Cl. G01N 33/18; G01N 27/416

(52) U.S. Cl. CPC G01N 33/1886; G01N 27/4167; G01N 33/1813

(57) ABSTRACT

A multi-parametric water sensor including a substrate; a sensor array; and an enclosure comprising a water impermeable material. The sensor array comprises an analyte sensor comprising a working electrode (WE) and a reference electrode (RE) disposed on a first surface of the substrate; a pH sensor comprising a working electrode (WE) and a reference electrode (RE) disposed on the first surface or a second surface of the substrate; and a temperature sensor disposed on the first surface or a second surface of the substrate. The enclosure may include a sensor section and a water permeable section. Methods of measuring one or more analytes in an aqueous environment and determining a presence or an amount of ammonia (NH₃) in an aqueous environment.

### CROSS-REFERENCE TO RELATED APPLICATION

This application claims priority benefit to U.S. Provisional application No. 63/720,978, filed Nov. 15, 2024, the disclosure of which is incorporated herein by reference in its entirety.

### STATEMENT REGARDING FEDERALLY SPONSORED RESEARCH OR DEVELOPMENT

This invention was made with Federal government support under USDA-ARS 58-6066-2-042 awarded by the United States Department of Agriculture. The government has certain rights in the invention.

### COLOR DRAWINGS

The patent or application file contains at least one drawing executed in color. Copies of this patent or patent application publication with color drawing(s) will be provided by the Office upon request and payment of the necessary fee.

### TECHNICAL FIELD

A sensor suite capable of simultaneously measuring multiple conditions in various water-based systems.

### BACKGROUND

Ammonia comes from multiple sources including fish excretion, sediment, and decomposition of organic matter. Ammonia is usually measured as “total ammonia,” which is the sum of two forms in water:ammonium (NH₄⁺) and ammonia (NH₃). Ammonium is an ion and is largely non-toxic to fishes, whereas ammonia, also known as un-ionized or toxic ammonia, is a gas that easily crosses fish gills and is extremely toxic. The ratio of ammonium and ammonia is in equilibrium with pH and temperature; when either or both pH or temperature increase, the proportion of ammonia increases. Low levels of ammonia can become toxic, leading to stress, reduced growth, and increased disease susceptibility. Nitrate (NO₃⁻) is a less toxic ion produced by nitrification, but can still pose health risks at high levels, and along with ammonium can cause algal blooms that can deplete oxygen and create hypoxic conditions harmful to fish and aquatic life. Nitrification is an important component of the nitrogen cycle and is an important process for aquaculture, agriculture, ecology, and wastewater management. Therefore, it is crucial to measure nitrate and total ammonia in aquaculture.

Technology to accurately monitor nitrate and total ammonia levels is needed to ensure that the water remains within a safe range for fish and other aquatic organisms. Elevated levels of these solutes causes stress, suppress immune function, and hinder growth. Chronic exposure to suboptimal water quality can lead to poor feed conversion efficiency, reduced reproductive success, and higher mortality rates.

In recirculating aquaculture systems (RAS), where water reuse is a key component, maintaining low levels of total ammonia and nitrate is essential to ensure system efficiency and sustainability. The ability to effectively manage these compounds reduces the need for frequent water changes, conserves water resources, and lowers operational costs. There is an unmet need for technology to monitor and control these conditions so that aquaculture operations can balance between fish density, feed input, and waste management, to achieve more sustainable practices.

Additionally, managing nitrate and total ammonia levels is important for minimizing the environmental impact of aquaculture. Discharge of nutrient-rich water into natural water bodies can contribute to eutrophication, leading to algal blooms, oxygen depletion, and the disruption of local ecosystems. There is an unmet need to accurately monitor and manage these levels in aquaculture facilities.

### SUMMARY

In one aspect, the disclosure provides a multi-parametric water sensor, comprising a substrate; a sensor array; and an enclosure comprising a water impermeable material. In some aspects, sensor array comprises an analyte sensor comprising a working electrode (WE) and a reference electrode (RE) disposed on a first surface of the substrate; a pH sensor comprising a working electrode (WE) and a reference electrode (RE) disposed on the first surface or a second surface of the substrate; and a temperature sensor disposed on the first surface or a second surface of the substrate. In some aspects, the enclosure comprises a sensor section and a water permeable section.

In one aspect, the disclosure provides a multi-parametric water sensor, comprising a substrate; a sensor array; and an enclosure comprising a water impermeable material. In some aspects, the sensor array comprises a first analyte sensor comprising a working electrode (WE), a counter electrode (CE), and a reference electrode (RE) disposed on a first surface of the substrate; a second analyte sensor comprising a working electrode (WE), a counter electrode (CE), and a reference electrode (RE) disposed on the first surface of the substrate; a pH sensor comprising a working electrode (WE) and a reference electrode (RE) disposed on a second surface of the substrate; and a temperature sensor disposed on the second surface of the substrate. In some aspects, the enclosure comprises a sensor section and a water permeable section.

In one aspect, the disclosure provides a method of measuring one or more analytes in an aqueous environment, comprising placing the multi-parametric water sensor according to any one or combination of aspects disclosed herein in the aqueous environment; measuring a signal corresponding to one or more analytes in the aqueous environment; measuring at least one environmental parameter comprising:
i) measuring a signal corresponding to a pH of the aqueous environment;
ii) measuring a signal corresponding to a temperature of the aqueous environment; or
iii) both i) and ii); and
   obtaining as a data output from the multi-parametric water sensor an amount of the one or more analytes in the aqueous environment, wherein the amount of at least one of the analytes is based on the measured value of the at least one environmental parameter of the aqueous environment. In one aspect, the disclosure provides a

In one aspect, the disclosure provides method of determining a presence or an amount of ammonia (NH₃) in an aqueous environment based on the amount of total ammonia using the multi-parametric water sensor according to any one or combination of aspects disclosed herein.

### BRIEF DESCRIPTION OF THE DRAWINGS

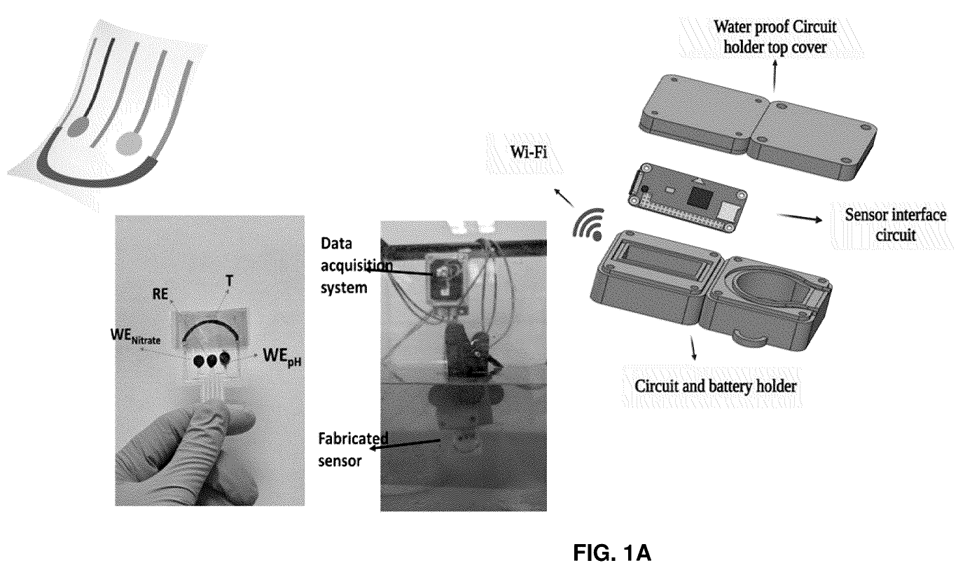

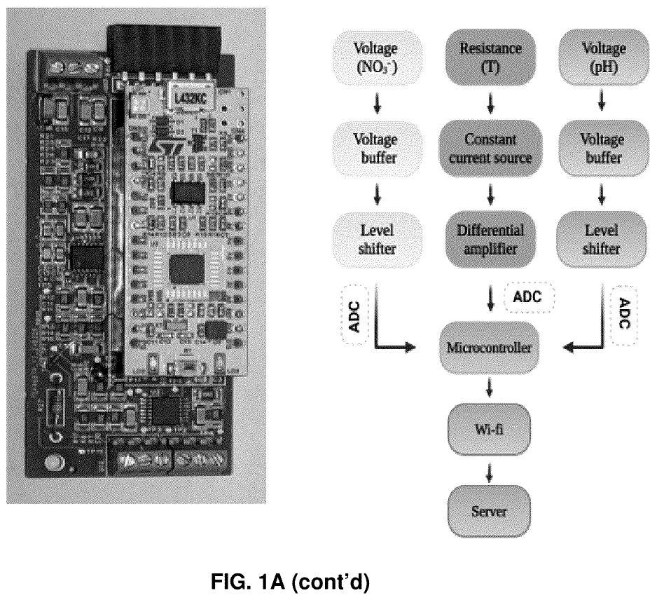

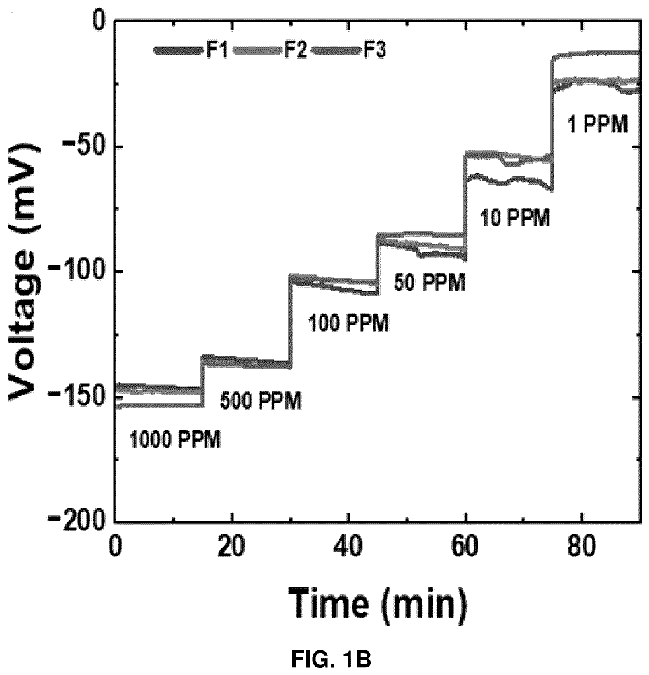

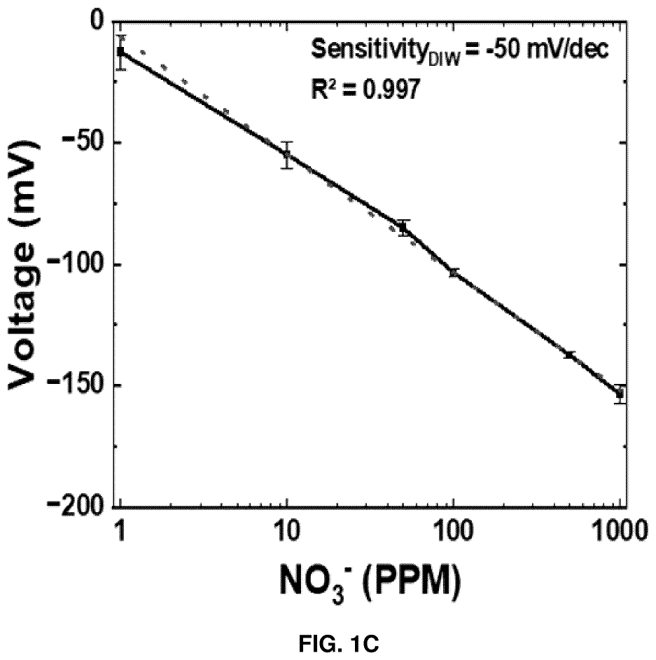

FIGS. 1A-1C show a schematic of a flexible sensor array (FIG. 1A), dynamic responses of three identical sensors to nitrate concentrations ranging from 1 to 1000 ppm with measurements recorded for 15 minutes at each concentration (FIG. 1B), and the average voltage readings from the three sensors were plotted against the logarithm of NO₃⁻ concentrations in ppm, with error bars calculated from three sensor measurements for each concentration (FIG. 1 C).

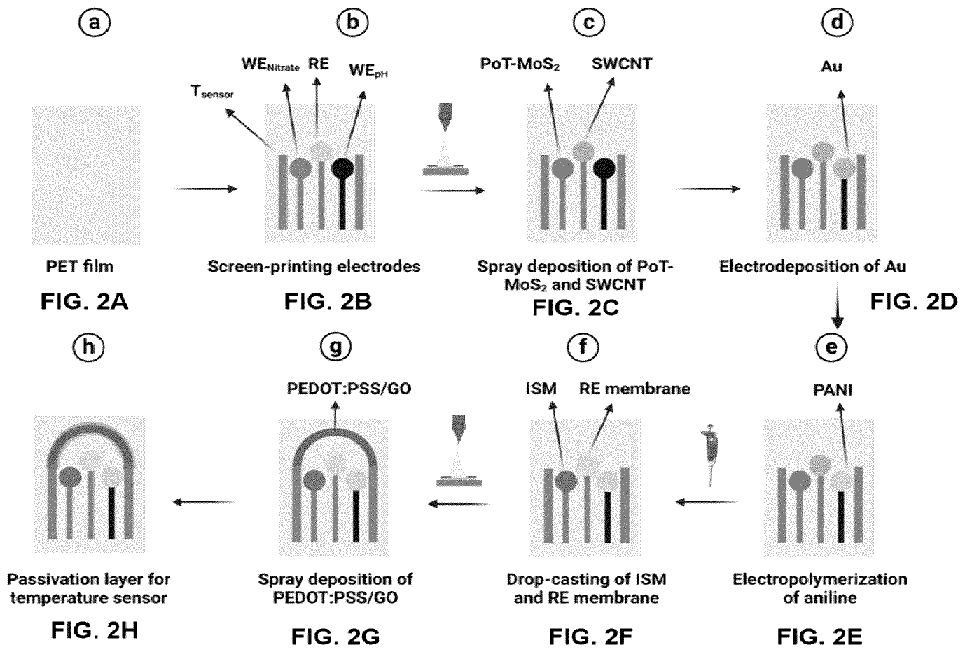

FIGS. 2A-2H shows a fabrication process of a multiparametric sensor. PET film (FIG. 2A). Screen-printing of electrodes (FIG. 2B). Spray deposition of PoT-MoS2 upon WENitrate and SWCNT upon RE (FIG. 2C). Electrodeposition of Au on top of carbon WEpH (FIG. 2D). Electropolymerizing of aniline into polyaniline on WEpH (FIG. 2E). Drop casting of ISM on WENitrate and RE membrane on RE (FIG. 2F). Spray deposition of temperature sensitive PEDOT:PSS/GO (FIG. 2G). Passivation layer on top of temperature selective coating (FIG. 2H).

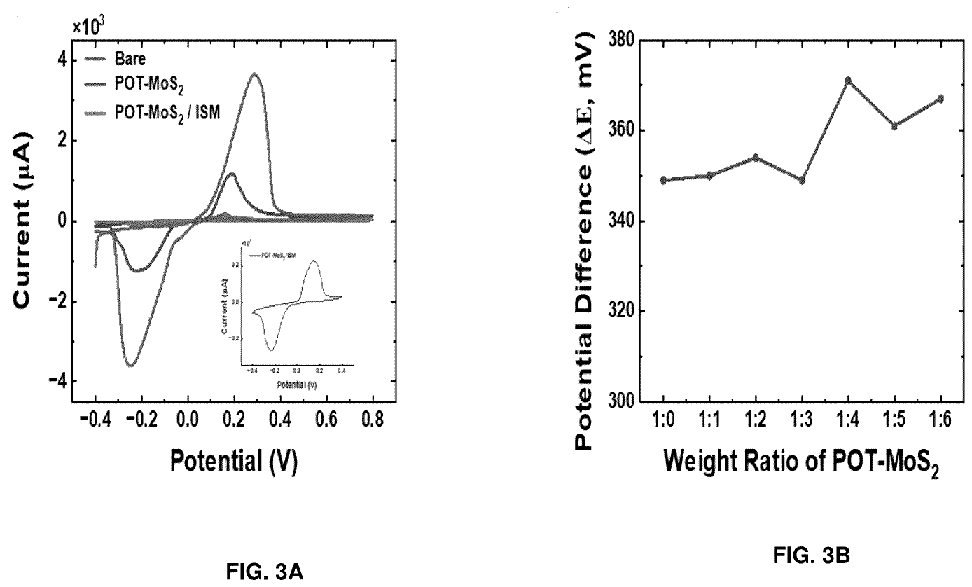

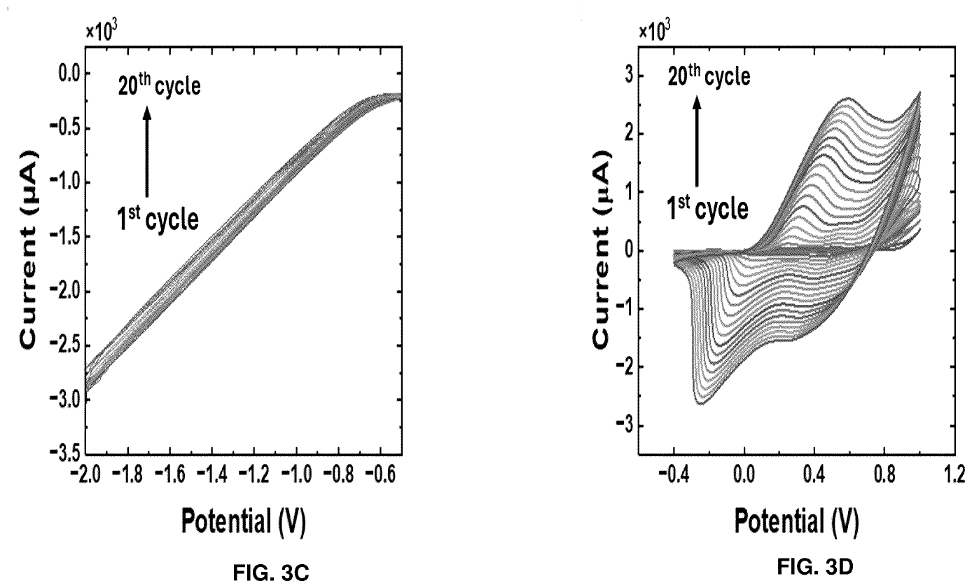

FIGS. 3A-3D shows cyclic voltammograms (CV) were recorded for a range of electrodes, including Bare Ag electrodes, PoT-MoS₂, and PoT-MOS₂/ISM (FIG. 3A) with CV curves specifically for the PoT-MoS₂/ISM configuration shown in the inset. The potential differences (ΔE) derived from the CV curves plotted against the ratio of PoT-MoS₂-based electrodes varied from 1:0 to 1:6 by weight percentages (FIG. 3B). CV plots for electrodeposited Au (FIG. 3C). The process of polymerization of aniline on the Au layer's surface with an increase in current with each cycle indicating the deposition of polyaniline (FIG. 3D).

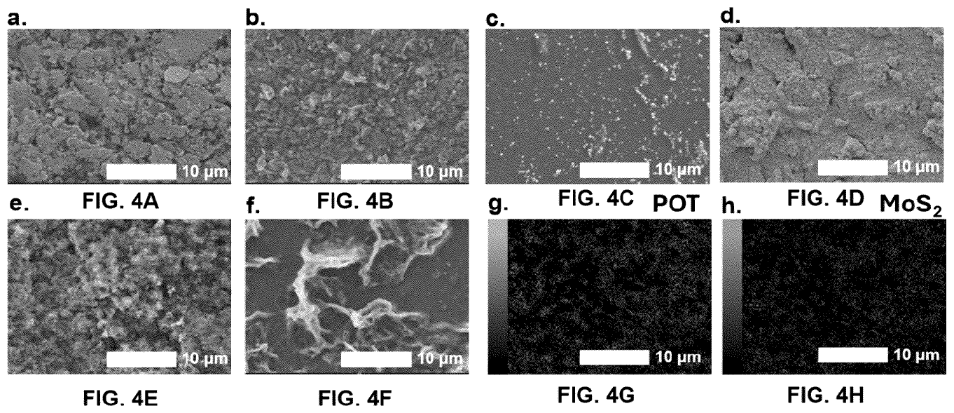

FIGS. 4A-4I shows a higher magnification scanning electron microscopy (SEM) images for Ag film (FIG. 4A), which was crystalline in nature. MoS₂ nanosheet embedded in POT matrix (FIG. 4B). Nanoporous ISM layer (FIG. 4C). Surface microstructure on Au-coated carbon electrode (FIG. 4D). Au electrode after the deposition of electropolymerized aniline (FIG. 4E). GO/PEDOT:PSS (FIG. 4F). Elemental map analysis of POT film (FIG. 4G) and MoS₂ film (FIG. 4H). EDS results showing the mass percentage of Sulfur and Molybdenum (FIG. 4I).

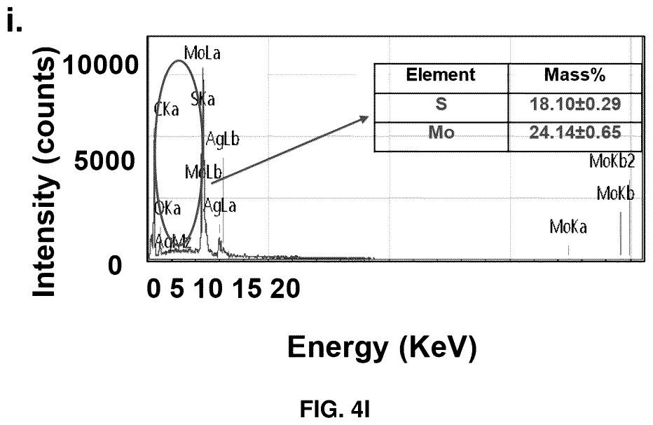

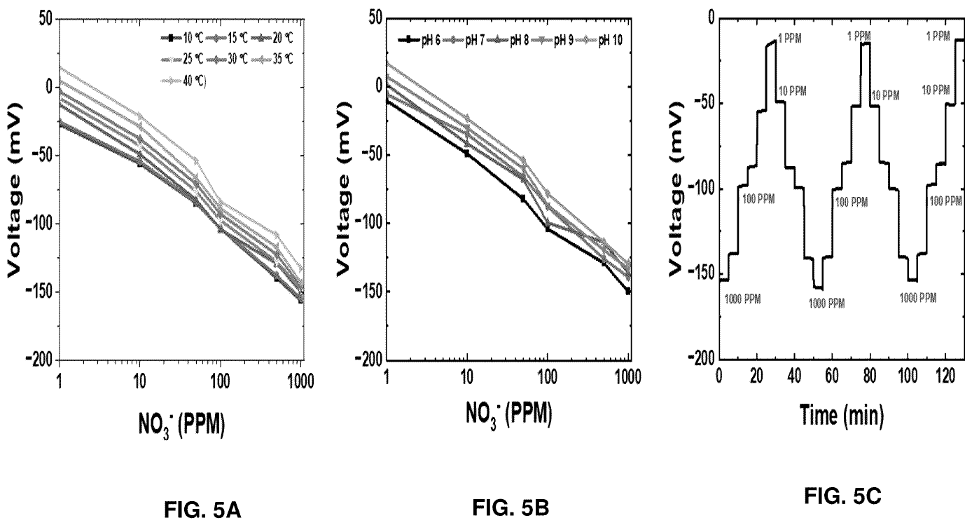

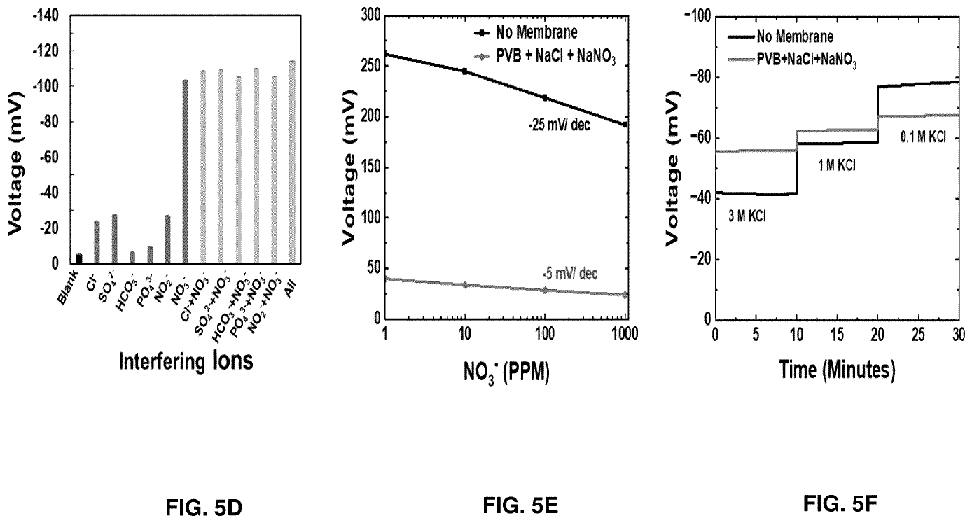

FIGS. 5A-5F show calibration curves of the nitrate sensor under varying solution temperatures (FIG. 5A), calibration curves of the nitrate sensor under varying solution pH (FIG. 5B), cyclic response of the sensor in response to increasing and decreasing NO₃⁻ concentrations (FIG. 5C), the selectivity study where the concentration of NO₃⁻ and the interfering ions was set to 100 ppm, the sensor was first exposed to individual interfering ions, and subsequently, it was tested in a mixture containing both the interfering ions and NO₃⁻ (FIG. 5D), the Ag/AgCl RE electrode with a PVB membrane coating demonstrated minimal sensitivity to NO3-concentration changes, exhibiting a slope of −5 mV/dec; while the bare Ag/AgCl RE electrode displayed a steeper slope of −25 mV/dec (FIG. 5E), and the RE electrode without the PVB membrane exhibited considerable variability across different KCl concentrations, highlighting the instability of the bare Ag/AgCl electrode (FIG. 5F).

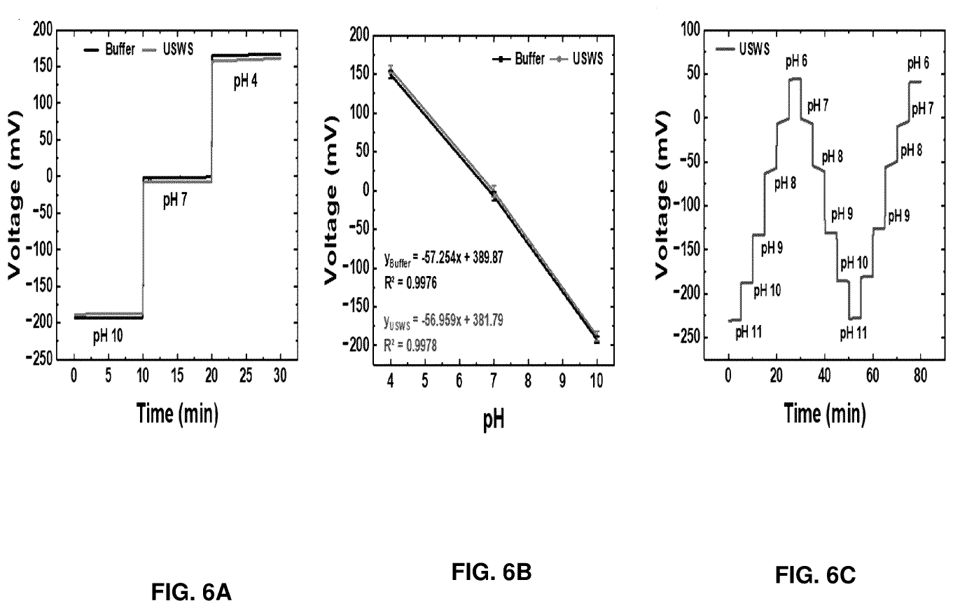

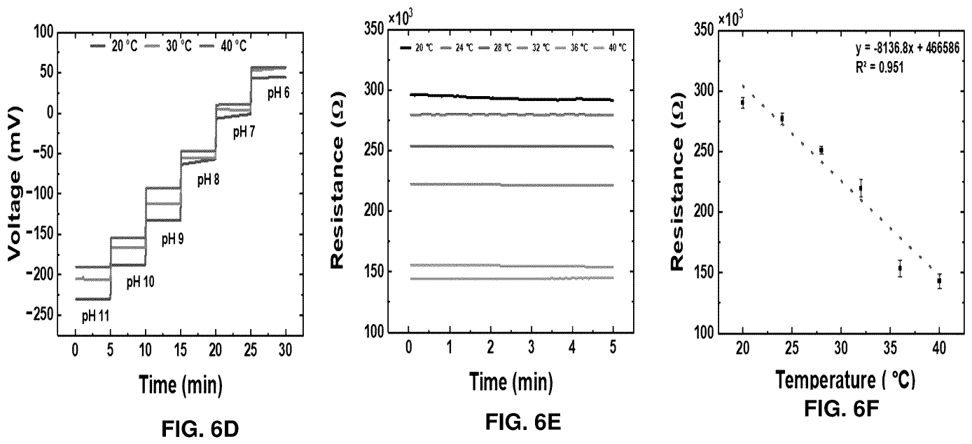

FIGS. 6A-6F show the dynamic response of the pH sensor in both buffer solutions and USDA water samples in response to pH 4, pH 7, and pH 10 (FIG. 6A), calibration plot of the pH sensor, with error bars derived from three consecutive measurements at each pH level (FIG. 6B), cyclic response of the pH sensor to varying pH levels in USDA water samples (FIG. 6C), dynamic response of the pH sensor to variations in solution temperature (FIG. 6D), dynamic response of the temperature sensor over a temperature range of 20° C. to 40° C., with the sensor immersed in the USDA water sample (FIG. 6E), and calibration plot of the GO/PEDOT:PSS-based temperature sensor (FIG. 6F).

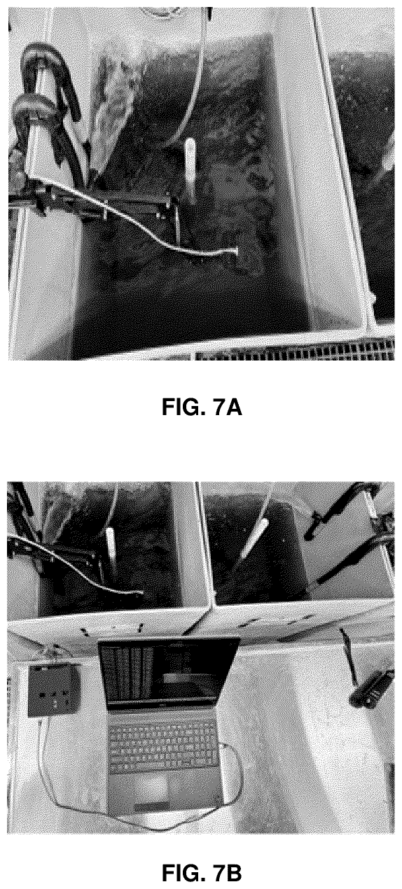

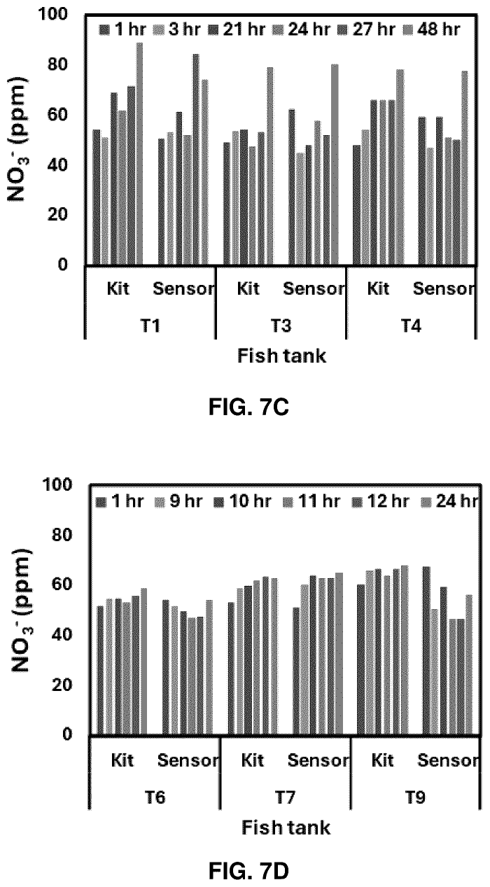

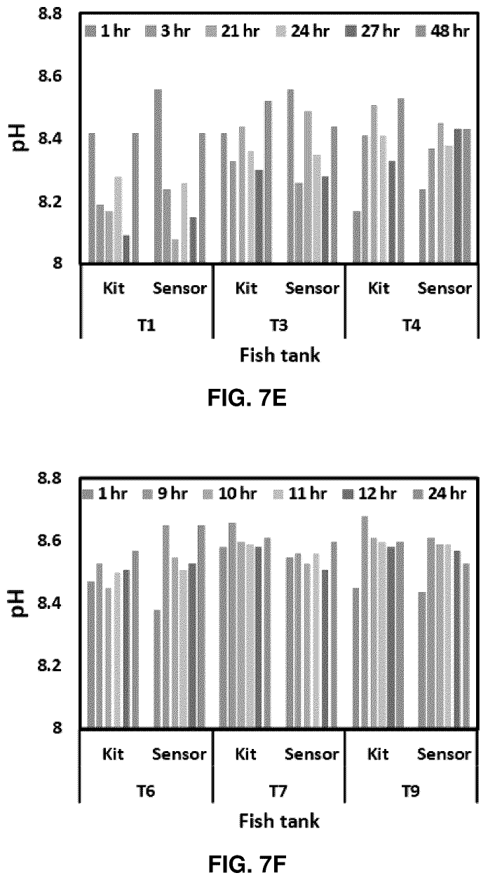

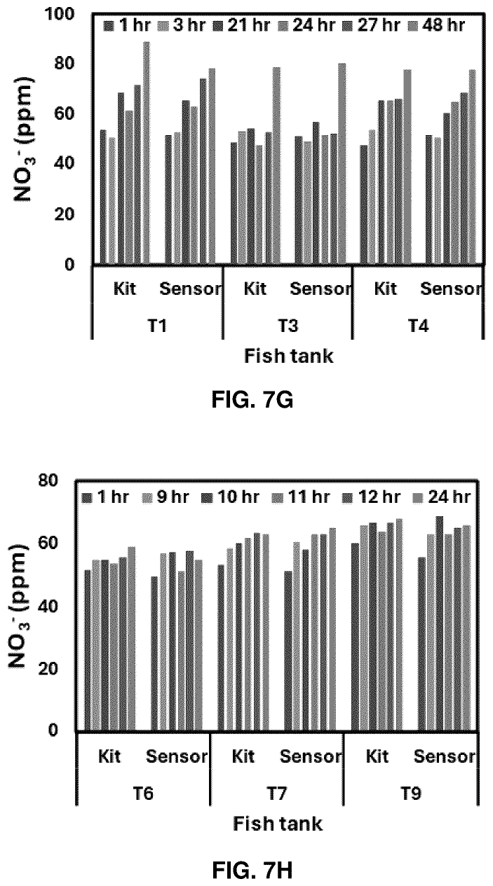

FIG. 7A-7H show a 3D-printed water-resistant box enclosure housing the sensor array deployed in USDA catfish RAS (FIG. 7A), the complete system collects data from the sensor array and transmits data to the PC (FIG. 7B), bar plots comparing the fabricated nitrate sensor with the commercial nitrate measurement kit, showing hourly variations in nitrate concentrations over 48 hours for tanks T1, T3, and T4 (FIG. 7C), similar tests for tanks T6, T7, and T9 were conducted, but for 24 hours (FIG. 7D), the fabricated pH sensor was evaluated against a commercial pH meter over 48 hours for tanks T1, T3, and T4 (FIG. 7E), similar tests for tanks T6, T7, and T9 were conducted for 24 hours (FIG. 7F), pH corrected bar plots comparing the nitrate sensor and commercial kit measurements for tests conducted in tanks T1, T3, and T4 (FIG. 7G), and tanks T6, T7, and T9 (FIG. 7H).

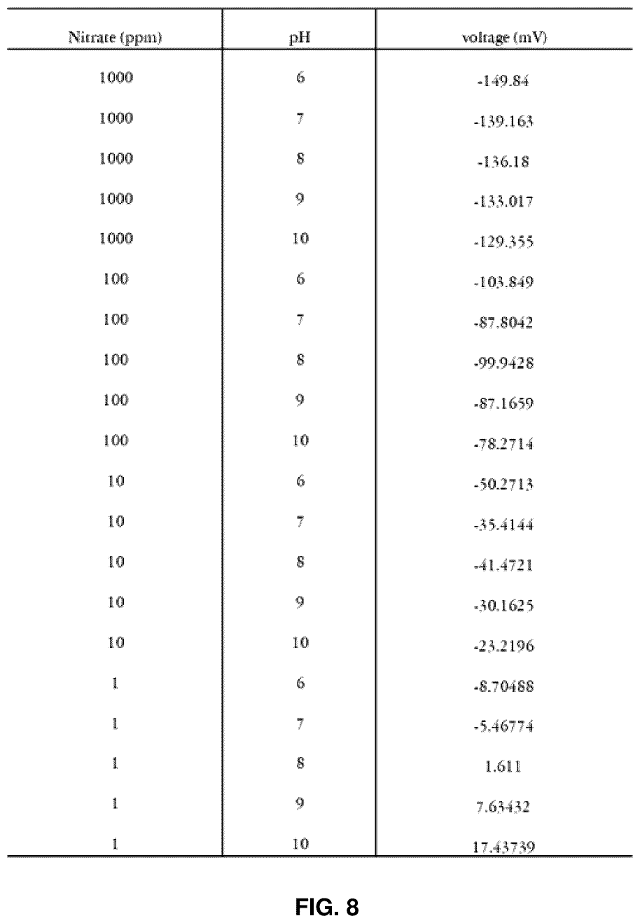

FIG. 8 shows data points from the calibration plot for nitrate sensor in the range of 1000 ppm-1 ppm and corresponding water sample's pH ranging from pH 6 to pH 10.

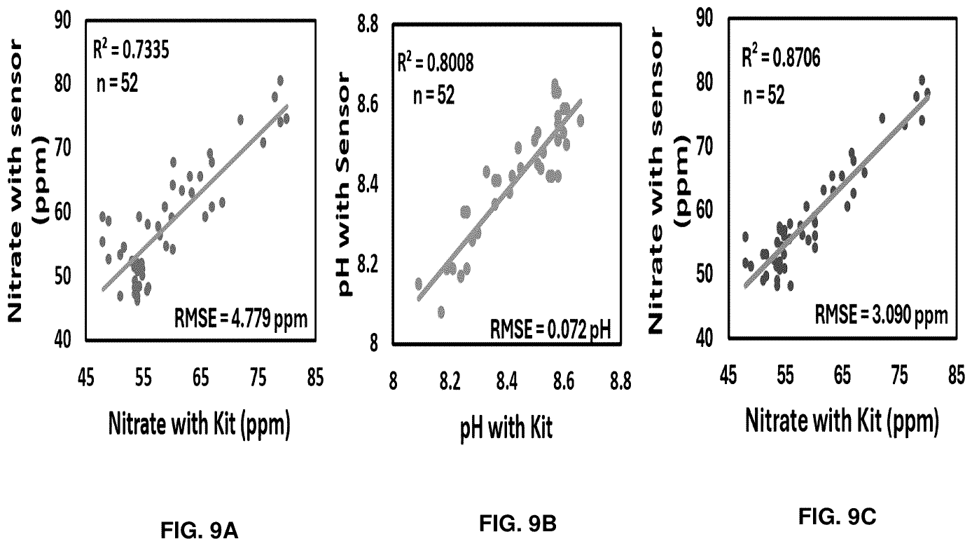

FIGS. 9A-9C show scatter plots of nitrate and pH values measured with the sensors against the values obtained from commercial kits. The nitrate sensor showed a relatively low R2 value and high RMSE due to the pH fluctuations in the tanks (FIG. 9A), the pH sensor attained a high R2 value of 0.8008 and a low RMSE value of 0.072 (FIG. 9B), following pH corrections, the nitrate sensor shows a strong correlation with the kit values, and the R2 value significantly increased, while RMSE decreased post-correction (FIG. 9C).

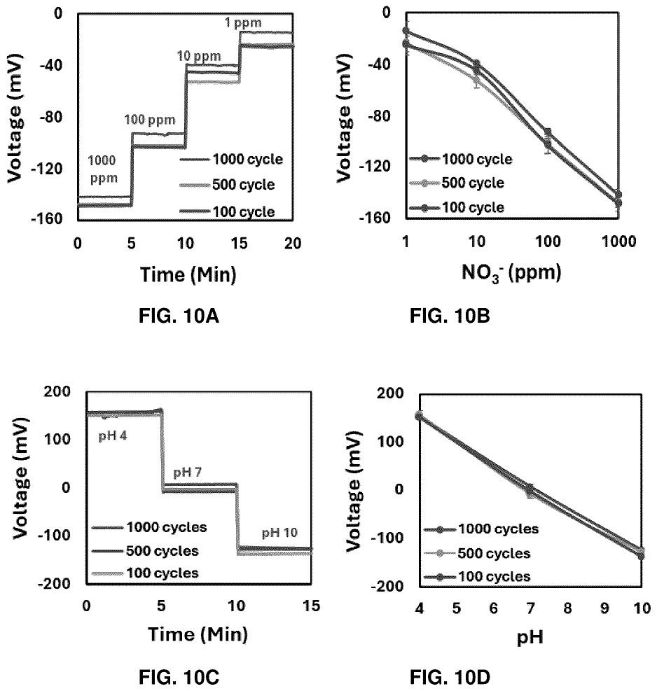

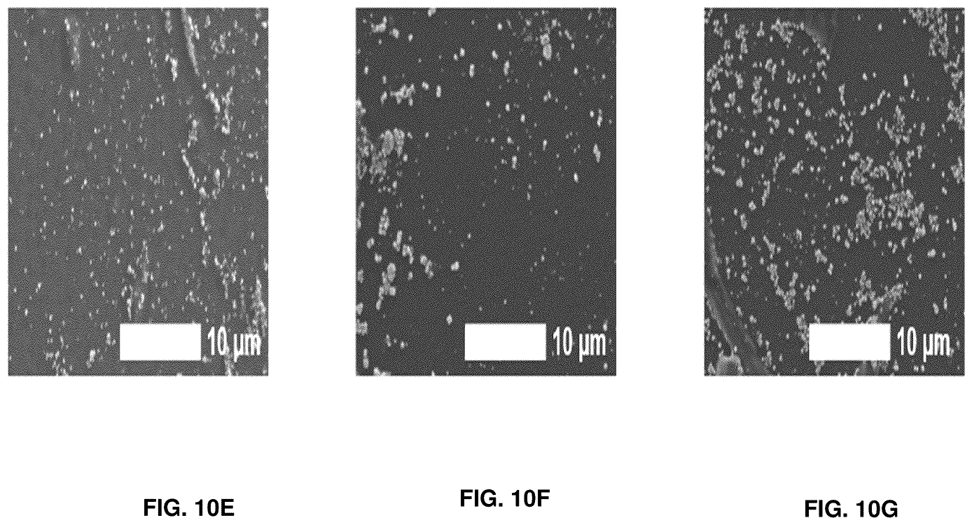

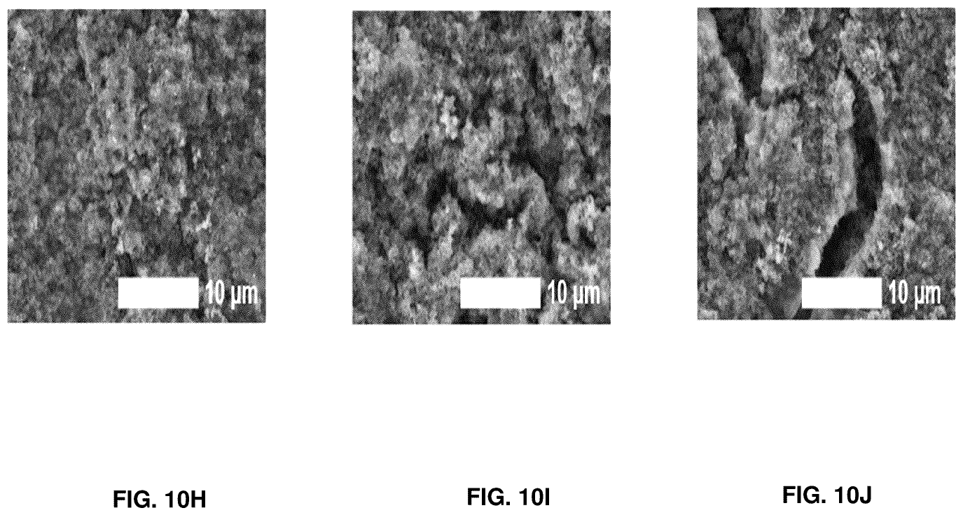

FIGS. 10A-10J show bending test results for 100, 500, 1000 cycles for the nitrate sensor tested continuously after bending at each concentration for 5 minutes, with increasing number of cycles the OCP increases (FIG. 10A), calibration curves for nitrate sensor (FIG. 10B), temporal response of pH sensor in different pH levels of water, Single test lasted for 5 minutes at each concentration (FIG. 10C), calibration curves for bending cycles showing minimum variation between the sensor responses (FIG. 10D), SEM images for nitrate sensor surface 100 cycles (FIG. 10E), 500 cycles (FIG. 10F), bubble formation on nitrate sensor surface, SEM images for pH sensor surface (FIG. 10G), 100 cycles (FIG. 10H), 500 cycles (FIG. 10I), appearance of crack on pH sensor surface (FIG. 10J).

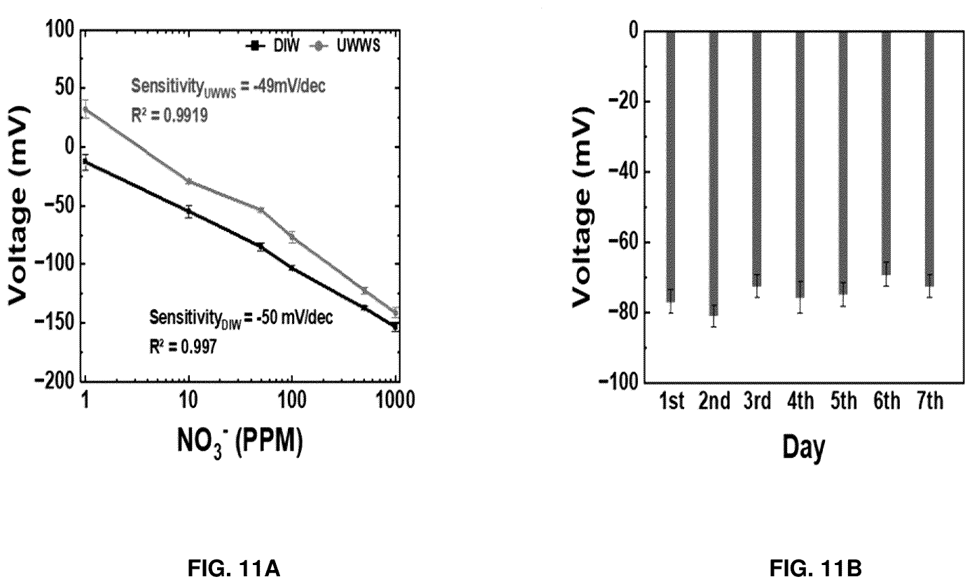

FIGS. 11A-11B show a comparison of calibration plots of the nitrate sensor in DIW and USWS (FIG. 11A). The open circuit voltage was obtained from the average values and the error bars represent standard error for three repeated measurements. The time stability test was done in USDA water sample containing 100 ppm of nitrate for 7 days (FIG. 11B).

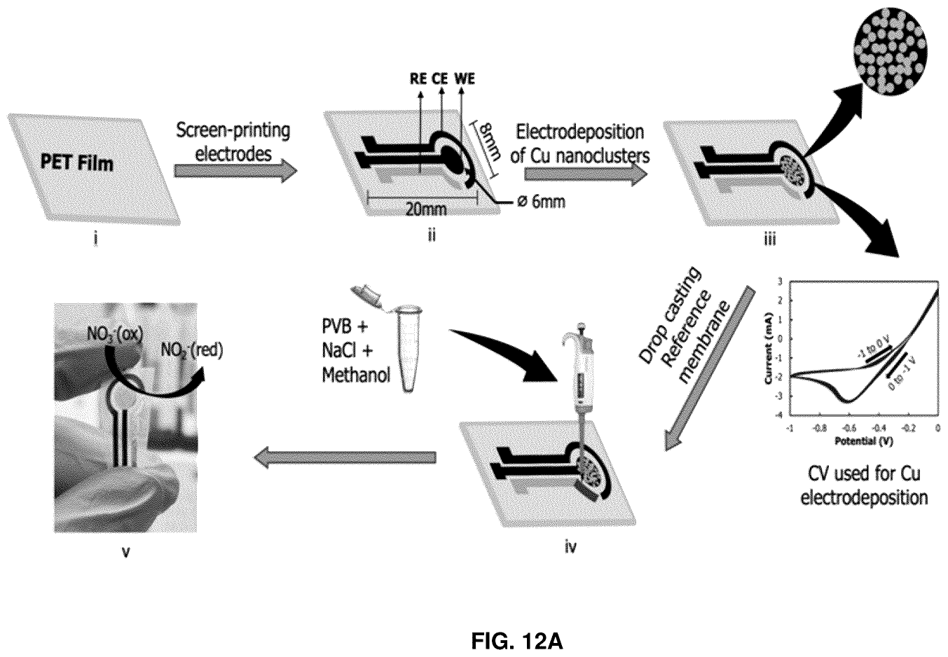

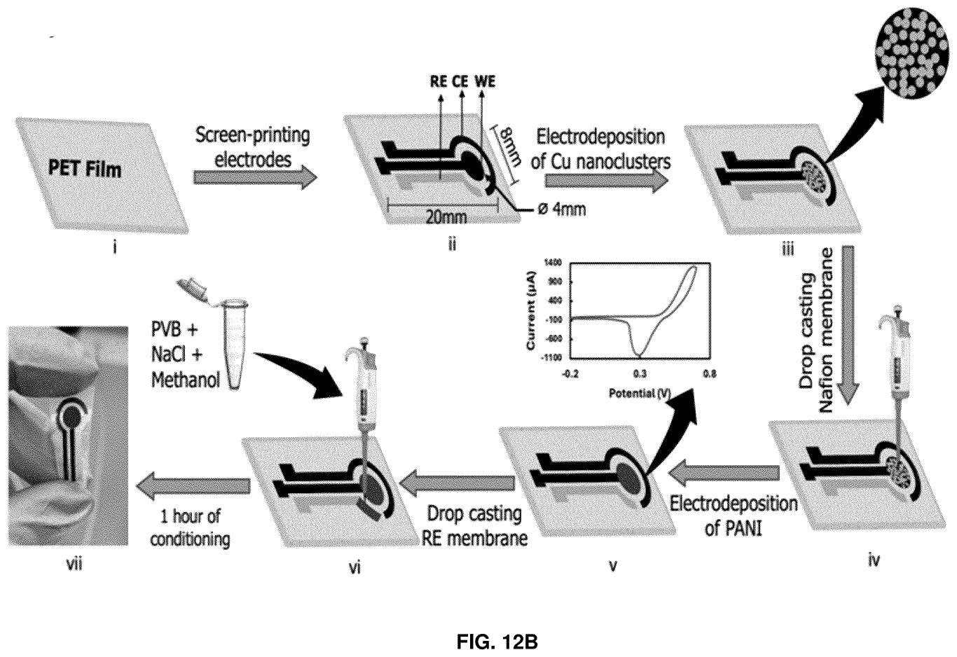

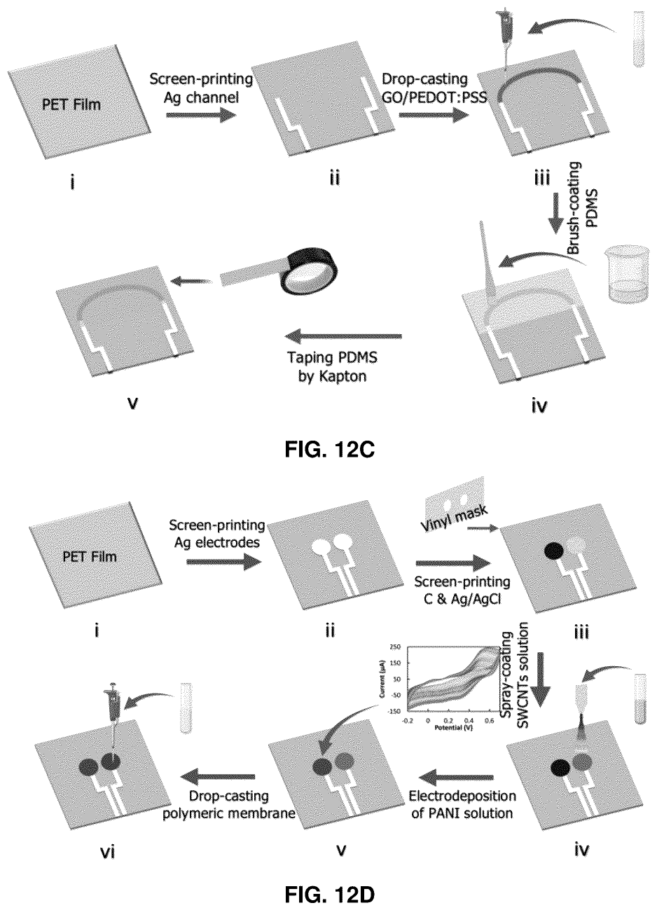

FIGS. 12A-12D show fabrication steps for nitrate sensor where on top of a PET film (i) WE, CE, and RE were screen-printed (ii), Cu electrodepositing on top of WE (iii), drop-casting polymeric membrane on top of RE (iv), and a micrograph of a nitrate sensor (v) (FIG. 12A). Fabrication steps for ammonium sensor where on top of a PET film (i) WE, CE, and RE were screen-printed (ii), Cu electrodepositing on top of WE (iii), drop-casting Nafion on top of WE (iv), electrodepositing PANI on top of WE (v), drop-casting polymeric membrane on top of RE (vi), and a micrograph of an ammonium sensor (vii) (FIG. 12B). Fabrication steps for the temperature sensor where on a PET film (i) Ag channels were screen-printed (ii), a nanocomposite of GO and PEDOT:PSS was drop-casted connecting the Ag channels (iii), Brush-coating of PDMS over the nanocomposite (iv), Kapton tape was used on top of PDMS layer for encapsulation (v) (FIG. 12C). Fabrication steps for the pH sensor where on top of a PET film (i) WE and RE were screen-printed (ii), C and Ag/AgCl ink were screen-printed on top of WE and RE respectively (iii), SWCNTs were spray deposited on top of RE (iv), PANI was electrodeposited on top of WE (v), and Polymeric membrane was drop-casted on top of RE (vi) (FIG. 12D).

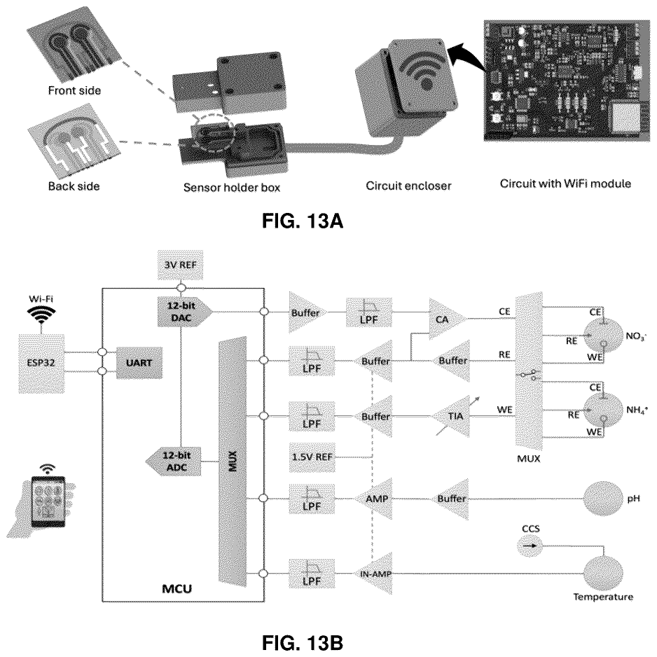

FIGS. 13A-13B shows a multi-parametric water sensor with a first surface of a substrate substrate showing nitrate (left) and ammonium (right) sensors, back side of the substrate showing temperature sensor (half circle) and pH sensor (2 electrodes). The substrate containing the sensors is housed inside the enclosure which is connected to a circuit/communication system enclosure containing the whole circuit including the WiFi module (FIG. 13A). The system-level diagram of the data acquisition and communication circuit (FIG. 13B).

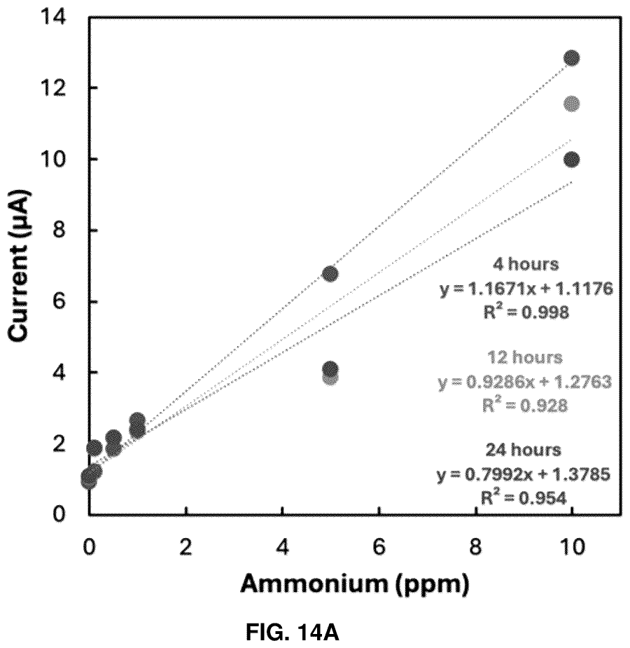

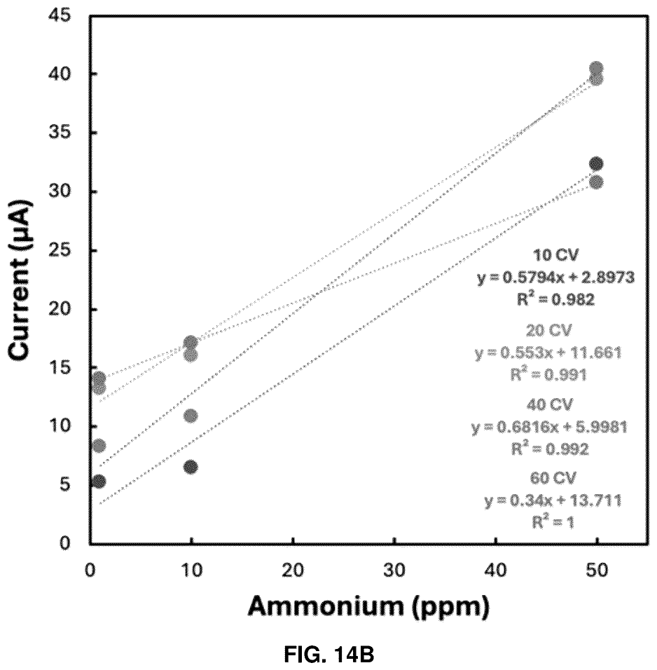

FIGS. 14A-14B show conditions used to prepare PANI for ammonium sensor preparation, including mixing time of PANI solution (FIG. 14A) and CV cycle for PANI electrodeposition (FIG. 14B).

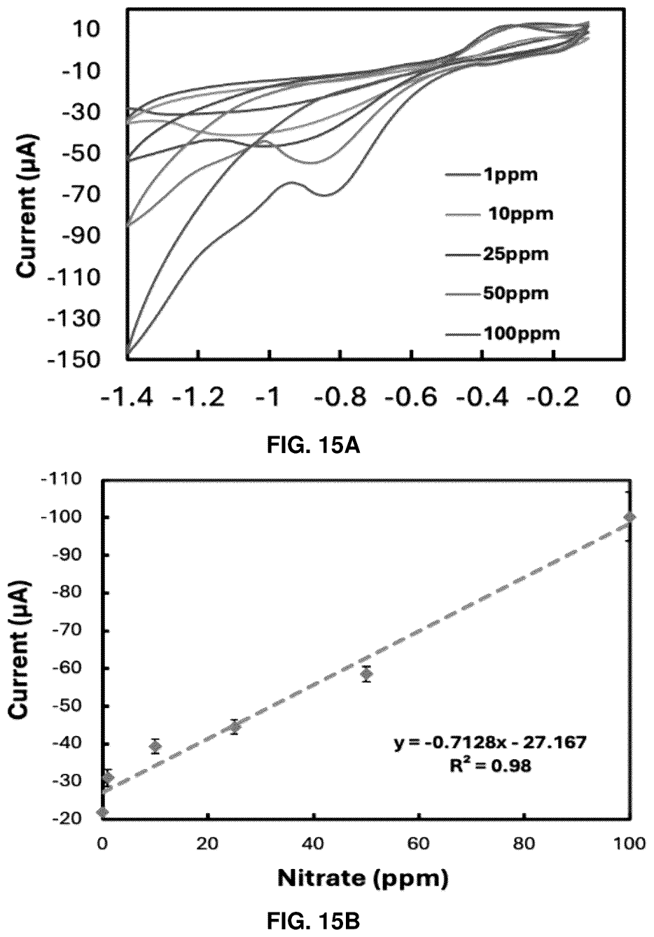

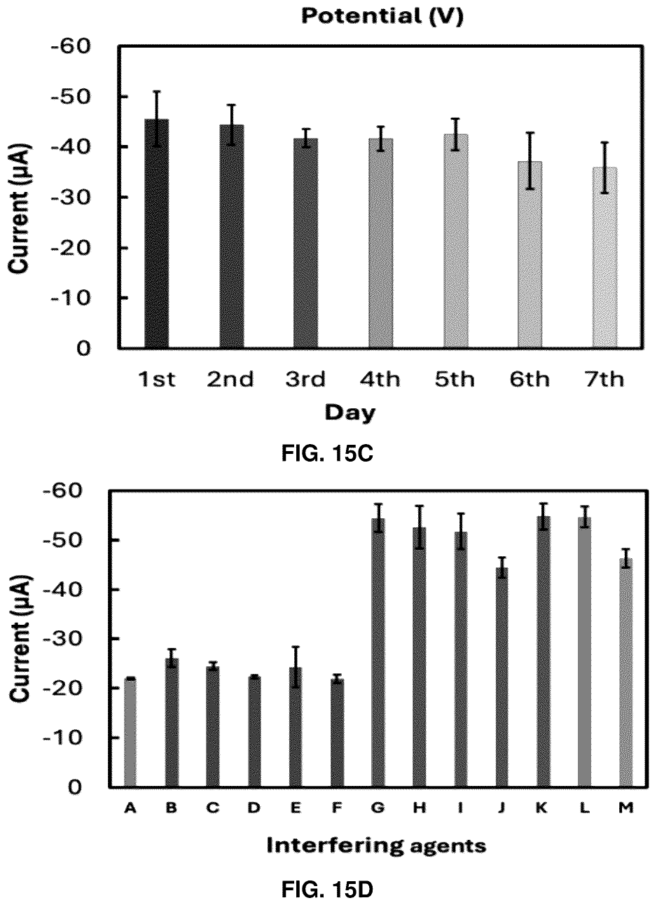

FIGS. 15A-15D show CV at different concentrations of nitrate (FIG. 15A), a calibration plot of a nitrate sensor where the highest reduction peak versus nitrate concentration is shown (FIG. 15B), a stability test of nitrate sensor for a week (FIG. 15C), a selectivity test of nitrate sensor (FIG. 15D) (A=Blank, B═NO₂—, C═Cl⁻, D=PO₄³⁻, E=NH⁴⁺, F═SO₄²⁻, G=NO₂⁻+NO₃⁻, H=Cl⁻+NO₃⁻, I=PO₄³⁻+NO₃⁻, J=NH₄⁺+NO₃⁻, K=SO₄²⁻+NO₃⁻, L=NO₃⁻, M=all).

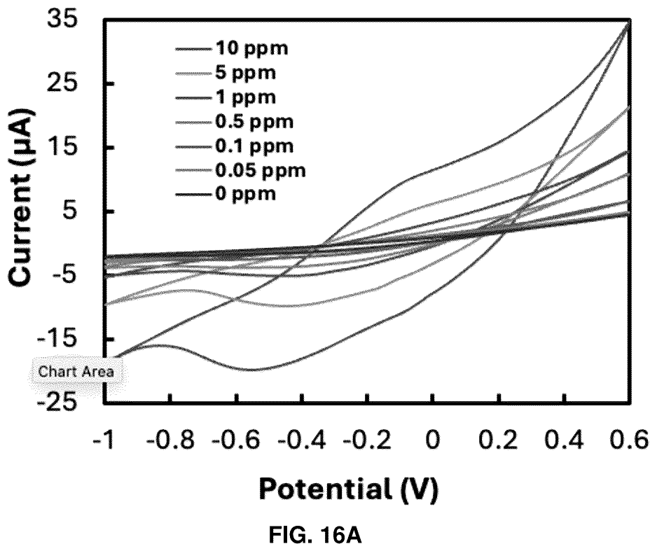

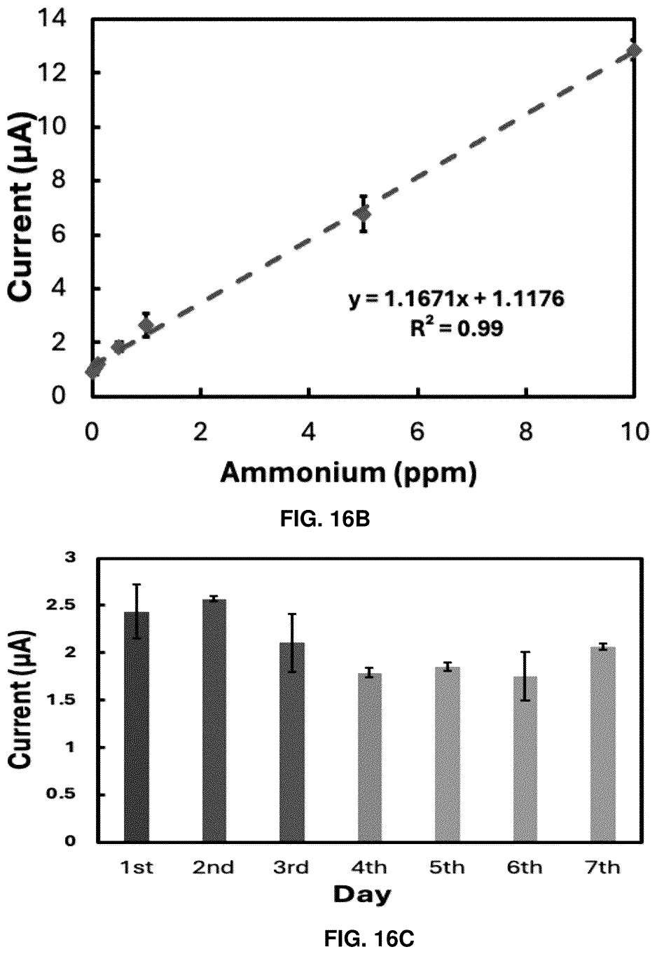

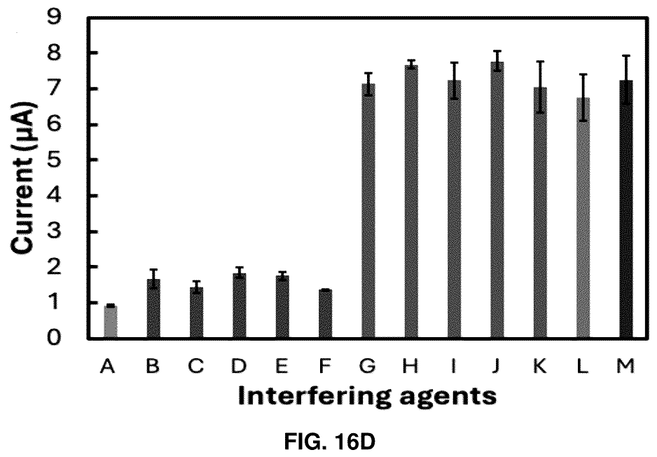

FIGS. 16A-16D show CV at different concentrations of NH₄⁺ (FIG. 16A), calibration plot of NH₄⁺ sensor where the highest reduction peak versus NH₄⁺ concentration is shown (FIG. 16B), stability test of NH₄⁺ sensor for a week (FIG. 16C), and selectivity test of NH₄⁺ sensor (FIG. 16D) (A=Blank, B═Cl—, C═NO3-, D=NO2-, E=SO42-, F═PO43-, G=Cl—+NH4+, H═NO3-+NH4+, I=NO2-+NH4+, J=SO42-+NH4+, K═PO43-+NH4+, L=NH4+, M=All).

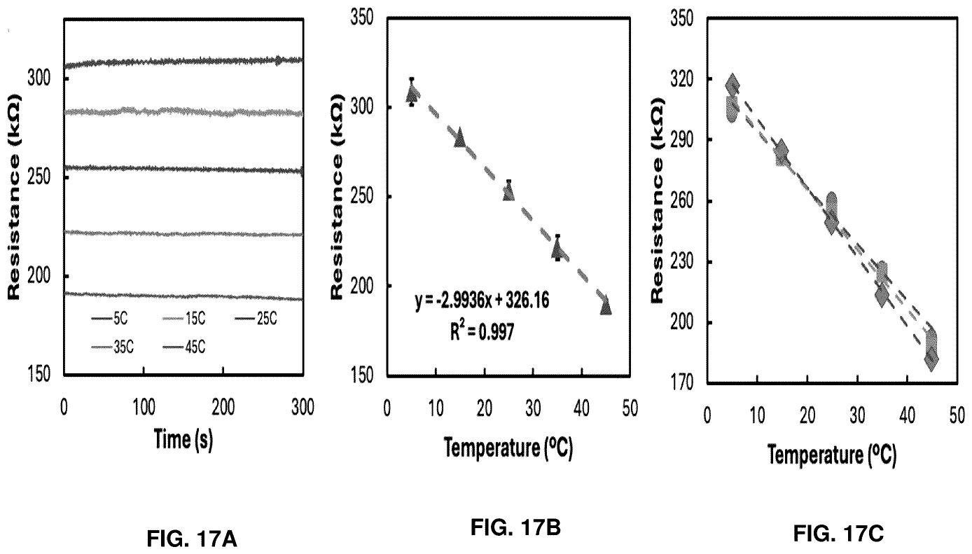

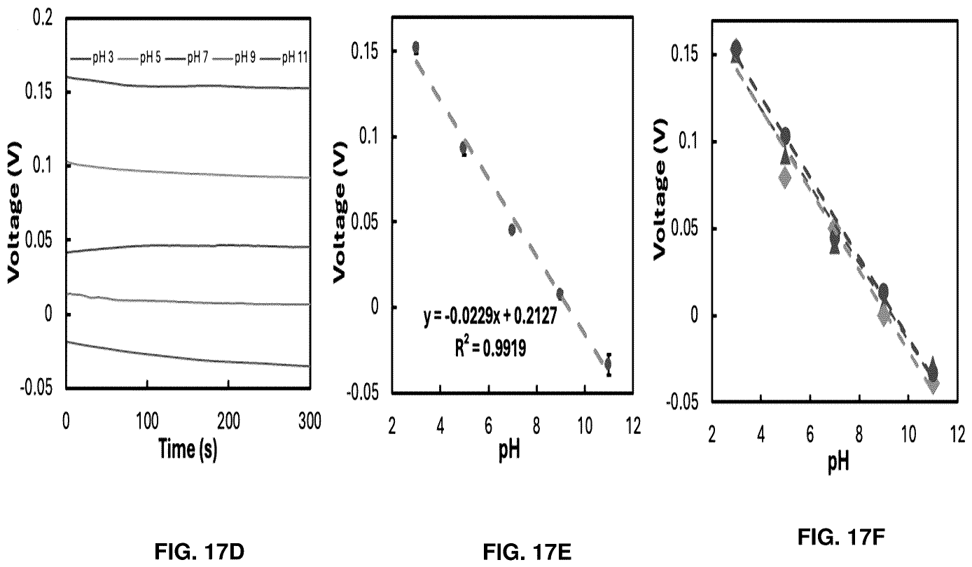

FIGS. 17A-17F show the dynamic response of the Temperature (T) sensor over a temperature range from 5 to 45° C. (FIG. 17A), calibration plot for the T sensor, with error bars derived from three measurements at each temperature level (FIG. 17B), reproducibility test, showing the calibration plot of three different T sensors (FIG. 17C), the dynamic response of the pH sensor over a pH range from 3 to 11 (FIG. 17D), calibration plot for the pH sensor, with error bars derived from three measurements at each pH level (FIG. 17E), and reproducibility test, showing the calibration plot of three different pH sensors (FIG. 17F).

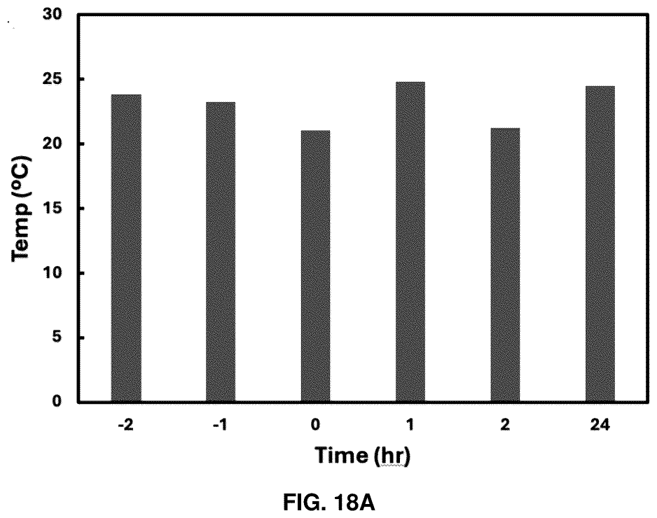

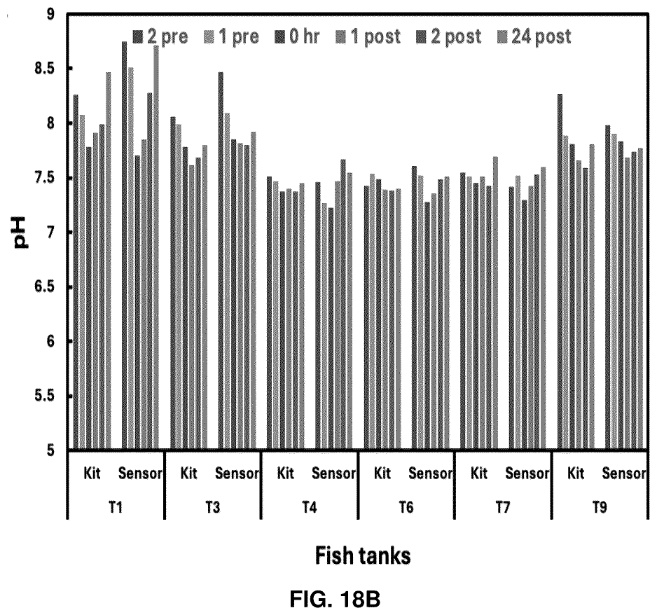

FIGS. 18A-18B show temperature measurement of USDA fish tank (T1) with T sensor (FIG. 18A). A comparison is shown between pH kit and developed pH sensor for all USDA Fish tanks (T1, T3, T4, T6, T7 and T9) in different time period. Here 2 pre=2 hours before ammonium dosing, 1pre=1 hour before ammonium dosing, 0 hr-after dosing, 1post=1 hour after dosing, 2post=2 hours after dosing (FIG. 18B).

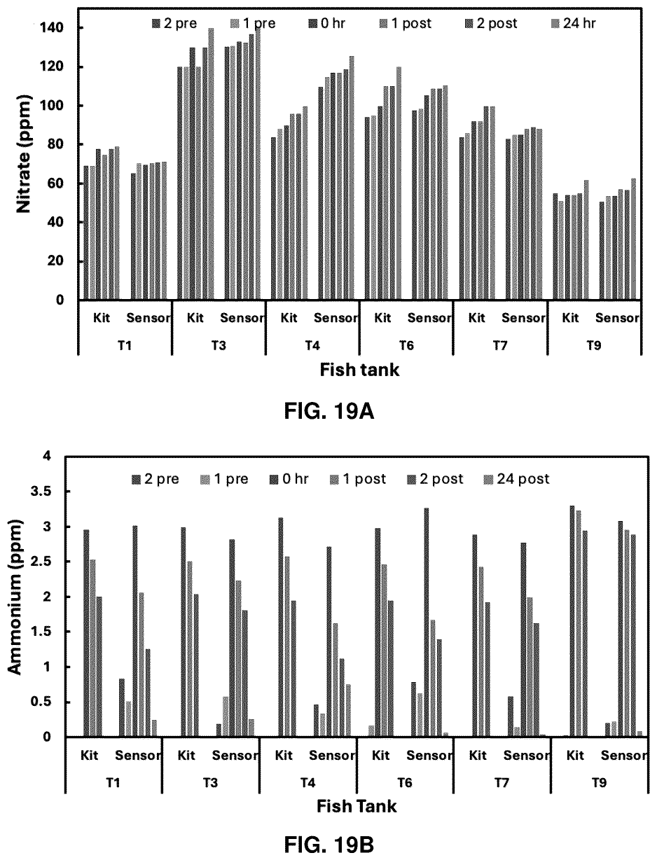

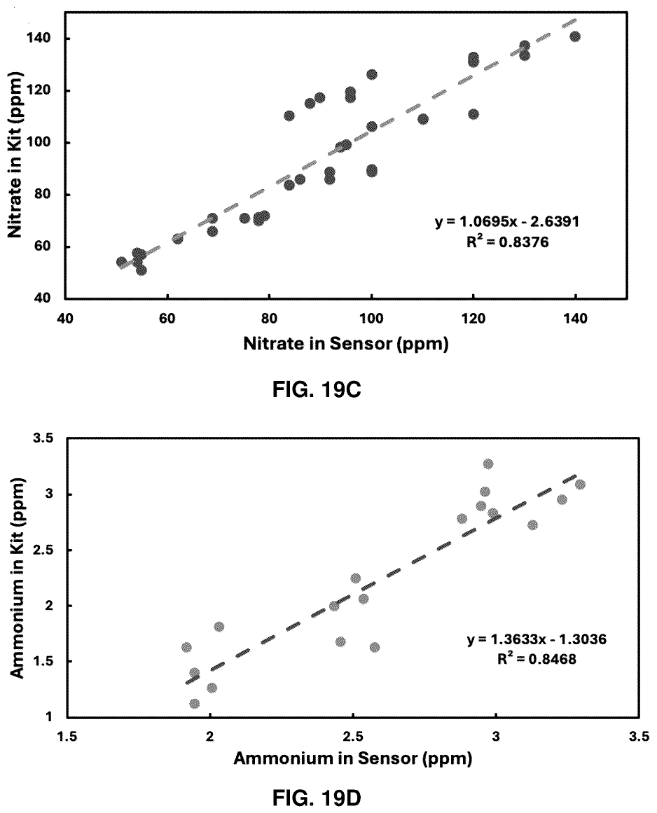

FIGS. 19A-19D show plots comparing the fabricated nitrate sensor with the commercial nitrate measurement kit (FIG. 19A) and the fabricated ammonium sensor with the commercial ammonium measurement kit (FIG. 19B) showing hourly variations in nitrate concentrations for all USDA Fish tanks (T1, T3, T4, T6, T7 and T9) in different time periods. Here 2 pre=2 hours before ammonium dosing, 1pre=1 hour before ammonium dosing, 0 hr=after dosing, 1post=1 hour after dosing, 2 post=2 hours after dosing. Scatter plots of nitrate (FIG. 19C) and ammonium values (FIG. 19D) measured with the sensors against the values obtained from commercial kits.

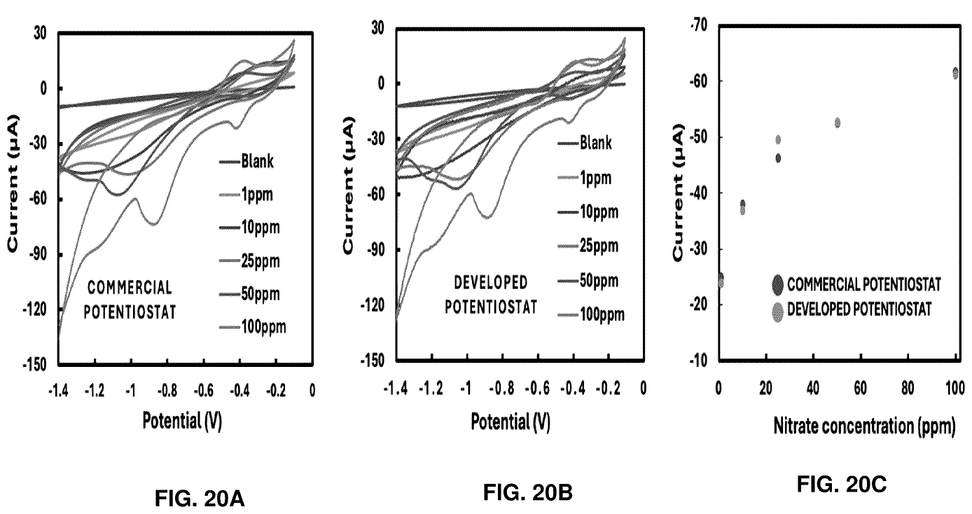

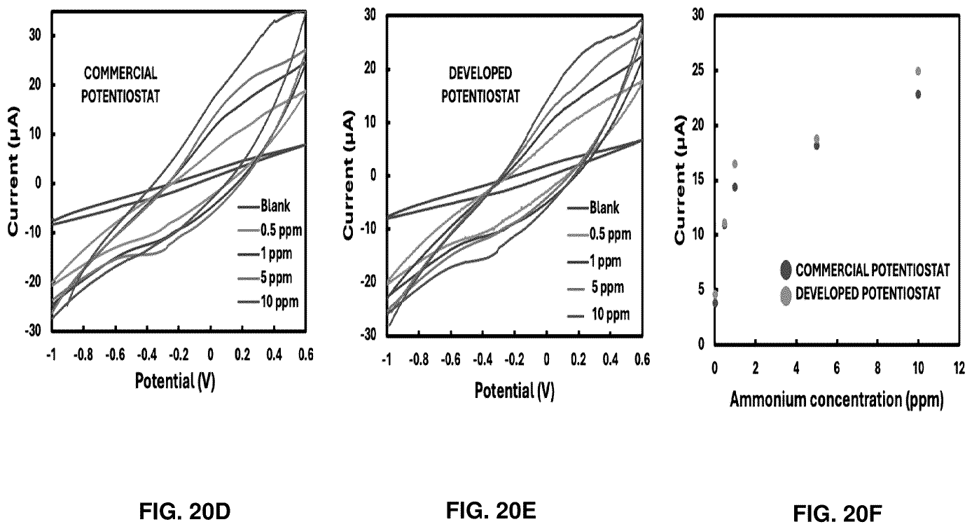

FIGS. 20A-20F show a comparison between commercial and tested potentiostat where CVs were performed for different nitrate concentration through commercial potentiostat (FIG. 20A) and tested potentiostat (FIG. 20B) and plotted all the reduction peak current obtained from different nitrate concentrations to compare between two potentiostats (FIG. 20C). CVs performed for different ammonium concentrations through commercial potentiostat (FIG. 20D) and the tested potentiostat (FIG. 20E) and plotted all the oxidation peak current obtained from different ammonium concentrations to compare between two potentiostats (FIG. 20F).

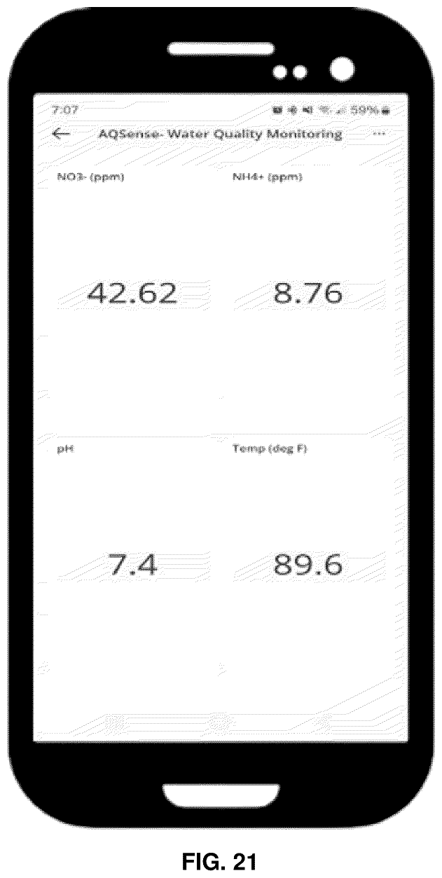

FIG. 21 shows an exemplary display device providing remote access to unknown concentrations of nitrate and ammonia, as well as pH and temperature readings via a display.

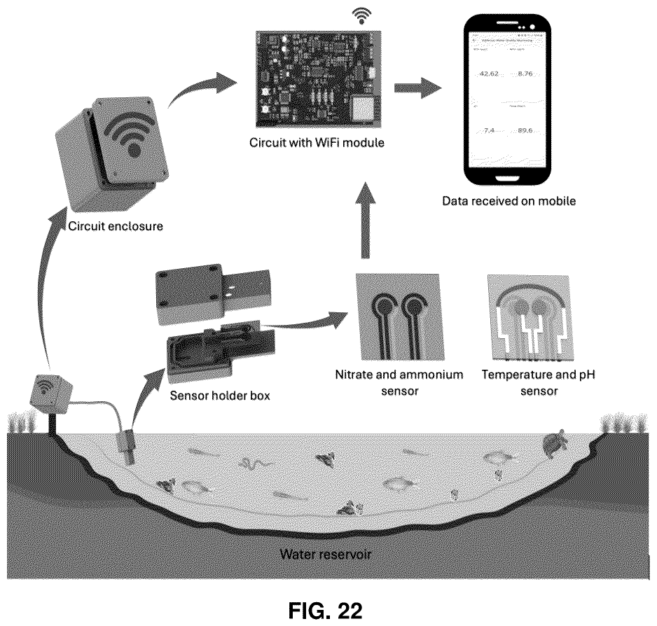

FIG. 22 shows an illustration of in situ analyte measurements using an exemplary multi-parametric water sensor.

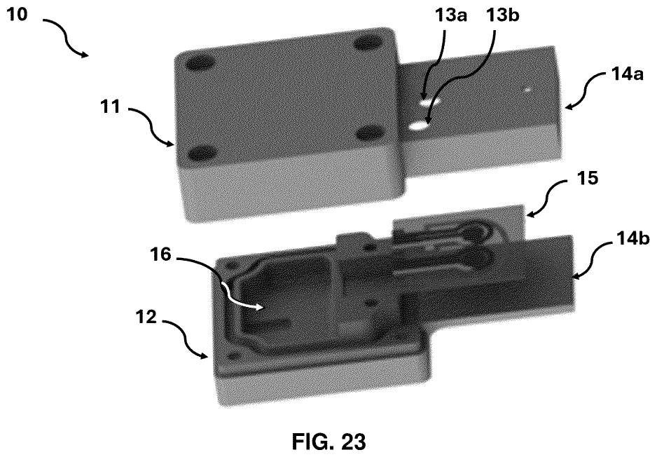

FIG. 23 shows an exemplary enclosure a the multi-parametric water sensor.

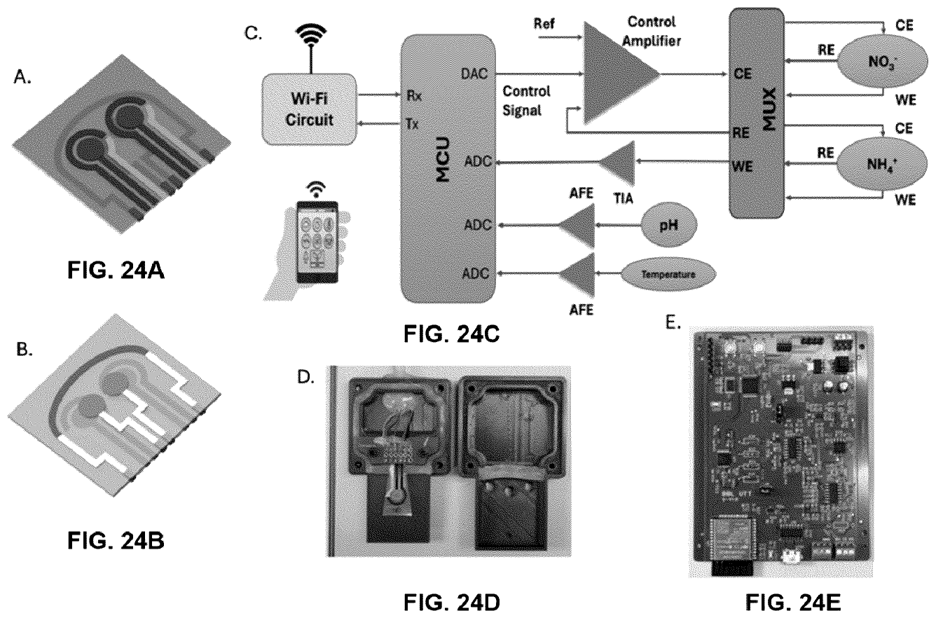

FIGS. 24A-24E shows a first side of the PET substrate showing nitrate (left) and ammonium (right) sensor, (FIG. 24A), a second surface of the PET substrate showing T sensor (half circle) and pH sensor (2 electrodes), (FIG. 24B) The system diagram of the data acquisition circuit, (FIG. 24C), an enclosure holding the sensor and the circuit (FIG. 24D), and a micrograph of the entire circuit including WiFi module (FIG. 24E).

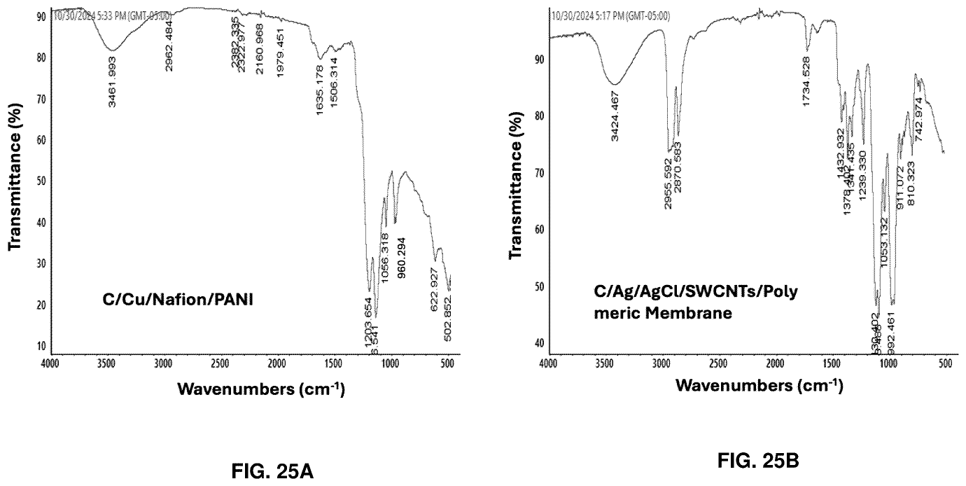

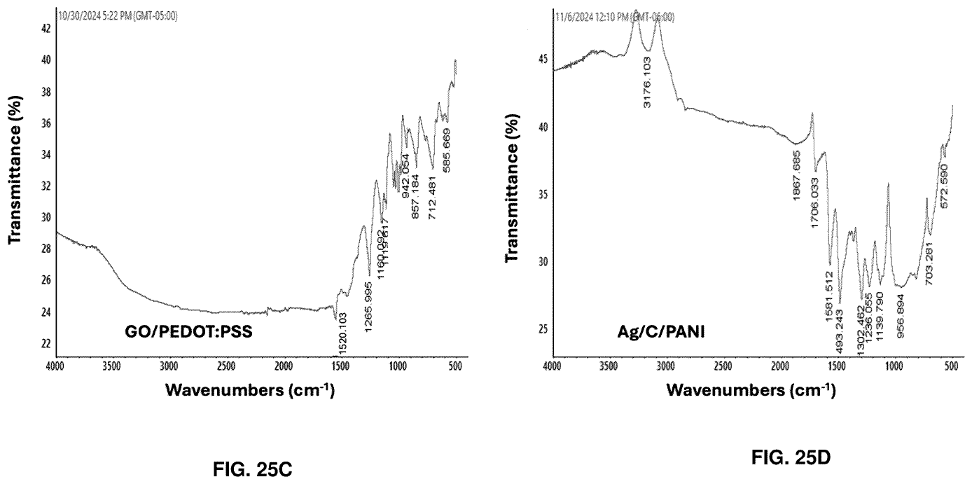

FIGS. 25A-25D show FT-IR characterization of different sensor surfaces. PANI was electrodeposited on top of the WE of the ammonium sensor (FIG. 25A), polymeric membrane on top of the RE (FIG. 25B), a composite of GO and PEDOT:PSS in the T sensor (FIG. 25C), PANI was electrodeposited on top of the WE of pH sensor (FIG. 25D).

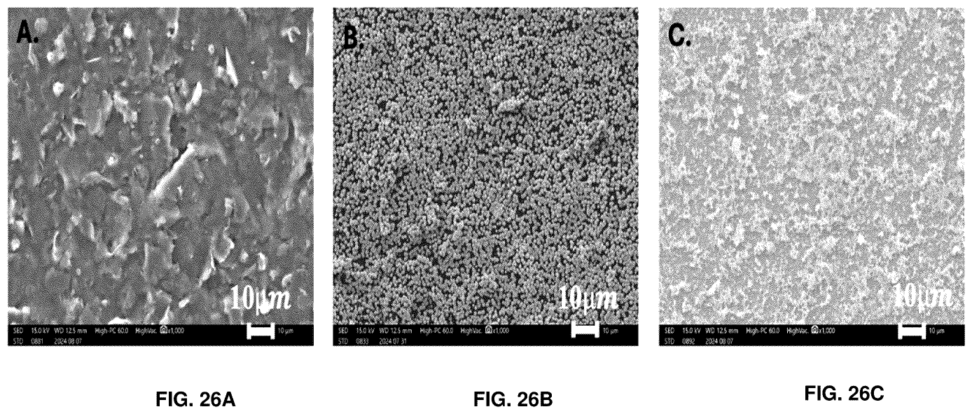

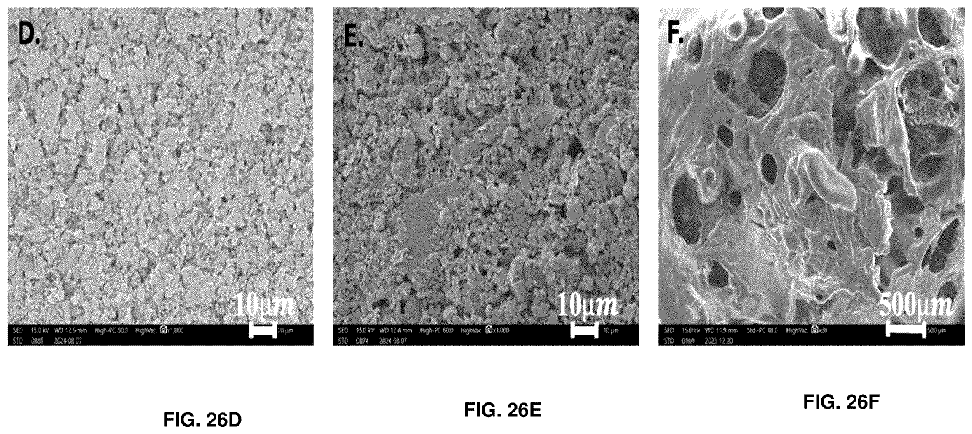

FIGS. 26A-26F show SEM images of screen-printed C electrode (FIG. 26A), homogenous distribution of Cu nanoclusters on top of C electrode (FIG. 26B), uniform distribution of PANI on top of Cu/C electrode (FIG. 26C), screen-printed Ag electrode (FIG. 26D), screen-printed Ag/AgCl ink on top of Ag electrode (FIG. 26E), distribution of polymeric membrane on top of Ag/AgCl electrode (FIG. 26F).

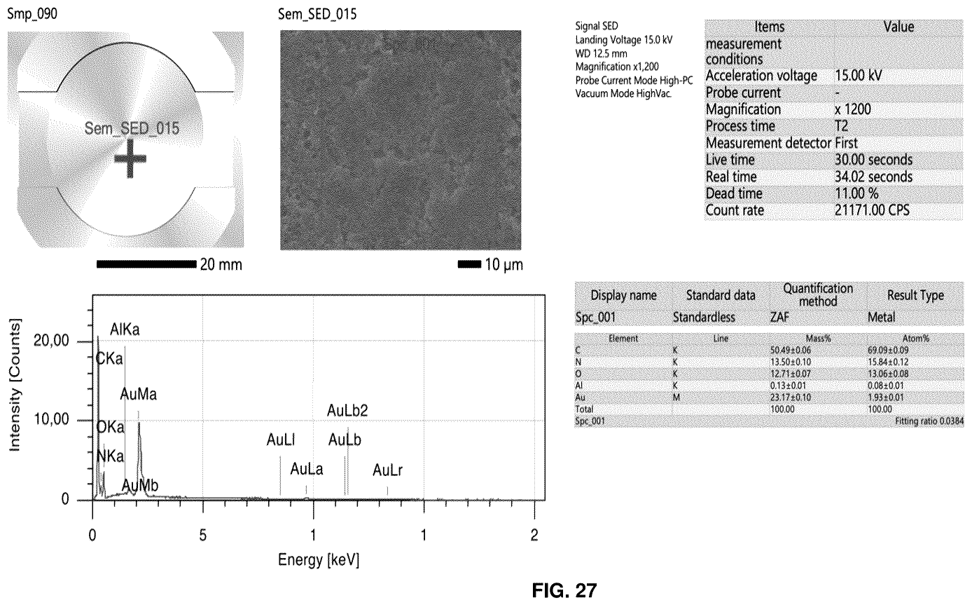

FIG. 27 shows EDS analysis of ammonium sensor where PANI was electrodeposited on top of Au coated electrode.

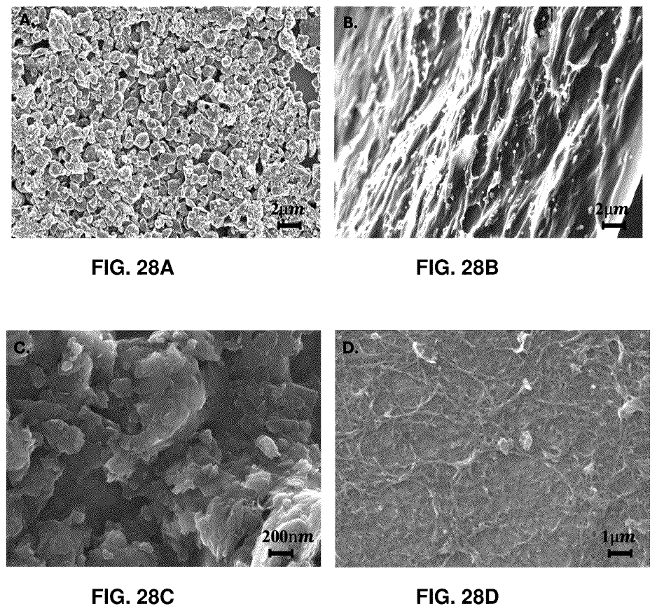

FIGS. 28A-28D show SEM images of homogenously distributed GO (FIG. 28A), porous structure of PEDOT:PSS (FIG. 28B), flex-like distribution of GO and PEDOT:PSS composite (FIG. 28C), a network of spray-deposited SWCNTs (FIG. 28D).

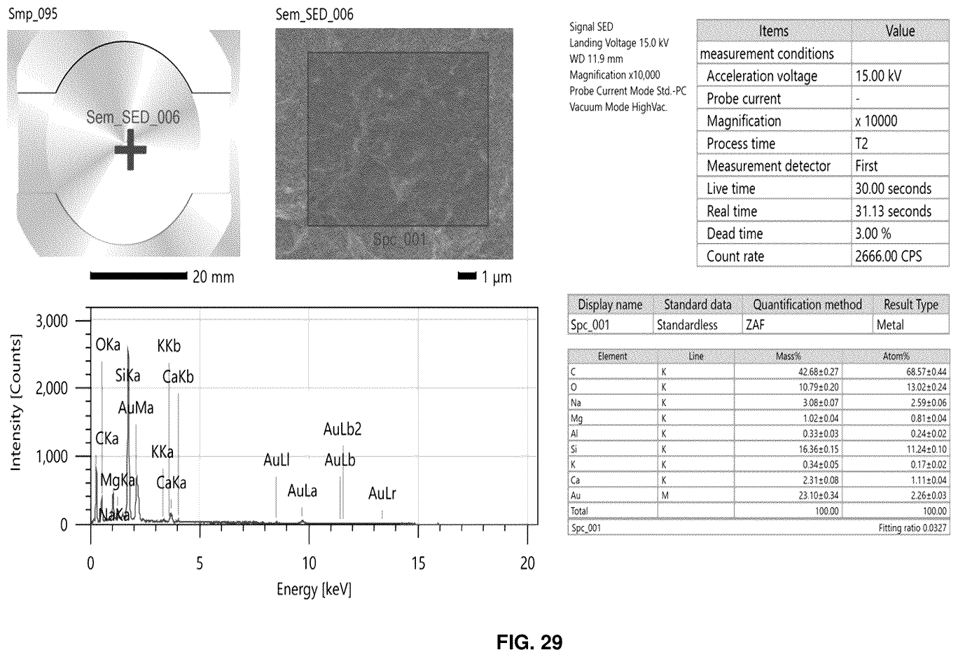

FIG. 29 shows EDS analysis of RE where SWCNTs were spray deposited on top of an Au-coated electrode.

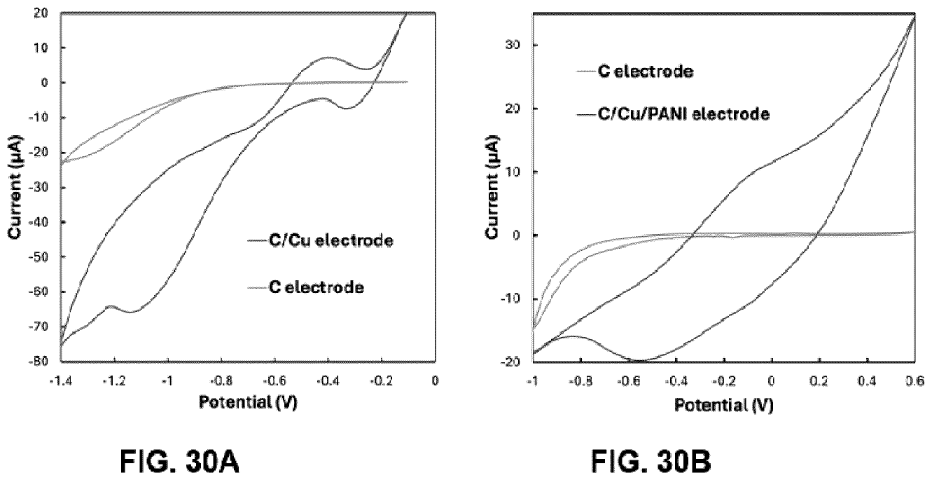

FIGS. 30A-30B show a CV plot for the characterization of nitrate and ammonium sensors. CV plot showing the reduction peak of nitrate detection with and without the presence of Cu (FIG. 30A). CV plot showing the oxidation peak of ammonium with and without the presence of Cu/PANI electrodeposition (FIG. 30B).

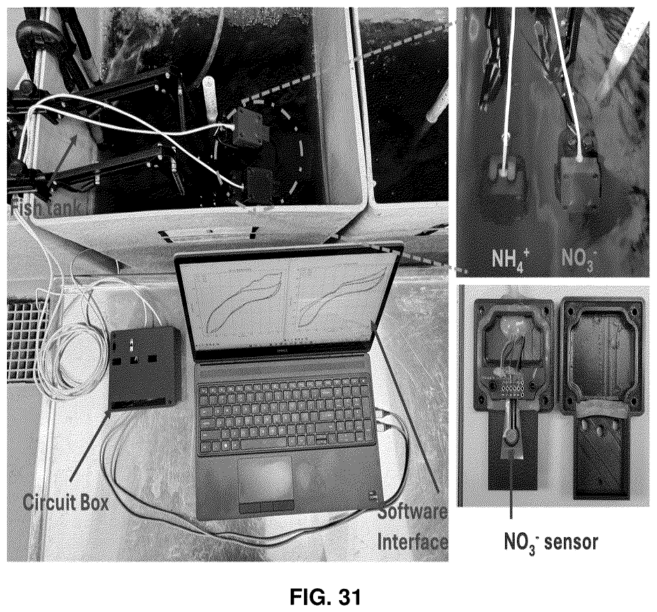

FIG. 31 shows real-time monitoring of nitrate and ammonium in a catfish RAS, where two boxes were used separately to detect nitrate and ammonium. The boxes are wired to the circuit enclosure connected to the PC. The inset shows two boxes submerged in the fish tank and an open box showing the nitrate sensor.

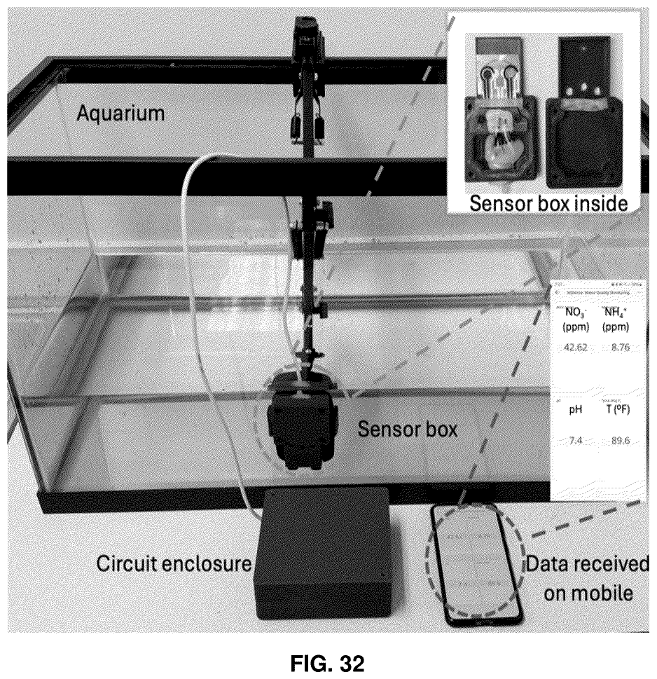

FIG. 32 shows real-time monitoring of nitrate and ammonium in a test setup (aquarium), where the sensor box submerged in water is wired to the circuit enclosure, and the data is received on mobile. The insets show the box interior containing the sensors and the mobile app screenshot.

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” or “consisting of”′ would find direct support due to this definition for any elements disclosed throughout this disclosure. Based on this definition, any element disclosed herein or incorporated by reference may be included in or excluded from the claimed invention.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

A “processor” means one or more microprocessors, central processing units (CPUs), processing circuitry, computing devices, one or more microcontrollers, digital signal processors, or like devices or any combination thereof, regardless of the architecture (e.g., chip-level multiprocessing/multi-core, RISC, CISC, Microprocessor without Interlocked Pipeline Stages, pipelining configuration, simultaneous multithreading). In some embodiments, the processor is operatively connected to memory. The processor and memory may be connected externally or internally.

“Bioagent” means a biological substance, including, but not limited to peptides, enzymes, polypeptides and proteins, nucleotides/nucleic acid, or polynucleotides, any organism, cell, or virus, living or dead, or a nucleic acid derived from such an organism, cell or virus.

“Computer-readable medium” means any medium, a plurality of the same, or a combination of different media, that participate in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include random access memory (RAM) or dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, SecureDigital (SD™) memory card, USB Flash Drives, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

### Multi-Parametric Water Sensor

In one aspect, the disclosure provides a multi-parametric water sensor, comprising a substrate; a sensor array; and an enclosure comprising a water impermeable material. In some aspects, sensor array comprises an analyte sensor comprising a working electrode (WE) and a reference electrode (RE) disposed on a first surface of the substrate; a pH sensor comprising a working electrode (WE) and a reference electrode (RE) disposed on the first surface or a second surface of the substrate; and a temperature sensor disposed on the first surface or a second surface of the substrate. In some aspects, the analyte is an ion, a dissolved gas, or a combination thereof.

In some aspects, the analyte sensor is a potentiometric sensor. In some aspects, the analyte sensor is an ammonia sensor or a nitrate sensor. In some aspects, the multi-parametric water sensor comprises both an ammonia sensor and a nitrate sensor. In some aspects, the ammonia sensor measures total ammonia, wherein the total ammonia is the sum of ammonium (NH₄⁺) and ammonia (NH₃) in solution. In some aspects, the analyte sensor is configured to resist biofouling.

In some aspects, the multi-parametric water sensor comprises two analyte sensors. In some aspects, the two analyte sensors are disposed on the first surface of the substrate. In some aspects, the two analyte sensors are configured to resist biofouling. In some aspects, the pH sensor and the temperature sensor are disposed on the second surface of the substrate. In some aspects, a first analyte sensor comprises a nitrate sensor and a second analyte sensor comprises an ammonia sensor.

In some aspects, the multi-parametric water sensor comprises a sensor array with a coefficient of variance between calibration curves of not more than 3% based on four repeating measurements. In some embodiments, the multi-parametric water sensor comprises a sensor array that has a coefficient of variance between calibration curves of not more than 5% based on four repeating measurements. In some aspects, the analyte sensor has an analyte detection range of about 0.7 ppm or more, about 0.8 ppm or more, about 0.7 ppm to about 100 ppm, or about 0.8 to about 100 ppm. In some aspects, the nitrate sensor has an analyte detection range of about 0.8 ppm to about 100 ppm. In some aspects, the ammonium sensor has an analyte detection range of about 0.7 ppm to about 10 ppm.

In some aspects, the analyte sensor has a coefficient of variance between measurements of not more than about 10%, not more than about 8%, not more than about 7%, not more than about 6%, not more than about 5%, not more than about 4%, not more than about 2%, or not more than about 1%. In some aspects, the first analyte sensor, the second analyte sensor, or both has a coefficient of variance between measurements of not more than about 10%, not more than about 8%, not more than about 7%, not more than about 6%, not more than about 5%, not more than about 4%, not more than about 2%, or not more than about 1%. In some aspects, the analyte sensor has a coefficient of variance between measurements of not more than about 10%, about 9%, about 8%, or about 5% before and after a dynamic folding test, wherein in the dynamic folding test the flexible plant sensor in an unbent orientation is bent to a 90° angle, returned to the unbent orientation, and repeated up to 100 cycles, 500 or 1000 cycles. In some aspects, the first analyte sensor, the second analyte sensor, or both has a coefficient of variance between measurements of not more than about 10%, about 9%, about 8%, or about 5% before and after a dynamic folding test, wherein in the dynamic folding test the flexible plant sensor in an unbent orientation is bent to a 90° angle, returned to the unbent orientation, and repeated up to 100 cycles, 500 or 1000 cycles.

In some embodiments, the analyte sensor has a coefficient of variance of <1%, <2%, <3%, <4%, or <5% up to one hour, or <1%, <2%, <3%, <4%, or <5% up to 7 days.

In some embodiments, the first analyte sensor, the second analyte sensor, or both has a coefficient of variance of <1%, <2%, <3%, <4%, or <5% up to one hour, or <1%, <2%, <3%, <4%, or <5% up to 7 days.

In one aspect, the disclosure provides a multi-parametric water sensor, comprising a substrate; a sensor array; and an enclosure comprising a water impermeable material. In some aspects, the sensor array comprises a first analyte sensor comprising a working electrode (WE), a counter electrode (CE), and a reference electrode (RE) disposed on a first surface of the substrate; a second analyte sensor comprising a working electrode (WE), a counter electrode (CE), and a reference electrode (RE) disposed on the first surface of the substrate; a pH sensor comprising a working electrode (WE) and a reference electrode (RE) disposed on a second surface of the substrate; and a temperature sensor disposed on the second surface of the substrate. In some aspects, the enclosure comprises a sensor section and a water permeable section. In some aspects, the analyte sensor RE and the pH sensor RE is the same RE electrode. In some aspects, the RE is in communication with both the analyte sensor and the pH sensor.

In some aspects, the first analyte sensor is a nitrate sensor and the second analyte sensor is an ammonium sensor. In some aspects, the first analyte sensor and the second analyte sensor are configured to resist biofouling.

In some aspects, the multi-parametric water sensor comprises a data acquisition system (DAS), wherein the DAS comprises a processor; a communication unit; a memory unit; and a power supply unit, and wherein the data acquisition system is in communication with the sensor array. In some aspects, the memory unit is communicatively coupled to the processor, the memory unit having stored thereon computer software comprising a set of instructions that, when executed by the processor, causes the data acquisition unit to receive sensor data from the sensor array; and send, via the communication unit, the sensor data to an external device. In some aspects, the memory unit comprises a non-transitory computer-readable medium. In some aspects, the sensor data are sent, via the communication unit, to an Internet of Things (IOT) cloud server configured to interact with one or more IoT-capable devices. In some aspects, the DAS comprises a microcontroller unit. In some aspects, the instructions comprise a signal calibration of at least one electrode. In some aspects, the instructions comprise a pH-based signal correction, a temperature-based signal correction, or a combination thereof.

In some embodiments, the data acquisition system further comprises a voltage booster. In certain embodiments, the data acquisition system may include one or more processors and memory, which may be coupled together with a bus. The one or more processors and other components may be coupled together with a bus, a separate bus, or may be directly connected together or coupled using a combination of the foregoing. The memory may contain executable code or software instructions that when executed by the one or more processors or processing circuitry cause the one or more processors or processing circuitry to perform the techniques disclosed herein. The memory may be configured to store the one or more calibration plots and/or other instructions.

In some aspects, the data are sent via a wired connection. In some aspects, the data are sent via a wireless connection. In some embodiments, the communication module implements a communication protocol based on Bluetooth or Bluetooth low energy transmission, Wi-Fi, Wi-Max, IEEE 802.11 technology, a radio frequency (RF) communication. In some embodiments, the communication module implements a communication protocol based on general packet radio service (GPRS), enhanced data GSM environment (EDGE), long term evolution-advanced (LTE-A), LTE, 3G, 4G, 5G, code division multiple access (CDMA), wideband CDMA (WCDMA), evolution-data optimized (EVDO), wireless broadband Internet (Wibro), Mobile WiMax, Digital Enhanced Cordless Telecommunications (DECT), Digital AMPS (IS-136/TDMA, Integrated Digital Enhance Network (iDEN), HSPA+, Flash-OFDM, HIPERMAN, WiFi, IBurst, UMTS, W-CDMA, HSPDA+HSUPA, UMTS-TDD and other formats for utilizing cell phone technology, telephony antenna distributions and/or any combinations thereof, and including the use of satellite, microwave technology, the internet, cell tower, telephony and/or public switched telephone network lines. In some embodiments, the communication module implements a communication protocol based on near field communication (NFC).

In some aspects, the method includes sending electrode data, via the communication unit, to an Internet of Things (IoT) cloud server configured to interact/communicate with one or more IoT-capable devices. In some aspects, the method includes sending electrode data, via the communication unit, to an external device via a wired connection. In some aspects, the method includes sending electrode data to an external device wirelessly. In some aspects, the external device and/or IoT-capable device may include, but is not limited to, a desktop computer, a laptop computer, typical cell phone, smart device (e.g., smart phones), or similar apparatus including all remote cellular phones using channel access methods defined above (with cellular equipment, public switched telephone network lines, satellite, tower and mesh technology), mobile phones, PDAs, or a television, watch, timepiece or fob watch and other similar apparatus with WIFI and wireless capability, and controllers having internet or wireless connectivity.

### Nitrate Sensor

In some aspects, the nitrate sensor comprises a working electrode (WE) and a reference electrode (RE). In some aspects, the RE is in communication with both a pH sensor and the nitrate sensor. In some aspects, the nitrate sensor RE is not shared or not in communication with a sensor separate from the nitrate sensor. In some aspects, the nitrate sensor WE comprises a poly(3-octyl-thiophene) (POT)-MoS₂ nanocomposite. In some aspects, the ratio of POT to MoS₂ is about 1:1 to about 1:6, about 1:2 to about 1:5, about 1:3 to about 1:5, or about 1:4 to about 1:5. In some aspects, the ratio of POT to MoS₂ is about 1:1, 1:2, 1:3, 1:4, 1:5, or about 1:6.

The analyte sensors according to any of the embodiments described herein are highly selective for target analytes relative to interfering species. For example, the sensors according to any of the embodiments described herein will detect a higher signal for target analytes relative to a signal detected from interfering species alone. In some embodiments, the selectivity of the sensors according to any of the embodiments described herein may be at least 50× higher for target analytes relative to a signal corresponding to interfering species. In some embodiments, the selectivity for target analytes is at least 50×, 40×, 30×, 25×, 20×, 19×, 18×, 17×, 16×, 15×, 14×, 13×, 12×, 11×, 10×, 9×, 8×, 7×, 6×, 5×, 4×, 3×, 2×, 1.5×, 1.4×, 1.3×, 1.2×, or 1.1× higher than a signal for one or more interfering species. The selectivity, i.e., signal, for target analytes may be 1.1× to 50× relative to one or more interfering species. In some embodiments, the selectivity for target analytes is any value within the foregoing ranges.

In some aspects, the nitrate sensor comprises an ion selective membrane (ISM). In some aspects, the (POT)-MoS₂ nanocomposite is immobilized in the ISM. In some aspects, the ISM is selective for nitrate ions. In some aspects, the ISM is uniform, for example disposed as a uniform layer or coated as a uniform layer. In some aspects, the nitrate sensor is selective for nitrate over interring ions. In some aspects, the interring ions comprise Cl⁻, SO₄²⁻, HCO₃⁻, PO₄³⁻, and NO₂⁻, or a combination thereof.

In some aspects, the RE comprises silver. In some aspects, the RE comprises Ag/AgCl. In some aspects, the RE further comprises a single-walled carbon nanotube (SWCNT) layer. In some aspects, the RE further comprises a poly (vinyl butyral) (PVB) coating.

In some aspects, the nitrate sensor comprises a working electrode (WE), a counter electrode (CE), and a reference electrode (RE). In some aspects, the nitrate sensor WE and the CE comprise carbon. In some aspects, the nitrate sensor WE further comprises copper.

In some aspects, the nitrate sensor RE comprises silver. In some aspects, the nitrate sensor RE comprise Ag/AgCl. In some aspects, the nitrate sensor RE further comprises a poly (vinyl butyral) (PVB) coating.

In some aspects, the nitrate sensor CE comprises platinum.

### Ammonium Sensor

In some aspects, the ammonium sensor comprises a working electrode (WE), a counter electrode (CE), and a reference electrode (RE). In some aspects, the ammonium sensor WE and the CE comprise carbon. In some aspects, the ammonium sensor WE further comprises copper. In some aspects, the ammonium sensor WE further comprises a perfluorinated polymer coating comprising a polytetrafluoroethylene (PTFE). In some aspects, the ammonium sensor WE further comprises a thermoplastic resin membrane. In some aspects, the thermoplastic resin is a polyaniline (PANI) resin.

In some aspects, the ammonium sensor RE comprise silver. In some aspects, the ammonium sensor RE comprise Ag/AgCl. In some aspects, the ammonium sensor RE further comprises a poly (vinyl butyral) (PVB) coating.

In some aspects, the ammonium sensor CE comprises platinum.

### pH Sensor

In some aspects, the pH sensor comprises a working electrode (WE) and a reference electrode (RE). In some aspects, the RE is in communication with both the pH sensor and a second sensor, for example the nitrate sensor. In some aspects, the pH sensor RE is not shared or not in communication with a sensor separate from the pH sensor. In some aspects, the pH sensor comprises electrodeposited gold. In some aspects, the pH sensor further comprises a thermoplastic resin membrane. In some aspects, the thermoplastic resin is a polyaniline (PANI) resin. In some aspects, the pH sensor WE and the RE comprise carbon. In some aspects, the pH sensor WE and the RE further comprise silver. In some aspects, the pH sensor WE and the RE further comprise Ag/AgCl.

In some aspects, the pH sensor WE further comprises a thermoplastic resin membrane. In some aspects, the thermoplastic resin is a polyaniline (PANI) resin.

In some aspects, the pH sensor RE further comprises a single-walled carbon nanotube (SWCNT) layer. In some aspects, the pH sensor RE further comprises a poly (vinyl butyral) (PVB) coating.

### Temperature Sensor

In some aspects, the temperature sensor comprises silver. In some aspects, the temperature sensor comprises a graphene oxide-Poly(3,4-ethylenedioxythiophene):polystyrene sulfonate (GO-PEDOT:PSS) composite. In some aspects, the temperature sensor further comprises a polydimethylsiloxane (PDMS) layer. In some aspects, the temperature sensor further comprises a polyimide film.

### Substrate

In some aspects, the substrate is a flexible substrate. In some aspects, the substrate is a thermoplastic and/or thermosetting film. In some aspects, the substrate is a polyimide film, a perfluorinated sulfonic-acid isomer film, sulfonated tetrafluoroethylene fluoropolymer-copolymer film, or a polyethylene terephthalate (PET) film. In certain aspects, the substrate is a polyethylene terephthalate (PET) film.

In some aspects, the substrate has a thickness of about 50 μm to about 500 μm, or about 75 μm to about 400 μm, or about 100 μm to about 350 μm, or about 110 μm to about 250 μm, or about 115 μm to about 200 μm. In some aspects, the substrate has a thickness of about 100 μm, about 110 μm, about 115 μm, about 120 μm, about 125 μm, about 130 μm, about 135 μm, about 140 μm, about 145 μm, or about 150 μm.

### Enclosure

The multi-parametric water sensor includes an enclosure. The enclosure protects the electronic components while allowing the sensors to come into contact with water for real-time analysis. This configuration not only supports robust field use but also enables continuous data transmission to a remote server via wireless connectivity. Integrating these elements makes the sensor platform a flexible tool for environmental monitoring, particularly in aquaculture systems where optimal water quality is crucial for fish health and productivity.

In some aspects, the enclosure is 3D-printed. In some aspects, the enclosure comprises a sensor section and a water permeable section. In some aspects, the enclosure further comprises a buffer zone between the sensor section and the water permeable section. In some aspects, the enclosure further comprises a DAS section, wherein the DAS section separates the DAS from the water. In some aspects, the DAS section comprises a separate enclosure. The DAS section as a separate enclosure can be spatially separated from the multi-parametric water sensor enclosure so as to be placed in a dry environment, for example placed on the ground in close proximity to the water or placed above the water. The DAS section is in communication with the multi-parametric water sensor. In some aspects, the DAS section is connected to the multi-parametric water sensor in the same enclosure. In some aspects, the DAS section is a separate enclosure and is connected to the multi-parametric water sensor via a communication wire. In some aspects the communication wire is several inches, several centimeters, several feet, or several meters. It will be readily appreciated that the communication wire may be any length required to place the DAS section above the water or in a dry environment in close proximity to the water being measured. As shown in FIG. 23, the enclosure 10 may include a top half 11 and a bottom half 12 that together form the enclosure 10. The enclosure may include a water permeable section 14a and a sensor section 14b housing a sensor array 15. The water permeable section 14a may include through holes (13a,13b) allowing water to enter and contact the sensor array 15. In some aspects, the permeable section 14a along with the through holes (13a,13b) may be a buffer section isolating the sensors from direct water flow. In some embodiments, for example shown in FIG. 23, the buffer section may have through holes of about one millimeter to allow water to reach the sensor surface without turbulent water directly striking the sensors. In some aspects, the enclosure may include one through hole, two through holes, of several through holes, for example more than two through holes depending on the application and environment. The enclosure may further include a section separate from the sensor section and water permeable section (14a, 14b). In some aspects, the enclosure comprises a DAS section (16), including for example a data logger, which remains securely enclosed to protect its circuitry from water exposure.

### Methods of Measuring One or More Analytes in an Aqueous Environment

Provided herein is a method of measuring one or more analytes in an aqueous environment, comprising placing the multi-parametric water sensor according to any one of numbered items 1-21 in the aqueous environment; measuring a signal corresponding to one or more analytes in the aqueous environment; measuring at least one environmental parameter comprising (i) measuring a signal corresponding to a pH of the aqueous environment; (ii) measuring a signal corresponding to a temperature of the aqueous environment; or (iii) both i) and ii). In some aspects, the method comprises obtaining as a data output from the multi-parametric water sensor an amount of the one or more analytes in the aqueous environment, wherein the amount of at least one of the analytes is based on the measured value of the at least one environmental parameter of the aqueous environment.

In some aspects, the method comprises applying an electrochemical potential across one or more sensors of the sensor array to prevent or reduce biofouling on the one or more sensors. In some aspects, the environmental parameter comprises measuring a signal corresponding to a pH of the aqueous environment and measuring a signal corresponding to a temperature of the aqueous environment. In some aspects, the method comprises measuring total ammonia in the aqueous environment.

In some aspects, the method further comprises measuring the signal corresponding to the pH of the aqueous environment, measuring the signal corresponding to the temperature of the aqueous environment, and determining the ratio of ammonium (NH₄⁺) to ammonia (NH₃) in the aqueous environment. In some aspects, the method further comprises measuring a signal corresponding to an amount of nitrate in the aqueous environment.

In some aspects, each of the measuring steps comprises continuous measurement. In some aspects, each of the measuring steps comprises measurement every 1 minute to 10 minutes or every 2 minutes to 3 minutes. In some aspects, one or more of the measuring steps, the data output, or both is conducted automatically by the multi-parametric water sensor without user intervention. In some aspects, each of the measuring steps and the data output is conducted automatically by the multi-parametric water sensor without user intervention after a step of activating the multi-parametric water sensor.

In some aspects, the multi-parametric water sensor processes the sensor data from the one or more sensors to produce the data output. In some aspects, the processed data are stored in the memory unit of the multi-parametric water sensor. In some aspects, the processed data are sent, via the communication unit, to the IoT cloud server configured to interact with the one or more IoT-capable devices. In some aspects, the data output is obtained from the multi-parametric water sensor. In some aspects, the data output is obtained from the IoT capable device.

In some aspects, the aqueous environment comprises an environment selected from an aquaculture, a hydroponic system, an aquaponic system, watersheds, streams, ponds, lakes, rivers, water reservoirs, fisheries, or wastewater systems.

In some aspects, provided herein is a method of determining a presence or an amount of ammonia (NH₃) in an aqueous environment based on the amount of total ammonia using the multi-parametric water sensor according to any one or combination of aspects disclosed herein.

In some aspects, the method further comprises determining a presence or an amount of nitrate in the aqueous environment. In some aspects, the aqueous environment comprises an environment selected from an aquaculture, a hydroponic system, an aquaponic system, watersheds, streams, ponds, lakes, rivers, water reservoirs, fisheries, or wastewater systems. In some aspects, the method further comprises reducing or increasing agricultural fertilizer based on the presence or the amount of ammonia (NH₃) in the aqueous environment. In some aspects, the method further comprises reducing or increasing agricultural fertilizer based on the presence or the amount of nitrate in the aqueous environment.

### Methods of Preparing a Multi-Parametric Water Sensor

Provided herein is a method of preparing a multi-parametric water sensor comprising an analyte sensor. In some aspects, the method comprises cutting into a substrate a pH sensor working electrode (WE) pattern, an analyte WE pattern, and a reference electrode (RE) patten between the analyte WE pattern and the pH sensor WE pattern. In some aspects, the method comprises cutting a temperature sensor WE pattern comprising a first pattern and second pattern into the substrate, wherein the analyte sensor WE, the RE, and the pH WE are disposed between the first pattern and the second pattern to prepare patterns for a sensor array. In some aspects, the method comprises screen printing an ink comprising silver on the analyte sensor WE pattern and the temperature sensor WE pattern. In some aspects, the method comprises screen printing an ink comprising carbon on the pH sensor pattern. In some aspects, the method comprises screen printing an ink comprising Ag/AgCl on the RE pattern. In some aspects, the method comprises a sintering step. In some aspects, the method following the sintering step, the method further comprises coating a PoT-MoS₂ layer onto the analyte WE silver ink layer followed by thermal treatment. In some aspects, the method comprises, coating a single-walled carbon nanotube (SWCNT) layer onto the RE Ag/AgCl layer. In some aspects, the method comprises coating a poly (vinyl butyral) (PVB) layer on the Ag/AgCl coated RE and subsequently coating a ion selective membrane (ISM).

In some aspects, the method comprises electrodepositing a gold layer onto the pH WE carbon ink layer. In some aspects, the gold layer is depositing using cyclic voltammetry (CV) for 1 to 50 cycles, 2 to 40 cycles, 3 to 30 cycles, or about 10 to 20 cycles. In some aspects, the method comprises electrodepositing polyaniline (PANI) nanofibers onto the gold layer. In some aspects, prior to deposition, the PANI solution is stirred for 1 hour to 24 hours, 2 hours to 12 hours, 3 hours to 6 hours or about 4 hours. In some aspects, the PANI solution is stirred for about 4 hours, about 12 hours, or about 24 hours. In some aspects, the PANI layer is depositing using cyclic voltammetry (CV) for 1 to 50 cycles, 2 to 40 cycles, 3 to 30 cycles, or about 10 to 20 cycles.

In some aspects, the method comprises depositing a GO/PEDOT:PSS connection layer connecting the first and second temperature sensor coated patterns. In some aspects, the method comprises coating a polydimethylsiloxane (PDMS) layer on the GO/PEDOT:PSS connection layer followed by curing. In some aspects, the method depositing a polyimide film on the PDMS and GO/PEDOT:PSS connection layer.

Provided herein is a method of preparing a multi-parametric water sensor comprising a first analyte sensor and a second analyte sensor. In some aspects, the method comprises cutting into a first surface of a substrate a first analyte sensor working electrode (WE) the counter electrode (CE), and reference electrode (RE) pattern. In some aspects, the method comprises printing carbon ink on the WE and CE patterns. In some aspects, the method comprises printing silver ink on the RE pattern. In some aspects, an upper portion of the RE is printed with Ag/AgCl ink. In some aspects, the method comprises annealing the printed inks. In some aspects, copper is electrodeposited on the WE pattern. In some aspects, the copper layer is depositing using cyclic voltammetry (CV) for 1 to 50 cycles, 2 to 40 cycles, 3 to 30 cycles, or about 10 to 20 cycles. In some aspects, the method comprises coating a poly (vinyl butyral) (PVB) layer on the Ag/AgCl coated RE.

In some aspects, the method comprises cutting into a first surface of a substrate a second analyte sensor working electrode (WE) the counter electrode (CE), and reference electrode (RE) pattern. In some aspects, the method comprises printing carbon ink on the WE and CE patterns. In some aspects, the method comprises printing silver ink on the RE pattern. In some aspects, an upper portion of the RE is printed with Ag/AgCl ink. In some aspects, the method comprises annealing the printed inks. In some aspects, copper is electrodeposited on the WE pattern. In some aspects, the copper layer is depositing using cyclic voltammetry (CV) for 1 to 50 cycles, 2 to 40 cycles, 3 to 30 cycles, or about 10 to 20 cycles. In some aspect, a perfluorinated polymer coating comprising a polytetrafluoroethylene (PTFE) is applied to the electrodeposited copper layer. In some aspects, a polyaniline (PANI) layer is electrodeposited on the perfluorinated polymer coating layer. In some aspects, prior to deposition, the PANI solution is stirred for 1 hour to 24 hours, 2 hours to 12 hours, 3 hours to 6 hours or about 4 hours. In some aspects, the PANI solution is stirred for about 4 hours, about 12 hours, or about 24 hours. In some aspects, the PANI layer is depositing using cyclic voltammetry (CV) for 1 to 50 cycles or 2 to 40 cycles. In some aspects, the method comprises coating a poly (vinyl butyral) (PVB) layer on the Ag/AgCl coated RE.

In some aspects, the method further comprises cutting into a second surface of the substrate a pH sensor working electrode (WE) and reference electrode (RE) pattern. In some aspects, the method comprises printing carbon ink on the WE and printing silver ink on the RE patterns. In some aspects, an upper portion of the RE is printed with Ag/AgCl ink. In some aspects, a polyaniline (PANI) layer is electrodeposited on the WE carbon layer. In some aspects, prior to deposition, the PANI solution is stirred for 1 hour to 24 hours, 2 hours to 12 hours, 3 hours to 6 hours or about 4 hours. In some aspects, the PANI solution is stirred for about 4 hours, about 12 hours, or about 24 hours. In some aspects, the PANI layer is depositing using cyclic voltammetry (CV) for 1 to 50 cycles or 2 to 40 cycles. In some aspects, the method comprises, coating a single-walled carbon nanotube (SWCNT) layer onto the RE Ag/AgCl layer.

In some aspects, the method further comprises cutting into a second surface of the substrate a temperature sensor first connection channel and second connection channel. In some aspects, the method comprises printing silver ink on the first connection channel and the second connection channel. In some aspects, the ink is annealed. In some aspects, a further pattern is prepared connecting the first connection channel and the second connection channel. In some aspects, the method comprises depositing a GO/PEDOT:PSS connection layer on the pattern connecting on the first connection channel and the second connection channel. In some aspects, the method comprises coating a polydimethylsiloxane (PDMS) layer on the GO/PEDOT:PSS connection layer followed by curing. In some aspects, the method depositing a polyimide film on the PDMS and GO/PEDOT:PSS connection layer.

In some aspects, the multi-parametric water sensor comprising an analyte sensor or the multi-parametric water sensor comprising a first analyte sensor and a second analyte sensor is housed in an enclosure. In some aspects, the enclosure comprises a sensor holder, readout circuitry, and a DAS. In some aspects, the enclosure comprises a sensing section that allows water to enter through openings in the top cover, while the data logger remains securely enclosed to protect its circuitry from water exposure. In some aspects, the enclosure further comprises a buffer zone that isolates the sensors from direct water flow, thereby improving measurement accuracy.

### Numbered Aspects

The present application also provides aspects as set forth in the following numbered items including any combinations thereof:

1. A multi-parametric water sensor, comprising:
a substrate;
a sensor array; and
an enclosure comprising a water impermeable material;
the sensor array comprising:
an analyte sensor comprising a working electrode (WE) and a reference electrode (RE) disposed on a first surface of the substrate;
a pH sensor comprising a working electrode (WE) and a reference electrode (RE) disposed on the first surface or a second surface of the substrate; and
a temperature sensor disposed on the first surface or a second surface of the substrate; and
wherein the enclosure comprises a sensor section and a water permeable section.

2. The multi-parametric water sensor of item 1, wherein the analyte is an ion, a dissolved gas, or a combination thereof.

3. The multi-parametric water sensor of any preceding item, wherein the analyte sensor is a potentiometric sensor.

4. The multi-parametric water sensor of any preceding item, wherein the analyte sensor is an ammonia sensor.

5. The multi-parametric water sensor of item 4, wherein the ammonia sensor measures total ammonia, wherein the total ammonia is the sum of ammonium (NH₄⁺) and ammonia (NH₃) in solution.

6. The multi-parametric water sensor of items 1-3, wherein the analyte sensor is a nitrate sensor.

7. The multi-parametric water sensor of item 6, wherein the nitrate sensor comprises a poly(3-octyl-thiophene) (POT)-MoS₂ nanocomposite.

8. The multi-parametric water sensor of item 7, wherein the ratio of POT to MoS₂ is about 1:1 to about 1:6.

9. The multi-parametric water sensor of item 8, wherein the ratio of POT to MoS₂ is about 1:2 to about 1:5.

10. The multi-parametric water sensor of item 9, wherein the ratio of POT to MoS₂ is about 1:3 to about 1:5, or about 1:4 to about 1:5.

11. The multi-parametric water sensor of items 6-10, wherein the nitrate sensor comprises an ion selective membrane (ISM).

12. The multi-parametric water sensor of item 11, wherein the (POT)-MoS₂ nanocomposite is immobilized in the ISM.

13. The multi-parametric water sensor of items 11-12, wherein the ISM is selective for nitrate ions.

14. The multi-parametric water sensor of items 11-13, wherein the ISM is uniform.

15. The multi-parametric water sensor of items 11-14, wherein the nitrate sensor is selective for nitrate over interring ions.

16. The multi-parametric water sensor of items 11-15, wherein the nitrate sensor is selective for nitrate over Cl⁻, SO₄²⁻, HCO₃⁻, PO₄³⁻, and NO₂⁻, or a combination thereof.

17. The multi-parametric water sensor of any preceding item, wherein the RE comprises silver.

18. The multi-parametric water sensor of item 17, wherein the RE comprises Ag/AgCl.

19. The multi-parametric water sensor of item 18, wherein the RE further comprises a single-walled carbon nanotube (SWCNT) layer.

20. The multi-parametric water sensor of item 19, wherein the RE further comprises a poly (vinyl butyral) (PVB) coating.

21. The multi-parametric water sensor of any preceding item, wherein the pH sensor comprises electrodeposited gold.

22. The multi-parametric water sensor of any preceding item, wherein the pH sensor further comprises a thermoplastic resin membrane.

23. The multi-parametric water sensor of item 22, wherein the thermoplastic resin is a polyaniline (PANI) resin.

24. The multi-parametric water sensor of any preceding item, wherein the analyte sensor RE and the pH sensor RE is the same RE electrode, and wherein the RE is in communication with both the analyte sensor and the pH sensor.

25. The multi-parametric water sensor of any preceding item, wherein the temperature sensor comprises a graphene oxide-Poly(3,4-ethylenedioxythiophene):polystyrene sulfonate (GO-PEDOT:PSS) composite.

26. The multi-parametric water sensor of item 25, wherein the temperature sensor further comprises a polydimethylsiloxane (PDMS) layer.

27. The multi-parametric water sensor of item 26, wherein the temperature sensor further comprises a polyimide film.

28. The multi-parametric water sensor of any preceding item, wherein the analyte sensor is configured to resist biofouling.

29. The multi-parametric water sensor of any preceding item, wherein the multi-parametric water sensor comprises two analyte sensors.

30. The multi-parametric water sensor of item 29, wherein the two analyte sensors are disposed on the first surface of the substrate.

31. The multi-parametric water sensor of item 30, wherein the two analyte sensors are configured to resist biofouling.

32. The multi-parametric water sensor of item 29, wherein the pH sensor and the temperature sensor are disposed on the second surface of the substrate.

33. The multi-parametric water sensor of items 29-31, wherein a first analyte sensor comprises a nitrate sensor and a second analyte sensor comprises an ammonia sensor.

34. A multi-parametric water sensor, comprising:
a substrate;
a sensor array; and
an enclosure comprising a water impermeable material;
the sensor array comprising:
a first analyte sensor comprising a working electrode (WE), a counter electrode (CE), and a reference electrode (RE) disposed on a first surface of the substrate;
a second analyte sensor comprising a working electrode (WE), a counter electrode (CE), and a reference electrode (RE) disposed on the first surface of the substrate;
a pH sensor comprising a working electrode (WE) and a reference electrode (RE) disposed on a second surface of the substrate; and
a temperature sensor disposed on the second surface of the substrate; and
wherein the enclosure comprises a sensor section and a water permeable section.

35. The multi-parametric water sensor of item 34, wherein the first analyte sensor is a nitrate sensor.

36. The multi-parametric water sensor of item 35, wherein the nitrate sensor WE and the CE comprise carbon.

37. The multi-parametric water sensor of item 35, wherein the nitrate sensor WE further comprises copper.

38. The multi-parametric water sensor of items 35-37, wherein the nitrate sensor RE comprises silver.

39. The multi-parametric water sensor of item 38, wherein the nitrate sensor RE comprise Ag/AgCl.

40. The multi-parametric water sensor of item 39, wherein the nitrate sensor RE further comprises a poly (vinyl butyral) (PVB) coating.

41. The multi-parametric water sensor of item 35-40, wherein the nitrate sensor CE comprises platinum.

42. The multi-parametric water sensor of item 34-41, wherein the second analyte sensor is an ammonium sensor.

43. The multi-parametric water sensor of item 42, wherein the ammonium sensor WE and the CE comprise carbon.

44. The multi-parametric water sensor of item 43, wherein the ammonium sensor WE further comprises copper.

45. The multi-parametric water sensor of item 44, wherein the ammonium sensor WE further comprises a perfluorinated polymer coating comprising a polytetrafluoroethylene (PTFE).

46. The multi-parametric water sensor of item 45, wherein the ammonium sensor WE further comprises a thermoplastic resin membrane.

47. The multi-parametric water sensor of item 46, wherein the thermoplastic resin is a polyaniline (PANI) resin.

48. The multi-parametric water sensor of items 42-47, wherein the ammonium sensor RE comprise silver.

49. The multi-parametric water sensor of item 48, wherein the ammonium sensor RE comprise Ag/AgCl.

50. The multi-parametric water sensor of item 49, wherein the ammonium sensor RE further comprises a poly (vinyl butyral) (PVB) coating.

51. The multi-parametric water sensor of items 42-50, wherein the ammonium sensor CE comprises platinum.

52. The multi-parametric water sensor of items 34-51, wherein the pH sensor comprises a working electrode (WE) and a reference electrode (RE).

53. The multi-parametric water sensor of item 52, wherein the pH sensor WE and the RE comprise carbon.

54. The multi-parametric water sensor of item 53, wherein the pH sensor WE and the RE further comprise silver.

55. The multi-parametric water sensor of item 54, wherein the pH sensor WE and the RE further comprise Ag/AgCl.

56. The multi-parametric water sensor of items 52-55, wherein the pH sensor WE further comprises a thermoplastic resin membrane.

57. The multi-parametric water sensor of item 56, wherein the thermoplastic resin is a polyaniline (PANI) resin.

58. The multi-parametric water sensor of items 52-57, wherein the pH sensor RE further comprises a single-walled carbon nanotube (SWCNT) layer.

59. The multi-parametric water sensor of item 58, wherein the pH sensor RE further comprises a poly (vinyl butyral) (PVB) coating.

60. The multi-parametric water sensor of item 34-59, wherein the temperature sensor comprises silver.

61. The multi-parametric water sensor of item 60, wherein the temperature sensor comprises a graphene oxide-Poly(3,4-ethylenedioxythiophene):polystyrene sulfonate (GO-PEDOT:PSS) composite.

62. The multi-parametric water sensor of item 61, wherein the temperature sensor further comprises a polydimethylsiloxane (PDMS) layer.

63. The multi-parametric water sensor of item 62, wherein the temperature sensor further comprises a polyimide film.

64. The multi-parametric water sensor of any preceding item, wherein the enclosure further comprises a buffer zone between the sensor section and the water permeable section.

65. The multi-parametric water sensor of item 34-64, wherein the first analyte sensor and the second analyte sensor are configured to resist biofouling.

66. The multi-parametric water sensor of any preceding item, wherein the substrate is a flexible substrate.

67. The multi-parametric water sensor of any preceding item, wherein the substrate is a thermoplastic and/or thermosetting film.

68. The multi-parametric water sensor of any preceding item, wherein the substrate is a polyimide film, a perfluorinated sulfonic-acid isomer film, sulfonated tetrafluoroethylene fluoropolymer-copolymer film, or a polyethylene terephthalate (PET) film.

69. The multi-parametric water sensor of item 68, wherein the substrate is a polyethylene terephthalate (PET) film.

70. The multi-parametric water sensor of any preceding item, wherein the substrate has a thickness of 50 μm to 500 μm, or 75 μm to 400 μm, or 100 μm to 350 μm, or 110 μm to 250 μm, or 115 μm to 200 μm.

71. The multi-parametric water sensor of any preceding item, further comprising a data acquisition system (DAS), wherein the DAS comprises a processor; a communication unit; a memory unit; and a power supply unit, and wherein the data acquisition system is in communication with the sensor array.

72. The multi-parametric water sensor of item 71, wherein the memory unit is communicatively coupled to the processor, the memory unit having stored thereon computer software comprising a set of instructions that, when executed by the processor, causes the data acquisition unit to receive sensor data from the sensor array; and send, via the communication unit, the sensor data to an external device.

73. The multi-parametric water sensor of items 71-72, wherein the memory unit comprises a non-transitory computer-readable medium.

74. The multi-parametric water sensor of item 73, wherein the sensor data are sent, via the communication unit, to an Internet of Things (IoT) cloud server configured to interact with one or more IoT-capable devices.

75. The multi-parametric water sensor of items 71-74, wherein the DAS comprises a microcontroller unit.

76. The multi-parametric water sensor of any preceding item, wherein the enclosure further comprises a DAS section, wherein the DAS section separates the DAS from the water.

77. The multi-parametric water sensor of item 72, wherein the instructions comprise a signal calibration of at least one electrode.

78. The multi-parametric water sensor of item 77, wherein the instructions comprise a pH-based signal correction, a temperature-based signal correction, or a combination thereof.

79. The multi-parametric water sensor of any one of items 1-28, wherein the analyte sensor has an analyte detection range of about 0.7 ppm or more, about 0.8 ppm or more, about 0.7 ppm to about 100 ppm, or about 0.8 to about 100 ppm.

80. The multi-parametric water sensor of item 33 or 35, wherein the nitrate sensor has an analyte detection range of about 0.8 ppm to about 100 ppm.

81. The multi-parametric water sensor of item 33 or 42, wherein the ammonium sensor has an analyte detection range of about 0.7 ppm to about 10 ppm.

82. The multi-parametric water sensor of any one of items 1-28, wherein the analyte sensor has a coefficient of variance between measurements of not more than about 10%, not more than about 8%, not more than about 7%, not more than about 6%, not more than about 5%, not more than about 4%, not more than about 2%, or not more than about 1%.

83. The multi-parametric water sensor of any one of item 34, wherein the first analyte sensor, the second analyte sensor, or both has a coefficient of variance between measurements of not more than about 10%, not more than about 8%, not more than about 7%, not more than about 6%, not more than about 5%, not more than about 4%, not more than about 2%, or not more than about 1%.

84. The multi-parametric water sensor of any one of items 1-28, wherein the analyte sensor has a coefficient of variance between measurements of not more than about 10%, about 9%, about 8%, or about 5% before and after a dynamic folding test, wherein in the dynamic folding test the flexible plant sensor in an unbent orientation is bent to a 90° angle, returned to the unbent orientation, and repeated up to 100 cycles, 500 or 1000 cycles.

85. The multi-parametric water sensor of any one of item 34, wherein the first analyte sensor, the second analyte sensor, or both has a coefficient of variance between measurements of not more than about 10%, about 9%, about 8%, or about 5% before and after a dynamic folding test, wherein in the dynamic folding test the flexible plant sensor in an unbent orientation is bent to a 90° angle, returned to the unbent orientation, and repeated up to 100 cycles, 500 or 1000 cycles.

86. A method of measuring one or more analytes in an aqueous environment, comprising:
placing the multi-parametric water sensor according to any one of items 1-85 in the aqueous environment;
measuring a signal corresponding to one or more analytes in the aqueous environment;
measuring at least one environmental parameter comprising:
i) measuring a signal corresponding to a pH of the aqueous environment;
ii) measuring a signal corresponding to a temperature of the aqueous environment; or
iii) both i) and ii); and
obtaining as a data output from the multi-parametric water sensor an amount of the one or more analytes in the aqueous environment, wherein the amount of at least one of the analytes is based on the measured value of the at least one environmental parameter of the aqueous environment.

87. The method of item 86, further comprising applying an electrochemical potential across one or more sensors of the sensor array to prevent or reduce biofouling on the one or more sensors.

88. The method of any one of items 86-87, wherein the environmental parameter comprises measuring a signal corresponding to a pH of the aqueous environment and measuring a signal corresponding to a temperature of the aqueous environment.

89. The method of any one of items 86-87, wherein the method comprises measuring total ammonia in the aqueous environment.

90. The method of item 89, wherein the method further comprises measuring the signal corresponding to the pH of the aqueous environment, measuring the signal corresponding to the temperature of the aqueous environment, and determining the ratio of ammonium (NH₄⁺) to ammonia (NH₃) in the aqueous environment.

91. The method of any one of items 86-90, wherein the method further comprises measuring a signal corresponding to an amount of nitrate in the aqueous environment.

92. The method of any one of items 86-91, wherein each of the measuring steps comprises continuous measurement.

93. The method of any one of items 86-91, wherein each of the measuring steps comprises measurement every 1 minute to 10 minutes or every 2 minutes to 3 minutes.

94. The method of any one of items 86-93, wherein the multi-parametric water sensor processes the sensor data from the one or more sensors to produce the data output.

95. The method of item 94, wherein the processed data are stored in the memory unit of the multi-parametric water sensor.

96. The method of any one of items 94-95, wherein the processed data are sent, via the communication unit, to the IoT cloud server configured to interact with the one or more IoT-capable devices.

97. The method of any one of items 86-96, wherein one or more of the measuring steps, the data output, or both is conducted automatically by the multi-parametric water sensor without user intervention.

98. The method of item 97, wherein each of the measuring steps and the data output is conducted automatically by the multi-parametric water sensor without user intervention after a step of activating the multi-parametric water sensor.

99. The method of item 86, wherein the data output is obtained from the multi-parametric water sensor.

100. The method of item 86, wherein the data output is obtained from the IoT capable device.

101. The method of any one of items 86-100, wherein the aqueous environment comprises an environment selected from an aquaculture, a hydroponic system, an aquaponic system, watersheds, streams, ponds, lakes, rivers, water reservoirs, fisheries, or wastewater systems.

102. A method of determining a presence or an amount of ammonia (NH₃) in an aqueous environment based on the amount of total ammonia using the multi-parametric water sensor according to any one of items 1-85.

103. The method of item 102, further comprising determining a presence or an amount of nitrate in the aqueous environment.

104. The method of any one of items 102-103, wherein the aqueous environment comprises an environment selected from an aquaculture, a hydroponic system, an aquaponic system, watersheds, streams, ponds, lakes, rivers, water reservoirs, fisheries, or wastewater systems.

105. The method of any one of items 102-104, further comprising reducing or increasing agricultural fertilizer based on the presence or the amount of ammonia (NH₃) in the aqueous environment.

106. The method of item 105, further comprising reducing or increasing agricultural fertilizer based on the presence or the amount of nitrate in the aqueous environment.

107. A method for preparing a multi-parametric water sensor, comprising
preparing a sensor array, comprising:
disposing a pH sensor working electrode (WE) pattern and an analyte WE pattern on a substrate;
disposing a reference electrode (RE) patten on the substrate between the analyte WE pattern and the pH sensor WE pattern;
disposing a temperature sensor WE pattern comprising a first pattern and second pattern on the substrate, wherein the analyte sensor WE, the RE, and the pH WE are disposed between the first pattern and the second pattern;
printing an ink comprising silver on the analyte sensor WE pattern and the temperature sensor WE pattern;
printing an ink comprising carbon on the pH sensor pattern;
printing an ink comprising Ag/AgCl on the RE pattern;
electrodepositing a gold layer onto the pH WE carbon ink layer;
depositing a GO/PEDOT:PSS connection layer connecting the first and second temperature sensor coated patterns; and
coating a PoT-MoS₂ layer onto the analyte WE silver ink layer.

108. The method of item 107, wherein the method comprises a sintering step after the steps of printing the ink comprising silver on the analyte sensor WE pattern and the temperature sensor WE pattern; printing the ink comprising carbon on the pH sensor pattern; and printing the ink comprising Ag/AgCl on the RE pattern.

109. The method of items 107-108, comprising thermally treating the PoT-MoS₂ layer coated onto the analyte WE silver ink layer.

110. The method of items 107-109, comprising coating a single-walled carbon nanotube (SWCNT) layer onto the RE Ag/AgCl layer.

111. The method of item 110, comprising coating a membrane on the SWCNT/Ag/AgCl RE, the membrane comprising a poly (vinyl butyral) (PVB) layer and an ion selective membrane (ISM).

112. The method of item 111, wherein the gold is deposited on the pH WE carbon ink layer using cyclic voltammetry (CV) for 1 to 50 cycles, 2 to 40 cycles, 3 to 30 cycles, or about 10 to 20 cycles.

113. The method of item 112, further comprising electrodepositing polyaniline (PANI) nanofibers onto the gold layer.

114. The method of item 113, wherein the method further comprises, prior to depositing the PANI, stirring the PANI solution for 1 hour to 24 hours, 2 hours to 12 hours, 3 hours to 6 hours or about 4 hours.

115. The method of items 113-114, wherein the PANI layer is depositing using cyclic voltammetry (CV) for 1 to 50 cycles, 2 to 40 cycles, 3 to 30 cycles, or about 10 to 20 cycles.

116. The method of items 107-115, comprising depositing a GO/PEDOT:PSS connection layer connecting the first and second temperature sensor coated patterns.

117. The method of item 116, further comprising coating a polydimethylsiloxane (PDMS) layer on the GO/PEDOT:PSS connection layer followed by curing.

118. The method of item 117, further comprising depositing a polyimide film on the PDMS and GO/PEDOT:PSS connection layer.

119. A method for preparing a multi-parametric water sensor, comprising
preparing a sensor array, comprising:
disposing on a first surface of a substrate a first analyte sensor working electrode (WE) pattern, a counter electrode (CE) pattern, and a reference electrode (RE) pattern;
disposing on the first surface of the substrate a second analyte sensor WE pattern, a CE pattern, and a RE pattern;
disposing on a second surface of the substrate a pH sensor WE pattern and RE pattern;
disposing on the second surface of the substrate a temperature sensor first connection channel pattern and second connection channel pattern;
printing an ink comprising carbon on the first analyte sensor WE and CE patterns, the second analyte sensor WE and CE patterns, and the pH sensor WE pattern;
printing an ink comprising silver on the first analyte sensor, the second analyte sensor, pH sensor RE patterns, and the first connection channel pattern and the second connection channel pattern of the temperature sensor;
printing an ink comprising Ag/AgCl on a portion of the first analyte sensor and a portion of the second analyte sensor RE patterns;
electrodepositing copper on the first analyte sensor and the second analyte sensor WE pattern;
electrodepositing on the second analyte sensor copper layer a perfluorinated polymer coating comprising a polytetrafluoroethylene (PTFE);
electrodepositing on the second analyte sensor copper and perfluorinated polymer coating layers a polyaniline (PANI) layer;
electrodepositing on the pH sensor WE carbon layer a polyaniline (PANI) layer; and
depositing a GO/PEDOT:PSS connection layer connecting the first connection channel pattern and the second connection channel pattern of the temperature sensor.

120. The method of item 119, wherein the method comprises a sintering step after the steps of printing the ink comprising carbon on the first analyte sensor WE and CE patterns, the second analyte sensor WE and CE patterns, and the pH sensor WE pattern; printing the ink comprising silver on the first analyte sensor, the second analyte sensor, pH sensor RE patterns, and the first connection channel pattern and the second connection channel pattern of the temperature sensor; and printing the ink comprising Ag/AgCl on the portion of the first analyte sensor and the portion of the second analyte sensor RE patterns.

121. The method of items 119-120, wherein the copper layer is depositing using cyclic voltammetry (CV) for 1 to 50 cycles, 2 to 40 cycles, 3 to 30 cycles, or about 10 to 20 cycles.

122. The method of item 119, further comprising coating a poly (vinyl butyral) (PVB) layer on the first analyte sensor RE and the second analyte sensor RE.

123. The method of item 119, wherein the method further comprises, prior to depositing the PANI, stirring the PANI solution for 1 hour to 24 hours, 2 hours to 12 hours, 3 hours to 6 hours or about 4 hours.

124. The method of items 119 or 1123, wherein the PANI layer is depositing using cyclic voltammetry (CV) for 1 to 50 cycles or 2 to 40 cycles.

125. The method of items 119, comprising coating a single-walled carbon nanotube (SWCNT) layer onto the pH sensor RE Ag/AgCl layer.

126. The method of item 119, further comprising coating a polydimethylsiloxane (PDMS) layer on the GO/PEDOT:PSS connection layer followed by curing.

127. The method of item 119, further comprising depositing a polyimide film on the PDMS and GO/PEDOT:PSS connection layer.

128. The method of any one of items 107-127, further comprising disposing the sensor array in an enclosure.

### EXAMPLES

The following examples are intended to exemplify the present disclosures and are not limitations of the claimed invention. All molecules, compositions, methods, assays, and results disclosed in the examples form part of the present invention.

### Example 1—Multi-Parametric Water Sensor with Nitrate Sensor: Fabrication of Nitrate Sensor

A multi-parametric water sensor with nitrate, pH, and temperature sensors was fabricated on a 125 μm thick polyethylene terephthalate (PET) substrate. A schematic illustration of the integrated sensor array is shown in FIG. 1A. The sensor was housed within a waterproof, three-dimensional (3D) printed box that contains the readout circuitry, data communication module, and the sensor array. The sensing section is exposed to incoming water through holes in the top cover, while the data logger is securely enclosed inside the box to protect the circuitry from water ingress. The system-level flow diagram illustrates the processes of data acquisition, analysis, and communication between the circuit and the server to enable multiplexed sensing.

FIG. 2 shows the step-by-step fabrication process of the sensor array. The potentiometric nitrate sensor consists of a poly(3-octyl-thiophene) (POT)-MoS₂ nanocomposite immobilized with an ion-selective membrane (ISM), while the pH sensor is made from polyaniline (PANI). The PoT-MoS₂ functions as the solid contact ion-to-electron transducing layer, while the ISM layer provides selectivity to nitrate ions. PANI-based electrodes are highly sensitive to H₃O⁺ ions. The redox equilibrium between the H₃O⁺ and PANI phase transitions is ideal for pH sensing. Furthermore, PANI provides high surface area, potential stability, biocompatibility, and reproducible performance of the pH sensor. A poly (vinyl butyral) (PVB)-coated Ag/AgCl electrode acts as a shared RE for both the nitrate and pH sensors. A PVB polymeric membrane provides long-term stability and prevents chloride leaching. These potentiometric sensors generate potential differences (ΔE) proportional to the concentration of the respective ions between the working electrode and the reference electrode.

The cyclic voltammograms obtained for the PoT-MoS₂ and PoT-MoS₂/ISM-based electrodes displayed distinct reversible oxidation and reduction reactions in a 0.5 mM ferri/ferrocyanide redox probe solution, as shown in FIG. 3A. Notably, the oxidation current recorded for the PoT-MoS2-based electrode (1.13 mA) was higher than that of the PoT-MoS2/ISM-based electrode (0.227 mA). After the addition of the ISM, the PoT-MoS2-based electrode exhibited diminished oxidation and reduction peaks, likely due to slower ion exchange or the ISM's high selectivity, which may have hindered the uptake of ferri/ferrocyanide ions (inset of FIG. 3A). Nonetheless, the electrode demonstrated significant redox capacitance and electroactivity, enabling selective uptake or release of hydrophilic NO₃⁻ ions. The weight ratio of POT to MoS2 in the composite was evaluated with CV measurements that were conducted on composites with different weight ratios. FIG. 3B shows that as the weight ratio of POT to MoS2 changes from 1:1 to 1:6, the ΔE value increases at lower weight ratios, peaks at ΔE=371 mV at a 1:4 ratio, and then decreases at higher weight ratios. Hence, POT to MoS2 weight ratio of 1:4 was used for all subsequent experiments.

Scanning electron microscopic (SEM) images provide insight into the arrangement and structure of the ion-to-electron transducing layers on the Ag surface. The Ag film displays a uniform distribution of crystalline-sized Ag micro-texture, as seen in FIG. 4A. The MoS2 sheets are integrated with POT due to electrostatic interactions between the two materials (FIG. 4B).

FIG. 1B depicts the open circuit potentials of the NO₃⁻ sensor in deionized water with NO₃⁻ concentrations ranging from 1 to 1000 ppm. Three identical sensors (F1, F2, and F3) demonstrate similar behavior during 90 minutes of continuous operation. The potential changes that occurred when changing the solutions are not detailed in the plot. The sensor achieved a stable potential value in under 60 seconds after a concentration change. FIG. 1C presents the calibration curve, illustrating the potential difference across various nitrate concentrations. The sensors exhibit a linear, near-Nernstian behavior (with a theoretical sensitivity of 59 mV per decade for ISEs based on monovalent ions) and a sensitivity of −50 mV per decade of concentration, accompanied by a relatively low standard deviation. The strong electroactivity and redox properties of the PoT-MoS2 layer may enhance sensitivity for nitrate detection. Additionally, the high hydrophobicity of the layer minimizes water accumulation between the ISM and the Ag current collector, further improving sensor sensitivity. The limit of detection (LOD) was calculated to be 0.44 ppm based on the calibration curve obtained.

### Example 2—Multi-Parametric Water Sensor with Nitrate Sensor: Device Fabrication

All the sensors were fabricated on a 125 μm thick polyethylene terephthalate (PET) substrate, as shown in FIG. 2A. First, the electrode patterns were designed in SolidWorks, and then the patterns were cut onto the PET sheet using a programmable cutter (p28, USCutter, USA). The working electrodes for the nitrate and pH sensors (WENitrate, WEpH), composed of 3 mm diameter circles connected to 1.25 mm wide traces, were screen-printed using Ag ink for nitrate and carbon ink for pH. A common reference electrode (RE), with dimensions similar to WENitrate and WEpH, was screen-printed with Ag ink. Additionally, two 2 mm wide electrode patterns for the temperature sensor (T) were placed near the outer edges of the PET substrate, covering WENitrate, WEpH, and RE. The temperature sensor's electrodes were screen-printed with Ag ink. A 12 mm arc connected the two 2 mm wide traces. During the screen-printing process, a shadow mask was used to cover the arc. Following this, the circular area of the RE was screen printed with Ag/AgCl ink. Finally, all electrodes were sintered at 120° C. for 15 minutes, realizing the structure depicted in FIG. 2B.

The nitrate sensor's working electrode (WENitrate) was modified with PoT-MoS2 and ISM. The PoT-MoS2 nanocomposite was prepared by dissolving 13 mg of POT and 52 mg of MoS2 in 5 ml of THF to get a 1:4 weight ratio of POT to MoS₂. The weight ratio of POT to MoS2 was varied to study the influence of material composition on the redox properties of different PoT-MoS2 nanocomposites. The solution was sonicated for 30 minutes using a horn sonicator (Q500, QSonica) at 40% power to obtain a homogeneous solution. A high-precision automated spray coater (Nordson E2 EFD, UK) was used to apply a uniform layer of PoT-MoS2 onto the Ag-printed WENitrate. In this process, the PoT-MoS2 solution was sprayed from a 10-cc syringe under 20 psi air pressure, followed by thermal treatment on a hot plate at 65° C. for 1 h.

Both the pH and nitrate sensors shared the same reference electrode. To mitigate the diffusion of the chloride ions, a single-walled carbon nanotube (SWCNT) layer was incorporated between the reference electrode (RE) membrane and the Ag/AgCl layer to act as a surface for the adsorption and retention of chloride ions. SWCNT dispersion was prepared by mixing 20 mg of SWCNT with 50 mg of SDS dissolved in 20 mL of DI water and stirring for 15 minutes at 600 rpm. Next, the mixture was horn sonicated for 1 hour at 50% power. The dispersion was centrifuged at 13500 RPM for 1 hour to remove the unexfoliated nanotubes from the supernatant. The obtained solution was spray-coated on top of the Ag/AgCl layer of the RE before applying the reference membrane, as shown in FIG. 2C.

Following this, gold (Au) was electrodeposited onto the WpH surface, as shown in FIG. 2D. The deposition was carried out using a solution of 0.5 M H2SO4 containing 5 mM HauCl4. A three-electrode system was used, with a commercial glass reference electrode (MF-2052, Basi) and a platinum counter electrode (MW-1033, Basi), against the printed carbon electrode. Cyclic voltammetry (CV) was performed for 20 cycles at a scan rate of 0.05 V/s within a potential range of −2 V to −0.5 V. Subsequently, polyaniline (PANI) nanofibers were deposited onto the Au-deposited electrode using the electro-polymerization method. The electrodeposition process involved immersing the electrodes in a solution of 0.1 M aniline in 1 M H₂SO₄ and conducting 20 cycles of CV, with a potential range from −0.4 V to 1 V at a scan rate of 50 mV/s. The device structure after Au and PANI electrodeposition is shown in FIG. 2E.

The RE membrane was prepared by dissolving 1.58 g of poly (vinyl butyral) (PVB) and 1.00 g of NaCl in 20 mL of methanol. This mixture was sonicated for 30 min in an ice bath. The ISM solution was prepared by mixing 12.5 mg methyl triphenyl phosphonium bromide, 96.5 mg nitrocellulose, 1.73 ml 2-nitrophenyl octyl ether, 470 mg polyvinyl chloride, and 125 mg tri dodecyl methyl ammonium nitrate in 4.73 ml THF. Finally, as shown in FIG. 2F, 20 μL of ISM solution was drop cast on the WENitrate. Following that, 30 μL of the RE membrane solution was drop cast on the SWCNT-coated RE. The sensor was left to dry for 24 hours.

The arc-shaped temperature sensor was made from a graphene oxide-Poly(3,4-ethylenedioxythiophene):polystyrene sulfonate (GO-PEDOT:PSS) composite. A 1 mg/ml concentration of GO powder was dispersed in deionized (DI) water and stirred magnetically for 20 minutes at 500 rpm. It was then subjected to mild bath sonication for another 20 minutes to achieve a homogenous solution. The GO dispersion was mixed with PEDOT:PSS in a 1:1 ratio under continuous stirring at 1000 rpm for 30 minutes. The GO/PEDOT:PSS layer was formed through spray deposition for 30 cycles at 35° C., as shown in FIG. 1G. The SEM image of the GO/PEDOT:PSS layer is shown in FIG. 4I. A polydimethylsiloxane (PDMS) layer was selectively spin-coated on top of the temperature sensor using a shadow mask and cured for 60 minutes at 80° C. to protect the temperature-sensitive GO/PEDOT:PSS coating from environmental influences. Finally, the PDMS coating was shielded with a 50 μm thick Kapton tape to further protect the GO/PEDOT:PSS coating. The final structure is shown in FIG. 2H. The sensor was allowed to dry for 24 hours before being immersed in a 3M KCL solution to condition the polymeric membranes.

### Example 3—Multi-Parametric Water Sensor with Nitrate Sensor: Evaluation of Reference Electrode

The performance of the sensors was tested in aquaculture settings, where temperature and pH frequently fluctuate. Water samples were collected from the catfish RAS at the United States Department of Agriculture's Agricultural Research Service (USDA-ARS) Warmwater Aquaculture Research Unit in Stoneville, MS, USA. These water samples were used for all subsequent analyses. FIG. 5A illustrates the calibration curves of the nitrate sensor across temperatures ranging from 10-40° C. The calibration curve shifts upward as the temperature increases. The temperature range was set from 10-40° C. to simulate the seasonal conditions of natural water bodies and RAS systems. Temperature affects the physiological processes of the reared species, such as respiration rate, efficiency of feeding, growth, behavior, reproduction and depuration similarly, FIG. 5B depicts the calibration curve of the nitrate sensor across varying PH levels, with the shift in the calibration curve corresponding to changes in pH levels. As the PH levels of the tested water sample increase, the calibration curve shifts upward. The pH level was adjusted from 6 to 10 to cover the pH variations found in aquaculture ponds and RAS systems. Ensuring repeatability is essential for a high-performance nitrate sensor, especially in environments where nitrate concentrations fluctuate over time. To assess repeatability, the open circuit potential responses of the nitrate sensor were measured over three consecutive cycles, where nitrate concentrations were first varied from 1 to 1000 ppm and then back to 1 ppm in each cycle. The sensor was exposed to each concentration for five minutes, as shown in FIG. 5C. The sensor's dynamic response was tested twice, alternating between high and low NO₃⁻ concentrations. The sensor responded quickly to changes in NO₃⁻ levels, within approximately 30 seconds to reach stability. Furthermore, the sensor exhibited a relative standard deviation (RSD) of 2.10% when responding to the same nitrate concentration after the three cycles of exposure. This demonstrates that the sensor consistently provides reproducible and reliable NO₃⁻ detection over repeated tests.

To evaluate the sensor's selectivity, common interfering ions (Cl⁻, SO₄²⁻, HCO₃⁻, PO₄³⁻, and NO₂⁻) were tested both in the absence and presence of nitrate at a constant concentration of 100 ppm. FIG. 5D shows that the sensor response remained largely unaffected by these interferents. The formation of a uniform ISM layer is crucial, as it should selectively permit the target ion while blocking interfering ions. This uniformity is evident in the SEM image of the ISM layer in FIG. 4C. Moreover, FIGS. 4D-4E display the uniform distribution of the PoT/MoS₂ layer, while the mass percentages of Mo and S are verified from the Energy Dispersive Spectroscopic (EDS) analysis FIG. 4F.

The stability of the fabricated Ag/AgCl RE, with and without a reference membrane coating, was evaluated against a leakless glass Ag/AgCl RE with a 3 M KCl internal electrolyte, serving as electrochemical references whose potential should remain constant in varying ionic environments. The specific composition of the printed RE influences its E0 value in the Nernst equation. However, since E0 remains stable, any deviation can be easily calibrated. The potential over time was monitored during nitrate concentration changes, comparing the performance of the printed Ag/AgCl RE both without any membrane and with a PVB membrane containing NaCl and NaNO3. FIG. 5E shows that the RE coated with the NaCl+NaNO₃ membrane has a slope of −5 mV/dec, which is five-fold less compared to the bare Ag/AgCl RE's slope of −25 m V/dec. This indicates the reduced sensitivity of the RE coated with the NaCl+NaNO₃ membrane to nitrate concentration changes.

FIG. 5F illustrates the stability of the modified Ag/AgCl RE compared to a commercial RE, with KCl concentrations ranging from 0.1-3 M. Without the reference membrane coating, significant fluctuations in the open circuit potential (OCP) were observed due to electrochemical reactions in the AgCl layer, possibly releasing Cl⁻ ions and causing instability. However, the modified Ag/AgCl RE with the reference membrane exhibited minimal OCP changes even with variations in the KCl concentration. The addition of a CNT layer and a PVB-NaCl membrane on the Ag/AgCl RE surface altered the surface area and composition, with the PVB-NaCl membrane acting as a protective barrier, preventing Cl⁻ ion leaching and blocking external Cl⁻ ions.

### Example 4—Multi-Parametric Water Sensor with Nitrate Sensor:Evaluation of pH and Temperature Sensor

The open circuit potential (OCP) measured between the PANI working electrode (WEpH) and the Ag/AgCl RE revealed the electrochemical characteristics of the pH sensor. The PANI-based working electrode, selective for H₃O⁺, and the RE generated potential differences based on the pH changes in the solution. The electrodeposition of a conductive Au layer facilitated the formation of a uniform PANI layer. FIG. 3C depicts the formation of the Au layer through CV conducted over 20 cycles, within a potential range of −2.0 V to −0.5 V at a scan rate of 50 m V/s. The SEM image in FIG. 4G depicts the distribution of the electrodeposited Au over the WEPH. Subsequently, the PANI layer was electro-polymerized on the Au-coated WEpH. Electropolymerization allowed precise control of the coating uniformity on the electrode surface. The oxidative polymerization of aniline began at 0.1 V in the first cycle, forming the initial PANI layer. In subsequent cycles, the polymer film continued to grow, indicated by an increase in redox current, which reflects the conductivity, electroactivity, and uniformity of the film. The SEM image in FIG. 4H shows the continuous PANI layer, and the CV plots in FIG. 3D illustrate the aniline polymerization process. Voltage signals from the pH sensor were recorded by adjusting pH levels between 4 and 10 in both standard buffer solutions and RAS samples collected from USDA-ARS's Warmwater Aquaculture Research Unit in Stoneville, MS, USA. The pH levels of the solutions were adjusted by adding either HCl or NaOH, and the pH values were validated using a commercial pH meter (Mettler Toledo, Columbus, OH). As the solution pH changed, the sensor signals fluctuated initially, but eventually stabilized in approximately 30 seconds. Signals were recorded after the stable values were reached, with measurements lasting for 10 minutes at each concentration. FIG. 6A displays the OCP of the pH sensors in both buffer solutions and USDA water samples (denoted as USWS). The calibration curve for the pH sensor, shown in FIG. 6B, indicates a near-Nernstian linear response with a sensitivity of −57.254 m V/dec in the buffer solution and −56.959 m V/dec in the USDA water sample. This indicates that the pH sensor's response remains highly consistent regardless of the solution medium. Ensuring repeatability is critical for the long-term performance of pH sensors, particularly in environments like RAS and ponds where pH levels fluctuate over time. The pH sensor's repeatability was evaluated by repeatedly exposing it to increasing and decreasing pH levels in USDA water samples. FIG. 6C shows the voltage responses of the pH sensor during cyclic exposure to pH levels of 6, 7, 8, 9, 10, and 11. The sensor exhibited an RSD of less than 3.5% for the same pH level after three consecutive exposures. Additionally, the pH sensor's response was tested under varying solution temperatures, where the temperature ranged from 20° C. to 40° C., as depicted in FIG. 6D. This temperature range was selected to simulate the seasonal conditions of natural water bodies and RAS systems. It was observed that at higher pH levels (i.e., pH 11), the sensor response showed a maximum variation of 39.49 mV with a 20° C. temperature change. An onboard temperature sensor can be provided to compensate for the variations in the pH sensor's response due to temperature fluctuations.

Temperature measurements can reduce or eliminate the effect of temperature fluctuations on the chemical sensor readings. FIG. 6E presents the printed Graphene Oxide/Poly(3,4-ethylenedioxythiophene) polystyrene sulfonate (GO/PEDOT:PSS)-based temperature sensor's response to temperature variations from 20° C. to 40° C., and FIG. 6F shows the calibration plot of the temperature sensor. The sensor was calibrated by first placing it on a hot plate set to room temperature to achieve a stable response. Next, the temperature of the hot plate was gradually increased from 20° C. to 40° C. (referred to as the heating phase). The sensor was immersed in the USDA water sample during this calibration process. As the temperature rises, more charge carriers become available, leading to a reduction in the resistance, indicating the negative temperature coefficient of resistance (TCR) characteristics of PEDOT:PSS [5][6]. The printed GO/PEDOT:PSS-based temperature sensor demonstrates an average sensitivity of 8.13 kΩ/° C. (FIG. 6F). The change in resistance with temperature was found to be linear, with a Pearson's correlation coefficient (R2 value) of 0.951.

### Example 5—Multi-Parametric Water Sensor with Nitrate Sensor: Real-Time Nitrate Monitoring

The multiparametric sensor, which includes nitrate, pH, and temperature sensors along with a custom-designed data logger, was housed in am enclosure (a 3D-printed water-resistant box) and deployed in six RAS tanks (size to be specified) containing healthy catfish. These tanks received regular supplies of fresh water, air flow, and controlled doses of ammonium to promote the growth of beneficial nitrifying bacteria and mitigate ammonia toxicity, vital for maintaining a healthy ecosystem within the RAS. FIG. 7A illustrates a typical catfish tank with a partially submerged sensor box. The active regions of the sensors (i.e., circular areas of the nitrate and pH sensor working electrodes and semicircular arc of the temperature sensor) were exposed to water through holes in the box, while the electrode traces and the data logger were enclosed within the box. FIG. 7B displays the complete system collecting data from the sensor array and transmitting data to the PC. Water from the fish tanks was tested for nitrate concentration, pH, and temperature using commercial test kits: a DR 3900 spectrophotometer with a TNT nitrate kit from Hach, a pH meter from Fisher Scientific, and an HQ 40D handheld multi-parameter meter. Meanwhile, the sensor array continuously recorded nitrate, pH, and temperature levels, which were compared to the results obtained from the commercial kits. In the RAS tanks, pH levels were maintained between 8.09 and 8.66, while the temperature varied from 22.4° to 24.2° C.

FIGS. 7C-7H demonstrate the performance of the developed nitrate and pH sensors in comparison to the commercial kits. In FIG. 7C, the hourly fluctuations in nitrate concentrations recorded by the fabricated and commercial sensors over a 48-hour period in Tanks T1, T3, and T4 are presented. The fabricated sensor arrays, which were deployed in the tanks for 48 hours, provided continuous data, while the kit values were collected at specific intervals. Throughout this duration, the sensor arrays experienced controlled ammonium dosing, air flow, freshwater flow, fish movement, and tank debris. Despite these variables, FIG. 7C illustrates that the nitrate concentrations measured with the sensor closely correlated with the values from the commercial kit. A similar agreement was observed in Tanks T6, T7, and T9, which were subjected to the same conditions for 24 hours, as shown in FIG. 7D. Additionally, the sensor array measured the PH levels in the fish tanks, with the results plotted against those from the commercial pH meter, as depicted in FIGS. 7E-7F. The pH measurements from the sensors closely matched the values obtained from the commercial kits. Nonetheless, a maximum percentage error of 15% was observed between the measurements from the nitrate sensor and the kit, which may be due to the influence of pH level fluctuations on the recorded nitrate levels. To address these variations in nitrate levels, corrections were made based on the corresponding pH values, as detailed below.

Given that the nitrate sensor's response can vary with changes in pH, pH correction is needed to get precise nitrate readings. A multiple linear regression equation is derived from the calibration plots of the nitrate sensor across various pH levels, ranging from pH 6 to pH 10. FIG. 8 outlines the nitrate sensor's responses for varying nitrate and pH levels. The following regression equation was used to apply the corrections with the data logger:

$${NO}_{3}^{-} = {K}_{0} + {K}_{1} \times pH + {K}_{2} \times Voltage$$

The coefficients generated from the MLR resulted as K₀=−88.58, K₁=5.44980, K₂=−0.10751 which were used to adjust the nitrate sensor response. The adjusted values were plotted against the kit values in a scatter plot that shows significant improvement in the R² and root mean square error (RMSE).

Bar plots comparing kit values and sensor measurements for nitrate indicated that the corrected nitrate values aligned more closely with the kit values, resulting in reduced discrepancies. The sensor data from all fish tanks closely matched the kit data, indicating that the correction significantly enhanced the measurement accuracy. FIG. 7G illustrates corrected nitrate levels in fish tanks T1, T3, and T4 over 48 hours at 6 time points, while FIG. 7H shows the values for tanks T6, T7, and T9 over 24 hours at 6 time points.

To quantify the error between the sensor and kit measurements, the nitrate and pH values recorded by the sensors were plotted against the corresponding values measured by the commercial kits, as shown in FIGS. 9A-9C. FIG. 9A shows that the RMSE between the sensor and the commercial kit is 4.779 ppm before compensating for pH variations. FIG. 5B shows an RMSE of 0.072 for the pH sensor, demonstrating the excellent performance of the pH sensor. The correlation between nitrate sensor measurements and kit values was relatively low before pH corrections, attributed to pH variations in the fish tanks. Following the pH correction, a significant improvement in R² and RMSE values was observed in FIG. 5C, where data points are seen closer to the linear fit.

### Example 6—Multi-Parametric Water Sensor with Nitrate Sensor: Bending Test

A bending test was conducted for the multi-parametric sensor by bending the sensor for 100, 500, 1000 cycles and testing sensor response. The results are shown in FIGS. 10A-10J. Voltages in the bending test after 100, 500, 1000 cycles for the nitrate sensor tested continuously after bending at each concentration for 5 minutes, with increasing number of cycles the OCP increases (FIG. 10A). FIGS. 10B-10C show calibration curves for nitrate sensor (FIG. 10B) and temporal response of pH sensor in different pH levels of water over 5 minutes at each concentration (FIG. 10C). Calibration curves for bending cycles showing minimum variation between the sensor responses (FIG. 10D). SEM images were taken for the nitrate sensor surface after 100 cycles (FIG. 10E) and 500 cycles (FIG. 10F), and 1000 cycles (FIG. 10G). At 1000 cycles there was bubble formation on nitrate sensor surface. SEM images were taken for the pH sensor surface 100 cycles (FIG. 10H), 500 cycles (FIG. 10I), and 1000 cycles (FIG. 10H). There was an appearance of a crack on the pH sensor surface at 1000 cycles.

### Example 7—Multi-Parametric Water Sensor with Nitrate Sensor: Comparison of Calibration Plots of the Nitrate Sensor in DIW and USWS

A comparison of calibration plots of the nitrate sensor in DIW and USWS was evaluated as shown in FIG. 11A. The time stability test was done in USDA water sample containing 100 ppm of nitrate for 7 days. There were slight variations observed in the voltage level, which can be attributed to the ISM layer that impeded the nitrate ion from passing through due to the gradual formation of the water layer in between the transducing layer and the ISM layer (FIG. 11B.

### Example 8—Multi-Parametric Water Sensor with Nitrate and Ammonium Sensor: Fabrication of Nitrate Sensor

All chemicals and analytical-grade reagents utilized in the experiments were employed without further purification. The deionized (DI) water utilized in the solutions exhibits a resistivity of 18.2 MΩ·cm. All chemicals utilized in this study are of analytical grade and were employed without additional purification.

The front surface of a PET substrate was configured to accommodate a nitrate sensor and an ammonium sensor positioned side by side. The reverse side of the PET substrate is configured to accommodate a temperature sensor and a pH sensor. The nitrate and ammonium sensors were prepared in a three-electrode configuration by screen-printing the electrodes on the PET substrate utilizing screen-printable inks.

This process commenced with the design of the three-electrode pattern using AutoCAD Fusion 360, followed by the transfer of this pattern onto a vinyl plotter paper with the assistance of a programmable vinyl cutter (P28 Prism Cut, USA). The vinyl pattern, which served as a mask, was affixed to a 125 μm-thick PET sheet (FIG. 12A step i). Subsequently, carbon ink was screen-printed to create the working electrode (WE) and the counter electrode (CE), while Ag ink was initially screen-printed to fabricate the reference electrode (RE). Thereafter, the upper segment of the RE was screen-printed utilizing Ag/AgCl ink (FIG. 12A step ii). The electrodes underwent annealing at a temperature of 120° C. for a duration of 15 minutes following the screen-printing process. A solution of 0.1 M CuSO₄·5H₂O (pH 2) was prepared using 0.1 M H₂SO₄, facilitating the copper nanocluster's electrodeposition. A cyclic voltammogram (CV), employing ten cycles, was applied to the working area of the WE for the electrodeposition, with a potential range spanning from −1.0 to 0 V at a scan rate of 0.1 V/s (FIG. 12A step iii). For electrodeposition, a commercial platinum wire served as the counter electrode (CE) instead of the sensor's carbon CE. Platinum used for counter electrodes in the electrodeposition technique provides excellent conductivity, chemical inertness, electrochemical stability, and catalytic properties. To prevent the leaching of chloride ions from the reference electrode (RE), a polymeric coating was created using 1.58 g of PVB, 1 g of NaCl, and 20 mL of methanol. This mixture was subjected to horn sonication at intervals, lasting 30 minutes with 5-minute breaks in an ice bath. Following this, 2 μL of the membrane was drop-cast onto the RE and allowed to air dry overnight (FIG. 12A step iv).

### Example 9—Multi-Parametric Water Sensor with Nitrate and Ammonium Sensor: Fabrication of an Ammonium Sensor

The ammonium sensor was fabricated following the same process as detailed in Example 8, above, until the electrodeposition of copper nanoclusters on the working electrode (WE) (FIG. 12B step i to FIG. 12B step iii). To enhance selectivity, conductivity, and overall performance, 5 μL of Nafion solution was applied atop the copper nanoclusters (FIG. 12B step iv). Nafion is notable for its high proton conductivity, making it ideal for sensors that require proton exchange. It aids in effective proton transfer between the electrode and the analyte, thus boosting the sensor's responsiveness and sensitivity. Given that polyaniline (PANI) is highly responsive to ammonium due to its distinctive conductivity and capacity for protonation-deprotonation reactions, the solution was prepared using 0.274 mL of aniline mixed with 5 mL of IM H₂SO₄ in 29.726 mL of distilled water. The mixture was magnetically stirred for 24 hours before use. The electrodeposition involved applying 40 CV cycles to the Nafion-coated WE via a potentiostat. This was conducted within a potential range of −0.2 to 0.7 V at a scan rate of 0.1 V/s (FIG. 12B step v). A dark greenish polyaniline layer formed on the Nafion-coated WE. The sensor was then rinsed with deionized water and dried at room temperature. Subsequently, a polymeric membrane was deposited onto the reference electrode (RE) as detailed in Example 8 (FIG. 12B step vi). Once the sensor was prepared, it was stored in a dark environment, since PANI is sensitive to light exposure and prone to photo-oxidation, particularly under ultraviolet (UV) or visible light. Extended light exposure can deteriorate PANI's conductive properties by altering its chemical structure, diminishing its sensitivity and effectiveness as a sensor.

### Example 10—Multi-Parametric Water Sensor with Nitrate and Ammonium Sensor: Fabrication of a Temperature Sensor

Following the preparation of the nitrate and ammonium sensors on the first side or surface of the PET surface, the temperature (T) and pH sensors were developed on the second side or surface of the PET substrate (FIG. 12C step i, FIG. 12D step i). The electrode's connection channel was fabricated using screen-printed Ag ink and annealed at 120° C. for 15 minutes (FIG. 12C step ii). A vinyl cutter patterned the upper arc to link two channels. To prepare the GO solution, GO powder was dispersed in DI water (1 mg/1 mL) and stirred magnetically at 500 rpm for 20 minutes. Next, the solution was sonicated for 30 minutes in 5-minute intervals, with an amplitude of 50. The semiconductive PEDOT:PSS was then mixed with the GO solution at a 1:1 ratio and stirred magnetically at 1000 rpm for 30 minutes. Subsequently, 15 μL of this mixture was drop-casted and oven-dried at 65° C. for 15 minutes (FIG. 12C step iii). This drop-casting and drying procedure was repeated. Since PEDOT:PSS and graphene oxide are sensitive to environmental factors like moisture, oxygen, and UV light, encapsulation is necessary to shield these layers from degradation due to humidity, oxygen, or other contaminants. A thin layer of PDMS was brush-coated and cured at 85° C. for 2 hours (FIG. 12C step iv). Lastly, the upper part of the device was protected with a 50 μm Kapton tape to further safeguard the sensor from environmental effects and humidity (FIG. 12C step v).

### Example 11—Multi-Parametric Water Sensor with Nitrate and Ammonium Sensor: Fabrication of a pH Sensor

Two electrodes (WE and RE) were screen-printed using Ag ink on the back of the PET substrate (FIG. 12D step i) to develop the pH sensor (FIG. 12D step ii). The working areas of both WE and RE were screen-printed again, using C and Ag/AgCl ink, respectively (FIG. 12D step iii). PANI is particularly responsive to pH alterations, making it ideal for pH sensing due to its distinct protonation-deprotonation process and the resultant changes in its electrical characteristics. Spray deposition of SWCNTs was employed to enhance the surface area of the printed Ag/AgCl RE (FIG. 12D step iv). This process adhered to a protocol formulated by Inam et al. (A. K. M. S. Inam et al., “Flexible Screen-Printed Amperometric Sensors Functionalized With Spray-Coated Carbon Nanotubes and Electrodeposited Cu Nanoclusters for Nitrate Detection,” IEEE Sens J, vol. 23, no. 20, pp. 23966-23974, October 2023) 100 layers of SWCNTs were spray deposited using a high-precision spray coater (Nordson E2 EFD, UK). Meanwhile, the WE's working area was immersed in a pre-prepared PANI solution and subjected to 40 CV cycles to electrodeposit PANI (FIG. 12D step v). Subsequently, 20 L of the polymeric membrane was drop-casted onto the RE to preserve chloride ions (FIG. 12D step vi).

### Example 12—Multi-Parametric Water Sensor with Nitrate and Ammonium Sensor: Enclosure

After fabricating the nitrate and ammonium sensors on the PET sheet's front surface and forming the pH and temperature sensors on its reverse surface, the entire PET substrate is housed inside a custom-made, waterproof 3D-printed box that includes a sensor holder. This sensor holder box was developed using a 3D printer (Qidi Tech X-Pro, USA). Another circuit enclosure box containing readout circuitry and a data communication module is connected to the sensor holder box by wires (FIG. 13A). The sensor holder box is designed to allow water entry through openings in the top cover while the data logger remains securely enclosed to protect its circuitry from water exposure. To mitigate the effects of turbulence in the water, the 3D-printed enclosure features a buffer zone or section on the top cover, isolating the sensors from direct water flow and improving measurement accuracy. This buffer zone has only two millimeter-sized holes to allow water to reach the sensor surface without turbulent water directly striking the sensors. The system-level flow diagram illustrates the processes of data acquisition, analysis, and communication between the circuit and the server, enabling multiplexed sensing (FIG. 13B).

### Example 13—Multi-Parametric Water Sensor with Nitrate and Ammonium Sensor: Cyclic Voltammetry (CV) Using Nitrate and Ammonium Sensors

A potentiostat circuit based on a three-amplifier configuration was designed to perform cyclic voltammetry (CV) on the developed nitrate and ammonium sensors. The control signal for the CV is generated by the STM32L431 microcontroller from STMicroelectronics, utilizing its 12-bit internal Digital-to-Analog Converter (DAC) and hardware timer to ensure precise voltage control and timing. The DAC output is passed through a fourth order Sallen-Key low-pass filter (LPF), which effectively suppresses high-frequency noise while minimizing phase distortion. The control amplifier (CA) drives the counter electrode to maintain a stable potential difference between the working and reference electrodes. The voltage at the RE is buffered using a high-impedance buffer to prevent loading effects and ensure accurate feedback to the control amplifier. High input impedance in the buffer may avoid or reduce drift in the RE potential. The current flowing through the WE is converted to a voltage using a variable-gain transimpedance amplifier (TIA), facilitating accurate current measurement by the microcontroller. The input bias current of the TIA must be much lower than the WE current to avoid measurement errors. The OPA4197 quad-operational amplifier from Texas Instruments was selected for the control amplifier, RE buffer, and TIA, as it offers several advantageous characteristics, including low input bias current, high input impedance, low input offset voltage, low noise, high slew rate, and excellent common-mode rejection. An LPF filters the RE voltage and TIA output and then the filtered voltages are digitized by the microcontroller's built-in 12-bit Analog-to-Digital Converter (ADC), enabling processing and data transmission. The same potentiostat circuit is used to conduct cyclic voltammetry on both nitrate and ammonium sensors, utilizing a low-resistance, bidirectional analog switch, the TMUX1574 from Texas Instruments. The microcontroller unit (MCU) firmware sets the required TIA gain and facilitates a seamless transition between sensors and ensures that the CV potential range is appropriately set for accurate measurements.

Two distinct analog front ends (AFEs) were designed to interface with the developed pH and temperature sensors, addressing their specific measurement requirements. Signal conditioning may be conducted for the pH sensor, which in some instances is characterized by its high output impedance. Its AFE consists of a low bias current and a high input impedance buffer circuit, which prevents loading effects and measurement errors. This is followed by a level-shifting amplifier (AMP), implemented using the OPA4197 operational amplifier and an RC low pass filter (LPF) stage. The AFE for the resistive temperature sensor utilizes an op-amp-based precision constant current source (CCS) to measure the sensor's resistance indirectly. A controlled current is passed through the sensor, and the resulting voltage drop across it is measured and amplified by an instrumentation amplifier (IN-AMP), INA333 from TI, level-shifted and filtered for signal conditioning. Throughout the circuit, the REF2030 voltage reference provides an accurate 3V reference to the ADC and DAC and a 1.5V reference to level-shift the DC voltages in different stages. Both AFE outputs are digitized by the internal 12-bit ADC of the same microcontroller, providing seamless integration with the data transmission circuitry.

Once the STM32L431 microcontroller acquires the data, it is transmitted to an ESP32 over a UART interface. The ESP32 acts as a wireless bridge between the STM32L431 and a remote server, leveraging its built-in Wi-Fi functionality for seamless communication. Data from the STM32L431 is formatted, packetized, and transmitted by the ESP32 using the lightweight MQTT protocol to an MQTT broker. The ThingSpeak IoT platform was utilized for testing and prototyping due to its integration with MATLAB analytics, providing powerful tools for data storage, real-time analysis, and visualization directly through a web browser.

### Example 14: Multi-Parametric Water Sensor with Nitrate and Ammonium Sensor: Morphological and Compositional Characterization of Sensors

Scanning electron microscopy (SEM) and energy dispersive spectroscopy (EDS) (JEOL, USA) was used to assess the surface morphology of the Cu nanocluster, as well as the deposition of Ag, C, and SWCNTs. Fourier Transform Infrared (FT-IR) spectroscopy was conducted to evaluate the chemical bond formations in the developed coatings, as shown in FIGS. 25A-25D.

Initially, the SEM image (FIG. 26A) of the carbon (C) electrode, primarily screen-printed with C ink for nitrate and ammonium measurements, exhibits a typical distribution of C flakes across the electrode. Subsequently, the SEM image (FIG. 26B) captured Cu electrodeposition on the C surface, displaying a uniform distribution of Cu nanoclusters, each cluster measuring around 0.5 μm in diameter. FIG. 26C shows the polyaniline (PANI) layer that appears to have a granular texture, indicating a rough, uneven surface morphology. This texture suggests a high surface area, which benefits electrochemical applications due to increased active sites. The PANI layer seems well-adhered to the underlying Nafion-coated Cu electrodeposited electrode, showing a consistent coating across the surface with no significant cracks or delamination.

Additionally, FIG. 27 shows the EDS analysis of PANI, indicating a normalized mass distribution of 50.49% C, 13.50% N, and 12.71% O. This confirms the presence of PANI on the ammonium sensor. The EDS system recorded spectra with a 30-second acquisition time and a 15 kV acceleration voltage. FT-IR bands of PANI in FIG. 25A display significant characteristic peaks at 1635, 1506, and 1203 cm⁻¹, which align with the standard PANI documented in the literature (B. Sydulu Singu, P. Srinivasan, and S. Pabba, “Benzoyl Peroxide Oxidation Route to Nano Form Polyaniline Salt Containing Dual Dopants for Pseudocapacitor,” J Electrochem Soc, vol. 159, no. 1, pp. A6-A13, 2011, doi: 10.1149/2.036201jes). Along with these peaks, PANI salt exhibits prominent bands at 3461 and 2962 cm⁻¹, corresponding to the symmetrical and asymmetrical stretching of the alkyl substituent from dodecyl hydrogen sulfate used during polymerization (W. Yin and E. Ruckenstein, “Water-soluble self-doped conducting polyaniline copolymer,” Macromolecules, vol. 33, no. 4, pp. 1129-1131, 2000, doi: 10.1021/ma991626g). The peak at 1635 cm⁻¹ signifies the presence of the aromatic —SO₃H group, while the peaks at 1056 and 502 cm⁻¹ arise from the stretching of the dopant's sulfonate (S═O) group (W. Yin and E. Ruckenstein).

On the other hand, RE of each sensor was initially screen-printed using Ag ink, while the upper half was later printed with Ag/AgCl ink, as illustrated through the SEM images in FIG. 26D and FIG. 26E. The images demonstrate a uniform distribution of Ag and Ag/AgCl nanoflakes across the electrode, with a maximum flake measuring approximately 10 μm. The SEM image of the polymeric membrane atop the Ag/AgCl electrode (FIG. 26F) reveals a porous structure marked by various well-defined holes of different sizes, likely resulting from methanol evaporation during casting. Surrounding these pores, the polymer matrix appears dense, indicating that polyvinyl butyral (PVB) has created a continuous phase that encloses NaCl particles. This porous design improves ion diffusion to the underlying reference electrode, thus enhancing electrochemical stability. The polymeric membrane made of PVB, NaCl, and methanol exhibited band patterns in FT-IR similar to those reported in the literature [36]. The O—H alcohol-stretching band is detected at 3424 cm⁻¹, confirming the presence of methanol (FIG. 26B). Furthermore, the C—H aliphatic chain at 2870 cm⁻¹, the CH3 aliphatic deformation at 1239 cm⁻¹, the secondary alcohol stretching (C—O) at 1130 cm⁻¹, and the stretching of the monosubstituted alkene (vinyl bond) at 992 cm⁻¹ collectively indicate the presence of PVB (G. E. Mcmanis and L. E. Gast, “IR Spectra of Long Chain Vinyl Derivatives,” Journal of the American Oil Chemists', vol. 48, no. 11, pp. 668-673, 1971).

Next, the temperature (T) sensor was characterized. The SEM image in FIG. 28A shows a rough, flake-like texture with irregular, layered surfaces of GO. The particles are clustered together, forming a porous structure with small gaps and voids between the layers. These characteristics suggest a high surface area and reactivity. The SEM image of PEDOT:PSS in FIG. 28B alone depicts a fibrous and wrinkled morphology with small spherical particles scattered throughout. This fibrous configuration suggests a large surface area and some flexibility, typical traits of PEDOT films. The tiny surface particles may represent leftover PSS areas or aggregation during film creation. Conversely, the GO/PEDOT:PSS nanocomposite in FIG. 28C showcases a rough, layered structure featuring clusters of GO flakes integrated within a matrix. These GO sheets appear distributed throughout the PEDOT, forming a textured and interconnected network. This design could improve electrical conductivity and mechanical strength, making the composite ideal for T sensor applications. In the FTIR spectra of PEDOT-PSS (FIG. 25C), the peaks observed at 1520 cm-1 correspond to C═C stretching within the thiophene ring of PEDOT-PSS (B. Groenendaal, F. Jonas, D. Freitag, H. Pielartzik, and J. R. Reynolds, “Poly(3,4-ethylenedioxythiophene) and Its Derivatives: Past, Present, and Future**”). The bands at 857 and 942 cm-1 are attributed to the C—S bond stretching in the quinoidal structure of the thiophene ring (C. Kvarnstroè, H. Neugebauer, S. Blomquist, H. J. Ahonen, J. Kankare, and A. Ivaska, “In situ spectroelectrochemical characterization of poly(3,4-ethylenedioxythiophene).”). Peaks at 1265 cm-1 indicate the presence of sulfonic groups (—SO3) in the PSS molecule (P. C. Mahakul et al., “Preparation and characterization of PEDOT:PSS/reduced graphene oxide-carbon nanotubes hybrid composites for transparent electrode applications,” J Mater Sci, vol. 52, no. 10, pp. 5696-5707 May 2017, doi: 10.1007/s10853-017-0806-2.). The FTIR spectrum of GO also displays absorption bands at 1160 cm-1, which relate to epoxy carbonyl groups (Y. Wu et al., “Efficient and large-scale synthesis of few-layered graphene using an arc-discharge method and conductivity studies of the resulting films,” Nano Res, vol. 3, no. 9, pp. 661-669, 2010, doi: 10.1007/s12274-010-0027-3).

Lastly, the SEM image (FIG. 28D) of SWCNTs spray-coated on top of RE to prepare the pH sensor shows thin, thread-like structures forming a dense, entangled network. The SWCNTs are uniformly distributed, creating a fibrous mesh that suggests a high surface area and strong interconnections. Additionally, EDS analysis of electrode surfaces coated with SWCNTs (FIG. 29) reveals a mass distribution of 42.68% C, 10.79% O₂, and 23.10% Au, further confirming the presence of SWCNTs on the Au-coated electrode. Besides, PANI electrodeposited on top of the C electrode for the fabrication of the pH sensor is also characterized through FT-IR. The FT-IR peak (FIG. 25D) at 3176 cm⁻¹ is attributed to C—H and C—N stretching, and another peak at 1302 cm⁻¹ is also noted (U. M. Chougale, J. V. Thombare, V. J. Fulari, and A. B. Kadam, “Synthesis of polyaniline nanofibres by SILAR method for supercapacitor application,” in 2013 International Conference on Energy Efficient Technologies for Sustainability, ICEETS 2013, 2013, pp. 1078-1083. doi: 10.1109/ICEETS.2013.6533537). Additionally, the peak at 1581 cm⁻¹ corresponds to the benzenoid ring. A peak identified at 1493 cm⁻¹ arises from the C═C vibration in the aromatic ring, indicating polymer chain formation, while the peak at 1302 cm⁻¹ is linked to the aromatic C—N stretching (S. R. Moraes, D. Huerta-Vilca, and A. J. Motheo, “Characteristics of polyaniline synthesized in phosphate buffer solution,” Eur Polym J, vol. 40, no. 9, pp. 2033-241 Sep. 2004, doi: 10.1016/j.eurpolymj.2004.05.016). Peaks between 1139 cm-1 and 1236 cm⁻¹ are associated with the protonation of PANI and represent characteristic bands (F. Cases, F. Huerta, P. Garces, E. Morallon, and J. L. Vazquez, “Voltammetric and in situ FTIRS study of the electrochemical oxidation of aniline from aqueous solutions buffered at pH 5,” vol. 2001, no. 501, December 2000, doi: https://doi.org/10.1016/S0022-0728(00)00526-X).

### Example 15: Multi-Parametric Water Sensor with Nitrate and Ammonium Sensor: Electrochemical Characterization of Nitrate Sensor

The cyclic voltammogram was recorded during each step of fabricating the nitrate sensor and exhibited distinct oxidation and reduction peaks when Cu nanoclusters were deposited on the working electrode (WE). In contrast, the WE with only carbon (C) showed no peaks (FIG. 30A). The examined potential range of the CV spanned from −0.1 to −1.4 V at a scan rate of 0.05 V/s, with a solution comprising 50 mM NaNO₃ in DI water. This confirms the catalytic reaction of Cu in the nitrate solution. Additionally, the CV revealed three distinct cathodic reduction peaks. The peaks observed at −0.4 V and −0.6 V correspond to the reduction of Cu(I) and Cu(II), respectively, as detailed in Eq. 1 and 2. The reduction of NO₃⁻ to NO₂⁻ occurs at negative potentials, illustrated in Eq. 3. This suggests that while Cu can form Cu₂O and CuO, it does not interfere with the nitrate reduction mechanism.

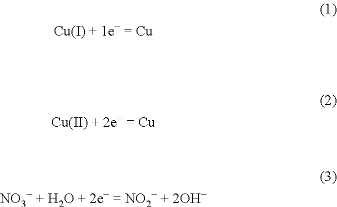

The sensor performance was assessed based on the number of CV cycles. From our previous experiment, the number of CV cycles were evaluated. The results of the test show that 10 CV cycles of Cu electrodeposition provided the excellent electroactive surface area and showed the highest sensitivity towards nitrate reduction. Exceeding 10 CV cycles can lead to excessive Cu deposition, decreasing porosity and overall surface area. Conversely, fewer than 10 cycles result in non-homogeneous Cu electrodeposition.

### Example 16: Multi-Parametric Water Sensor with Nitrate and Ammonium Sensor: Electrochemical Characterization of Ammonium Sensor

To characterize the ammonium sensor, cyclic voltammetry (CV) was performed at varying ammonium concentrations dissolved in deionized (DI) water. The potential range spanned from −1 to 0.6 V, revealing a distinct anodic peak at 0 V and a cathodic peak at −0.45 V (FIG. 30B). According to a study by Zhybak et al. (T. Zhybak et al., “Direct detection of ammonium ion by means of oxygen electrocatalysis at a copper-polyaniline composite on a screen-printed electrode,” Microchimica Acta, vol. 183, no. 6, pp. 1981-1987 Jun. 2016, doi: 10.1007/s00604-016-1834-3), which employed amperometry in deoxygenated buffer, only minor increases in anodic and cathodic currents were noted upon the addition of ammonium ions. This indicates that oxygen influences the observed cathodic currents via oxygen reduction reaction (ORR) electrocatalysis, revealing a potential limitation of this method due to its reliance on oxygen levels. When ammonium ions are present, a complex forms between Cu(I) and ammonia that is subsequently oxidized to Cu(II) with the aid of dissolved oxygen, before being electrochemically reduced back to Cu(I). The following equations represent this entire process.

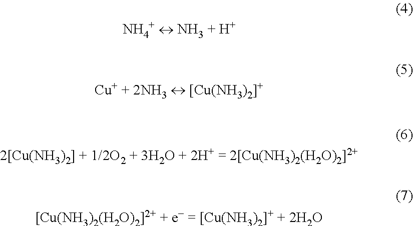

The preparation of ammonium sensors began with varying the PANI solution for electrodeposition, which involved two main steps. First, the components of the solution were mixed and magnetically stirred at 700 rpm for various durations (4, 12, and 24 hours) to assess the effect of time. Next, sensors were fabricated using these three variations and tested against five ammonium concentrations (0.1, 0.5, 1, 5, and 10 ppm) in CV experiments. As shown in FIG. 14A, stirring for four hours resulted in the highest sensitivity compared to the other durations. PANI synthesis usually occurs through chemical polymerization, such as oxidative polymerization using a substance like ammonium persulfate. The concentration of monomers and oxidants can effect the completion time as the consumption of monomers leads to chain propagation. The process may reach completion within several hours.

Over-stirring for 12 or 24 hours can lead to diminishing returns since polymerization slows down significantly as reactants become scarce. Prolonging the stirring time beyond what is necessary may result only in small increases in molecular weight or polymer uniformity, making four hours adequate for many applications. Moreover, extended stirring might induce degradation or over-oxidation of PANI, negatively impacting the polymer's conductivity and color. CV cycle was also evaluated for electrodepositing PANI onto the nafion/Cu/C electrode. CV cycle may influence the thickness, morphology, and electrochemical characteristics of the deposited film. In this study, the working area of the WEs was electrodeposited using four different CV cycles (10, 20, 40, and 60) and evaluated at three ammonium concentrations (1, 10, and 50 ppm) through CV. The sensors subjected to 40 CV cycles of PANI electrodeposition displayed the highest sensitivity (FIG. 14B) compared to the others. Fewer cycles (10 or 20) might result in a PANI layer that is too thin, restricting charge transport, while excessive cycles (like 60) could create a layer that is overly thick or dense, resulting in increased resistance. At 40 cycles, an optimal balance may occur that promotes rapid charge transport and electron transfer, thereby enhancing sensitivity.

### Example 17: Multi-Parametric Water Sensor with Nitrate and Ammonium Sensor: Performance of Nitrate Sensors

A range of nitrate concentrations (1, 10, 25, 50, and 100 ppm) in deionized (DI) water were prepared to assess the nitrate sensor's performance. Each concentration was evaluated using three sensors through cyclic voltammetry (CV). The reduction peak emerged at −1.2 V, confirming the conversion of nitrate to nitrite (FIG. 15A). The average reduction peak current was calculated for each concentration and plotted it against the nitrate levels, resulting in a linear fit that indicated a high sensitivity of 0.7128 μA/ppm for the sensor (FIG. 15B). The R² value of 0.98 demonstrates a strong linear correlation, suggesting that the sensor is both sensitive and responsive to changes in nitrate concentrations. The calibration curve illustrates a broad linear range for the sensor, spanning from 1 to 100 ppm, while maintaining good repeatability and reproducibility, as shown by the standard deviation error bars ranging from 0.18 to 6.48 μA. Additionally, all nitrate concentrations for the experiments were prepared solely with DI water, excluding any electrolytes (such as 0.1 M KCl) or acidic solutions (like Na₂SO₄ at pH 2.0. The detection limit (LOD) was calculated using the following equation, yielding a result of 0.84 ppm.

$$LOD = ( 3.3 * STDEV * {I}_{0} ) / m$$

Where I₀ is the generated peak current at 0 mM nitrate, and m is the slope of the linear response curve.

The stability of the nitrate sensor plays a vital role in identifying potential measurement drifts due to aging effects. In this experiment, three sensors were prepared from the same batch were immersed in a 50 ppm nitrate solution for a week, during which CV analysis was performed daily to determine the reduction peak current. The results presented in FIG. 15C show that the reduction in peak current from the nitrate sensor remained nearly constant during the first five days, despite a gradual day-by-day decrease being observed. The coefficients of variance (4.10% and 7.8%) for days 6 and 7 were significantly higher than those of the first five days, demonstrating the gradual degradation of the nitrate sensor. Additionally, the error bars from the standard deviation showed a similar trend. Copper is prone to corrosion in aqueous environments, particularly if dissolved oxygen, chloride ions (Cl⁻), or other aggressive species are present. Cu can oxidize to form copper oxide (CuO) or copper hydroxide (Cu(OH)₂), which may reduce the electrochemical activity and degrade the sensor's performance. In certain conditions, copper can dissolve into the water, thinning the electrodeposited layer. This reduces the active surface area available for nitrate reduction and leads to a loss in sensitivity over time.

Selectivity is crucial for assessing the reliability of nitrate sensors, as various ions interfere with nitrate detection. Five common ions (NO₂⁻, Cl⁻, PO₄³⁻, NH₄⁺, and SO₄²⁻) were tested to assess the proposed sensor's selectivity (FIG. 15D). Initially, each ion at the same concentration (50 ppm) was tested separately with a nitrate sensor. The results showed that none of the ions produced a reduction peak current when tested individually, mirroring the response of the blank concentration (DI water only). Subsequently, the individual interferents were combined with the same nitrate concentration (50 ppm) and re-evaluated using CV. Additionally, a mixture containing all the interfering ions and nitrate was prepared for evaluation. All the interferents, including the mixed ions, exhibited similar reduction peak currents of nitrate during testing, indicating minimal to no interference from common interfering ions. However, the solution containing the same concentration of NH₄⁺ and nitrate showed a lower reduction peak current because both ammonium and nitrate are nitrogen-based species, and some of their electrochemical pathways can overlap or compete. This can reduce the sensor's ability to precisely detect nitrate when ammonium is present.

### Example 18: Multi-Parametric Water Sensor with Nitrate and Ammonium Sensor: Performance of Ammonium Sensors

To assess the performance of ammonium sensors, solutions with NH₄⁺ concentrations ranging from 0.05 ppm to 10 ppm were created. Throughout the cyclic voltammetry (CV) process, the ammonium sensors displayed oxidation and reduction peaks at each concentration due to the formation of [Cu(NH₃)₂]⁺ in the presence of copper (FIG. 16A). The oxidation peaks recorded for this study were plotted against varying ammonium concentrations. Three sensors were examined for each concentration by conducting CV under experimental conditions, and the average oxidation peak current at 0 V was documented. The calibration curve (FIG. 16B) demonstrates a linear fit across a wide range of NH₄⁺ concentrations, achieving a coefficient of determination (R²) of 0.99. This reflects excellent repeatability and reproducibility, with a standard deviation between 0.025 to 0.66 μA. As expected, NH₄⁺ exhibited high sensitivity at 1.1671 μA/ppm and a low detection limit (calculated LOD 0.72 ppm). The electrodeposited polyaniline (PANI) on a nafion-coated copper (Cu) working electrode provides advantages for ammonium (NH₄⁺) detection. The cation-selective properties of nafion effectively concentrate NH₄⁺ ions at the interface, while PANI serves as a conductive matrix that efficiently transduces the electrochemical signal. Additionally, copper enhances the oxidation process at the electrode interface, further improving sensitivity in detecting ammonium. The PANI layer also stabilizes the copper electrode, reducing corrosion and offering a dependable platform for ammonium sensing.

The stability of the sensors was assessed to evaluate their shelf life by measuring the NH₄⁺ oxidation peak current daily over a week. In total, 21 sensors were prepared from the same batch and stored at room temperature (approximately 22° C.) in the dark. Each day, three sensors were tested in a pre-prepared 1 ppm NH₄⁺ solution using cyclic voltammetry (CV) to measure the oxidation peak currents and kept overnight in the same solution. The plotted average results, including error bars reflecting standard deviation values, are shown in FIG. 16C. While the reduction peak currents did not exhibit a sudden change, a progressive decline was observed following the third day. The slow decline in sensor performance over time may result from various factors. PANI is known to have limited stability in aqueous environments, especially under oxidative conditions. Continuous CV can accelerate degradation due to redox cycling, where repeated oxidation and reduction can cause morphological and structural changes in the PANI layer. Dissolved oxygen, trace ions, or impurities in the water can also contribute to degradation. Oxygen can promote oxidative degradation, while ions like chloride can corrode the electrode surface, affecting PANI's stability and sensitivity. A possible solution to enhance the stability of PANI in an aqueous solution is to implement a mediator layer or a protective coating.

Because this sensor aims to measure NH₄⁺ levels in the USDA water reservoir intended for rearing catfish, it is crucial to identify the common interferents that may arise from fish feed and waste, including Cl⁻, NO₃⁻, NO₂⁻, SO₄²⁻, and PO₄³⁻. Therefore, 5 ppm of the concentrations mentioned above were prepared with and without NH₄⁺ at the same concentration and subsequently tested for oxidation peak current using CV. The results presented in FIG. 16D indicate that the current at 0 V for all tested ions closely matches the blank concentration, with only minor variations attributed to experimental error. Additionally, the oxidation peak currents for all interfering agents were tested at the same NH₄⁺ concentration, including the mixture of all ions that matched that of NH₄⁺. This further demonstrates that the sensor experiences minimal interference from these substances. The error bars derived from the SD values showed slightly higher ranges compared to the individual ones; however, the current variations between sensors remained below 10%.

### Example 19: Multi-Parametric Water Sensor with Nitrate and Ammonium Sensor: Performance of Temperature and pH Sensors

Following the fabrication process, 15 temperature (T) sensors were produced in a single batch and evaluated across a broad temperature range from 5° C. to 45° C. The sensors, linked through a multimeter to track resistance variations, were immersed in a USDA water sample. Resistance was measured for 5 minutes at each temperature level, and the data was recorded. As shown in FIG. 17A, the resistance range remained relatively constant at each temperature level. PEDOT is a conducting polymer that functions as a thermistor, which means its conductivity rises with temperature. As the temperature increases, the thermal energy excites more charge carriers, increasing conductivity and decreasing resistance. In this context, PSS serves as a dopant, supplying mobile ions. Higher temperatures enhance the mobility of these ions, further reducing resistance by facilitating the movement of charge carriers within the material. Collectively, these factors enable the GO and PEDOT composite sensor to act as a negative temperature coefficient (NTC) thermistor, where resistance diminishes as temperature rises, as shown in FIG. 17B. To calibrate the T sensor, average readings from three different sensors were collected at various temperatures and shown with a clear linear fit in relation to temperature, as displayed in FIG. 17B. The calibration plot, with error bars derived from standard deviation (SD) values, demonstrated high sensitivity (−2.99 kΩ/° C.) and an excellent coefficient of determination (R²=0.99). The reproducibility of three different sensors is shown in FIG. 17C.

The pH sensor consists of two electrodes: WE, where PANI was electrodeposited on top of the working area, and the Ag/AgCl RE, where SWCNTs were spray-coated, followed by the deposition of a polymeric membrane. Here, an open circuit potential (OCP) observed between the WE and RE demonstrated the pH sensor's electrochemical properties. The PANI-based WE selectively responds to H₃O⁺, along with the RE, producing potential differences in response to pH variations in the solution. PANI is a conducting polymer that reacts to pH changes via protonation and deprotonation. PANI becomes protonated in acidic conditions (low pH), increasing conductivity and electrode potential. Conversely, as the pH rises (becoming more basic), PANI deprotonates, losing protons, which leads to reduced conductivity and a lower potential. To evaluate the sensor's performance, solutions with pH levels ranging from 3 to 11 were prepared using an acid and a base in the USDA water sample. A multimeter recorded the OCP of three pH sensors over five minutes. FIG. 17D illustrates the average OCP values at each pH level, showing consistent values across all levels. The pH sensor calibration was achieved by averaging the readings of each sensor at different pH levels and plotting these averages against the pH levels. The calibration plot in FIG. 17E demonstrated a linear fit, indicating high sensitivity of the pH sensor (−0.0229 V/pH) and an outstanding R² value (0.99). The smaller error bars from the standard deviation also demonstrate the pH sensor's precision, reliability, and consistency. The OCP values of three pH sensors was plotted by averaging readings at each pH level showing the sensor's reliability (FIG. 17F).

After fabricating and calibrating the T sensor, additional sensors were produced in the same batch to assess the temperature in an USDA fish tank, denoted as T1. Water samples were collected from the T1 tank at six time points, denoted as −2 hr, −1 hr, 0 hr, 1 hr, 2 hr, and 24 hr. These represent 2 hours before NH₄⁺ dosing, 1 hour before, immediately after, and 1-, 2-, and 24-hours post-dosing, respectively. The temperature sensors were used to measure temperatures of all six water samples. The resistance readings (in kΩ) obtained from the T sensor measurements were transformed into temperature values using the calibration equation (FIGS. 17B-17C) and displayed in FIG. 18A. All water samples were stored at room temperature (23° C.), resulting in minimal or no variations in the values captured by the temperature sensors. The T sensor measurements for all six samples indicated approximately 23° C., confirming the sensor's reliability. During the day, the temperature in fish tanks or ponds rises, while it drops at night. The reaction kinetics of the nitrate and ammonium sensors are influenced by temperature and may change with fluctuations. Consequently, the integrated temperature sensor accurately assesses the current temperature.

Similar to temperature, pH is also a crucial parameter that varies in various ways and influences nitrate and ammonium measurements. In particular, the NH₄⁺ sensor, which was prepared by the electrodeposition of PANI, is highly pH sensitive due to its protonation behavior. PANI loses protons (deprotonates) as pH increases and becomes less conductive. This pH-dependent variation in conductivity can influence the sensor's response to ammonium, potentially impacting both the sensitivity and stability of the sensor. Thus, the pH sensor was integrated into the sensor platform. The pH levels of all USDA fish tanks was assessed, each containing six different samples, and compared these results with those from the Laqua sensor kit (HORIBA Group, USA). As shown in FIG. 18B, pH measurements from both sensor and the Laqua kit ranged from approximately 7 to 8.5. Notably, the pH level initially decreased over time and during NH₄Cl dosing (3 ppm), and after that, it gradually rose again. This trend was observed consistently across all six tanks, and the pH sensor readings reflected similar patterns. The nitrate and ammonium sensors are calibrated at the same pH as the target location for accurate results, which can be achieved with the pH sensor.

### Example 20: Multi-Parametric Water Sensor with Nitrate and Ammonium Sensor: Monitoring of USDA Fish Tank Water

The performance of the sensor was evaluated in aquaculture settings, which often experience rapid changes in temperature and pH. FIG. 31 shows the sensor system immersed in a catfish recirculating aquaculture system (RAS) at the USDA Agricultural Research Service's (USDA-ARS) Warmwater Aquaculture Research Unit in Stoneville, MS, USA. Water samples from this RAS system was used for all subsequent analyses. FIG. 32 shows the integrated sensor system, showing the final data transmitted to a mobile phone via WiFi. Sensors were tested in six tanks (T1, T3, T4, T6, T7 and T9), each containing 10 catfish and equipped with an automatic aeration system. 3 ppm of NH4Cl was added to each tank at specific intervals. Data were collected from our sensors at six time points: prior to dosing (2 hours and 1 hour), immediately after dosing (0 hr), and post-dosing (1 hour, 2 hours, and 24 hours). First, the nitrate and ammonium sensors were calibrated using water from the T1 tank, with adjustments made according to the tank's pH. We also measured the water temperature with our T sensor and the temperature was found consistent across all tanks. Likewise, the water pH in the six tanks were measured with our pH sensor and the pH level was found pretty similar across all tanks. Nitrate and ammonium concentrations were measured using our sensor system in conjunction with a standard measurement kit supplied by USDA-ARS for comparative analysis.

The results presented in FIG. 19A illustrate that the nitrate concentration increased hourly due to the natural biological process known as the nitrogen cycle, wherein bacteria in the tank convert fish waste and other nitrogenous compounds into nitrate. It is also evident that the nitrate levels recorded by the sensor and the measurement kit exhibited a similar trend. The average nitrate concentration measured by both systems ranges from 70 to 80 parts per million (ppm), which is standard for a fish tank; however, Tanks T3, T4, and T6 demonstrated elevated nitrate levels exceeding 100 ppm. Ammonium levels were measured in all six tanks using the sensor system and the ammonium kit provided by USDA-ARS. As shown in FIG. 19B, the kit detected the lowest ammonium values (<100 ppb) in all tanks one and two hours before and 24 hours after dosing. During dosing with 3 ppm of NH₄Cl, both the sensor and kit data indicated the same NH₄⁺ levels, which gradually began to decline after one and two hours. This reduction in the NH₄⁺ content was indicated from both the kit and the ammonium sensor. Without being held to a particular theory, it is believed that the primary reason for the drop in ammonium concentration is the nitrification process, which is performed by nitrifying bacteria present in the tank. These bacteria, particularly Nitrosomonas, convert NH₄⁺ into NO₂⁻ as part of the nitrogen cycle. Ammonium and ammonia exist in a pH-dependent equilibrium. If the tank's pH increases, more ammonium ions convert to ammonia, which can escape as gas, reducing the concentration of measurable ammonium, though this effect is more noticeable at higher pH levels.

To observe the differences between the measurements obtained from the sensor and those from the commercial kits, the nitrate and ammonium values recorded by the sensors were plotted against the corresponding values measured by the commercial kits, as illustrated in FIGS. 19C and 19D. FIG. 19C indicates that the root mean square error (RMSE) between the sensor and the commercial kit is 6.241 ppm, further proving the sensor's high accuracy. FIG. 19D shows the scatter plots of ammonium measured by kit versus ammonium measured by the sensor. The calculated RMSE of 0.4786 ppm for the ammonium sensor illustrates the excellent accuracy of the ammonium sensor. The correlation between sensor measurements and kit values can be relatively low if the temperature and pH are compensated. Furthermore, notable R² values for nitrate (0.838) and ammonium (0.847) were observed, as shown in FIGS. 19C and 19D, where the data points are closer to the linear fit.

### Example 21: Multi-Parametric Water Sensor with Nitrate and Ammonium Sensor: Evaluating the Integrated Sensor System

The integrated sensor measures the potential of pH sensors and the resistance of temperature sensors, while also conducting cyclic voltammetry (CV) for nitrate and ammonium sensors. FIG. 20 shows a comprehensive comparison between the commercial potentiostat (Basi EmStat4S) and an exemplary embodiment according to the aspects disclosed herein-(“developed”) potentiostat for cyclic voltammetry (CV) assessments. Nitrate measurements were taken at five specific concentrations (1 ppm, 10 ppm, 25 ppm, 50 ppm, and 100 ppm) using both potentiostats, within an applied potential range from −1.4V to −0.1V at a scan rate of 0.05 V/s, as illustrated in FIGS. 20A-20B. Both devices showed nitrate reduction peaks at identical voltages with comparable current responses. In FIG. 20C, reduction peak currents are plotted against nitrate concentrations to generate calibration data points, with the largest deviation for nitrate recorded at 4.2% at a concentration of 25 ppm. A comparable analysis was performed for ammonium ion detection at concentrations of 0.5 ppm, 1 ppm, 5 ppm, and 10 ppm, in a potential range from −1.0V to +0.6V and at a scan rate of 0.05 V/s, as depicted in FIGS. 20D-20E. The calibration points for ammonium, based on the oxidation peaks, are shown in FIG. 20F, indicating a maximum discrepancy of 8.8% at 1 ppm. These slight discrepancies noted between the two potentiostats may stem from differences in hardware components and current measurement algorithms. The integrated potentiostat utilized in this study is designed to measure the potential of pH sensors and the resistance of temperature sensors. Additionally, it conducts cyclic voltammetry (CV) for the analysis of nitrate and ammonium sensors. An onboard microcontroller handles the data processing. Furthermore, the integrated WiFi module allows the potentiostat to connect to an IoT server, providing remote access and monitoring of unknown nitrate and ammonia levels, in addition to real-time pH and temperature readings.

The measured data is then processed by an onboard microcontroller and transmitted to an IoT server, enabling remote access to unknown concentrations of nitrate and ammonia, as well as pH and temperature readings (FIG. 21).

### OTHER EMBODIMENTS

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims. Section headings, the materials, methods, and examples are illustrative only and not intended to be limiting.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

Other aspects, advantages, and modifications are within the scope of the following claims.

What is claimed is:

1. A multi-parametric water sensor, comprising:
a substrate;
a sensor array; and
an enclosure comprising a water impermeable material;
the sensor array comprising:
an analyte sensor comprising a working electrode (WE) and a reference electrode (RE) disposed on a first surface of the substrate;
a pH sensor comprising a working electrode (WE) and a reference electrode (RE) disposed on the first surface or a second surface of the substrate; and
a temperature sensor disposed on the first surface or a second surface of the substrate; and
wherein the enclosure comprises a sensor section and a water permeable section.

2. The multi-parametric water sensor of claim 1, wherein:
a) the analyte is an ion, a dissolved gas, or a combination thereof,
b) the analyte sensor is a potentiometric sensor,
c) the analyte sensor is an ammonia sensor,
d) the analyte sensor is a nitrate sensor, or
e) the ammonia sensor measures total ammonia, wherein the total ammonia is the sum of ammonium (NH₄⁺) and ammonia (NH₃) in solution.

3. The multi-parametric water sensor of claim 2, wherein the nitrate sensor comprises a poly(3-octyl-thiophene) (POT)-MoS₂ nanocomposite, optionally wherein the ratio of POT to MoS₂ is about 1:1 to about 1:6, about 1:2 to about 1:5, about 1:3 to about 1:5, or about 1:4 to about 1:5.

4. The multi-parametric water sensor of claim 2, wherein the nitrate sensor comprises an ion selective membrane (ISM), optionally wherein the (POT)-MoS₂ nanocomposite is immobilized in the ISM, optionally wherein the ISM is selective for nitrate ions, optionally wherein the ISM is uniform, optionally wherein the nitrate sensor is selective for nitrate over interring ions, wherein the nitrate sensor is selective for nitrate over interring ions.

5. The multi-parametric water sensor of claim 1, wherein the nitrate sensor is selective for nitrate over Cl⁻, SO₄²⁻, HCO₃⁻, PO₄³⁻, and NO₂⁻, or a combination thereof.

6. The multi-parametric water sensor of claim 1, wherein the RE comprises silver, optionally wherein the RE comprises Ag/AgCl.

7. The multi-parametric water sensor of claim 6, wherein the RE further comprises a single-walled carbon nanotube (SWCNT) layer.

8. The multi-parametric water sensor of claim 7, wherein the RE further comprises a poly (vinyl butyral) (PVB) coating.

9. The multi-parametric water sensor of claim 1, wherein the pH sensor comprises electrodeposited gold.

10. The multi-parametric water sensor of claim 1, wherein the pH sensor further comprises a thermoplastic resin membrane, optionally wherein the thermoplastic resin is a polyaniline (PANI) resin.

11. The multi-parametric water sensor of claim 1, wherein the analyte sensor RE and the pH sensor RE is the same RE electrode, and wherein the RE is in communication with both the analyte sensor and the pH sensor.

12. The multi-parametric water sensor of claim 1, wherein the temperature sensor comprises a graphene oxide-Poly(3,4-ethylenedioxythiophene):polystyrene sulfonate (GO-PEDOT:PSS) composite, optionally wherein the temperature sensor further comprises a polydimethylsiloxane (PDMS) layer, optionally wherein the temperature sensor further comprises a polyimide film.

13. The multi-parametric water sensor of claim 1, wherein the multi-parametric water sensor comprises two analyte sensors, optionally wherein the two analyte sensors are disposed on the first surface of the substrate, optionally wherein the two analyte sensors are configured to resist biofouling.

14. The multi-parametric water sensor of claim 13, wherein the pH sensor and the temperature sensor are disposed on the second surface of the substrate.

15. The multi-parametric water sensor of claim 13, wherein a first analyte sensor comprises a nitrate sensor and a second analyte sensor comprises an ammonia sensor.

16. A multi-parametric water sensor, comprising:
a substrate;
a sensor array; and
an enclosure comprising a water impermeable material;
the sensor array comprising:
a first analyte sensor comprising a working electrode (WE), a counter electrode (CE), and a reference electrode (RE) disposed on a first surface of the substrate;
a second analyte sensor comprising a working electrode (WE), a counter electrode (CE), and a reference electrode (RE) disposed on the first surface of the substrate;
a pH sensor comprising a working electrode (WE) and a reference electrode (RE) disposed on a second surface of the substrate; and
a temperature sensor disposed on the second surface of the substrate; and
wherein the enclosure comprises a sensor section and a water permeable section.

17. The multi-parametric water sensor of claim 16, wherein:
a) the first analyte sensor is a nitrate sensor,
b) wherein the nitrate sensor WE and the CE comprise carbon,
c) wherein the nitrate sensor WE further comprises copper,
d) wherein the nitrate sensor RE comprises silver, optionally wherein the nitrate sensor RE comprise Ag/AgCl,
e) the nitrate sensor CE comprises platinum,
f) the nitrate sensor RE further comprises a poly (vinyl butyral) (PVB) coating, g) the second analyte sensor is an ammonium sensor,
h) wherein the ammonium sensor WE and the CE comprise carbon, optionally wherein the ammonium sensor WE further comprises copper,
i) wherein the ammonium sensor WE further comprises a perfluorinated polymer coating comprising a polytetrafluoroethylene (PTFE),
j) wherein the ammonium sensor WE further comprises a thermoplastic resin membrane, optionally wherein the thermoplastic resin is a polyaniline (PANI) resin,
k) wherein the ammonium sensor RE comprise silver, optionally wherein the ammonium sensor RE comprise Ag/AgCl,
l) wherein the ammonium sensor RE further comprises a poly (vinyl butyral) (PVB) coating,
m) wherein the ammonium sensor CE comprises platinum,
n) wherein the temperature sensor comprises silver, or
o) wherein the pH sensor comprises a working electrode (WE) and a reference electrode (RE).

18. The multi-parametric water sensor of claim 7, wherein the pH sensor RE further comprises a single-walled carbon nanotube (SWCNT) layer, optionally wherein the pH sensor RE further comprises a poly (vinyl butyral) (PVB) coating.

19. The multi-parametric water sensor of claim 16, wherein the temperature sensor comprises a graphene oxide-Poly(3,4-ethylenedioxythiophene):polystyrene sulfonate (GO-PEDOT:PSS) composite, optionally wherein the temperature sensor further comprises a polydimethylsiloxane (PDMS) layer, optionally wherein the temperature sensor further comprises a polyimide film.

20. The multi-parametric water sensor of claim 16, wherein:
a) the enclosure further comprises a buffer zone between the sensor section and the water permeable section, or
b) the substrate has a thickness of 50 μm to 500 μm, or 75 μm to 400 μm, or 100 μm to 350 μm, or 110 μm to 250 μm, or 115 μm to 200 μm.

21. The multi-parametric water sensor claim 16, further comprising a data acquisition system (DAS), wherein the DAS comprises a processor; a communication unit; a memory unit; and a power supply unit, and wherein the data acquisition system is in communication with the sensor array, optionally:
a) wherein the memory unit is communicatively coupled to the processor, the memory unit having stored thereon computer software comprising a set of instructions that, when executed by the processor, causes the data acquisition unit to receive sensor data from the sensor array; and send, via the communication unit, the sensor data to an external device,
b) wherein the memory unit comprises a non-transitory computer-readable medium,
c) wherein the sensor data are sent, via the communication unit, to an Internet of Things (IoT) cloud server configured to interact with one or more IoT-capable devices,
d) wherein the DAS comprises a microcontroller unit,
e) wherein the enclosure further comprises a DAS section, wherein the DAS section separates the DAS from the water,
f) wherein the instructions comprise a signal calibration of at least one electrode,
g) wherein the instructions comprise a pH-based signal correction, a temperature-based signal correction, or a combination thereof,
h) wherein the analyte sensor has an analyte detection range of about 0.7 ppm or more, about 0.8 ppm or more, about 0.7 ppm to about 100 ppm, or about 0.8 to about 100 ppm,
i) wherein the nitrate sensor has an analyte detection range of about 0.8 ppm to about 100 ppm,
j) wherein the ammonium sensor has an analyte detection range of about 0.7 ppm to about 10 ppm,
k) wherein the analyte sensor has a coefficient of variance between measurements of not more than about 10%, not more than about 8%, not more than about 7%, not more than about 6%, not more than about 5%, not more than about 4%, not more than about 2%, or not more than about 1%, and/or
l) wherein the first analyte sensor, the second analyte sensor, or both has a coefficient of variance between measurements of not more than about 10%, not more than about 8%, not more than about 7%, not more than about 6%, not more than about 5%, not more than about 4%, not more than about 2%, or not more than about 1%.

22. A method of measuring one or more analytes in an aqueous environment, comprising:
placing the multi-parametric water sensor of claim 16 in the aqueous environment;
measuring a signal corresponding to one or more analytes in the aqueous environment;
measuring at least one environmental parameter comprising:
i) measuring a signal corresponding to a pH of the aqueous environment;
ii) measuring a signal corresponding to a temperature of the aqueous environment; or
iii) both i) and ii); and
obtaining as a data output from the multi-parametric water sensor an amount of the one or more analytes in the aqueous environment, wherein the amount of at least one of the analytes is based on the measured value of the at least one environmental parameter of the aqueous environment.

23. The method of claim 22, further comprising applying an electrochemical potential across one or more sensors of the sensor array to prevent or reduce biofouling on the one or more sensors, optionally wherein the environmental parameter comprises measuring a signal corresponding to a pH of the aqueous environment and measuring a signal corresponding to a temperature of the aqueous environment.

24. The method of claim 22, wherein:
a) the method comprises measuring total ammonia in the aqueous environment, optionally further comprising measuring the signal corresponding to the pH of the aqueous environment, measuring the signal corresponding to the temperature of the aqueous environment, and determining the ratio of ammonium (NH₄⁺) to ammonia (NH₃) in the aqueous environment,
b) the method further comprises measuring a signal corresponding to an amount of nitrate in the aqueous environment,
c) the method further comprises measuring a signal corresponding to an amount of nitrate in the aqueous environment,
d) each of the measuring steps comprises continuous measurement,
e) the method further comprises measuring a signal corresponding to an amount of nitrate in the aqueous environment,
f) each of the measuring steps comprises continuous measurement,
g) each of the measuring steps comprises measurement every 1 minute to 10 minutes or every 2 minutes to 3 minutes,
h) the multi-parametric water sensor processes the sensor data from the one or more sensors to produce the data output,
i) the processed data are stored in the memory unit of the multi-parametric water sensor,
j) the processed data are sent, via the communication unit, to the IoT cloud server configured to interact with the one or more IoT-capable devices,
k) one or more of the measuring steps, the data output, or both is conducted automatically by the multi-parametric water sensor without user intervention, optionally wherein each of the measuring steps and the data output is conducted automatically by the multi-parametric water sensor without user intervention after a step of activating the multi-parametric water sensor,
l) the data output is obtained from the multi-parametric water sensor,
m) the data output is obtained from the IoT capable device, and/or
n) the aqueous environment comprises an environment selected from an aquaculture, a hydroponic system, an aquaponic system, watersheds, streams, ponds, lakes, rivers, water reservoirs, fisheries, or wastewater systems.

25. A method for preparing a multi-parametric water sensor, comprising:
preparing a sensor array, comprising:
a) disposing a pH sensor working electrode (WE) pattern and an analyte WE pattern on a substrate;
disposing a reference electrode (RE) patten on the substrate between the analyte WE pattern and the pH sensor WE pattern;
disposing a temperature sensor WE pattern comprising a first pattern and second pattern on the substrate, wherein the analyte sensor WE, the RE, and the pH WE are disposed between the first pattern and the second pattern;
printing an ink comprising silver on the analyte sensor WE pattern and the temperature sensor WE pattern;
printing an ink comprising carbon on the pH sensor pattern;
printing an ink comprising Ag/AgCl on the RE pattern;
electrodepositing a gold layer onto the pH WE carbon ink layer;
depositing a GO/PEDOT:PSS connection layer connecting the first and second temperature sensor coated patterns; and
coating a PoT-MoS₂ layer onto the analyte WE silver ink layer; or
b) disposing on a first surface of a substrate a first analyte sensor working electrode (WE) pattern, a counter electrode (CE) pattern, and a reference electrode (RE) pattern;
disposing on the first surface of the substrate a second analyte sensor WE pattern, a CE pattern, and a RE pattern;
disposing on a second surface of the substrate a pH sensor WE pattern and RE pattern;
disposing on the second surface of the substrate a temperature sensor first connection channel pattern and second connection channel pattern;
printing an ink comprising carbon on the first analyte sensor WE and CE patterns, the second analyte sensor WE and CE patterns, and the pH sensor WE pattern;
printing an ink comprising silver on the first analyte sensor, the second analyte sensor, pH sensor RE patterns, and the first connection channel pattern and the second connection channel pattern of the temperature sensor;
printing an ink comprising Ag/AgCl on a portion of the first analyte sensor and a portion of the second analyte sensor RE patterns;
electrodepositing copper on the first analyte sensor and the second analyte sensor WE pattern;
electrodepositing on the second analyte sensor copper layer a perfluorinated polymer coating comprising a polytetrafluoroethylene (PTFE);
electrodepositing on the second analyte sensor copper and perfluorinated polymer coating layers a polyaniline (PANI) layer;
electrodepositing on the pH sensor WE carbon layer a polyaniline (PANI) layer; and
depositing a GO/PEDOT:PSS connection layer connecting the first connection channel pattern and the second connection channel pattern of the temperature sensor.
